(19) United States
(12) Patent Application Publication
(10) Pub. No.: US 2010/0279334 A1
(43) Pub. Date: Nov. 4, 2010

(54) CYCLODIPEPTIDE SYNTHASES (CDSS) AND THEIR USE IN THE SYNTHESIS OF LINEAR DIPEPTIDES

(75) Inventors: Ludovic Sauguet, Paris (FR); Robert Thai, Nozay (FR); Pascal Belin, Igny (FR); Alain Lecoq, Mennecy (FR); Roger Genet, Limours-En-Hurepoix (FR); Jean-Luc Pernodet, Cachan (FR); Muriel Gondry, Gif-Sur-Yvette (FR)

Correspondence Address: THE NATH LAW GROUP, 112 South West Street, Alexandria, VA, 22314, US

(73) Assignees: KYOWA HAKKO BIO CO., LTD, Tokyo (JP); CENTRE NATIONAL DE LA RECHERCHE SCIENTIFIQUE, Paris (FR); UNIVERSITE PARIS SUD 11, Orsay (FR); COMMISSARIAT A L'ENERGIE ATOMIQUE ET AUX ENERGIES ALTERNATIVES, Paris (FR)

(21) Appl. No.: 12/740,411

(22) PCT Filed: Oct. 31, 2007
(86) PCT No.: PCT/IB07/04231

§ 371 Date: Apr. 29, 2010

(51) Int. Cl. C12Q 1/02; C12P 21/06; C12N 15/63

(52) U.S. Cl. 435/29; 435/69.1; 435/320.1

(57) ABSTRACT

Use of CDSs in the synthesis of linear dipeptides, and applications thereof for the in vivo and in vitro synthesis of linear dipeptides, in particular Phe-Leu, Leu-Phe, Phe-Phe, Phe-Tyr, Tyr-Phe, Leu-Leu, Leu-Tyr, Tyr-Leu, Phe-Met, Met-Phe, Leu-Met, Met-Leu, Tyr-Met, Met-Tyr, Met-Met, Tyr-Tyr, Ile-Met, Met-Ile, Leu-Ile, Ile-Leu using the corresponding polynucleotides.

The present invention relates to the use of CDSs in the synthesis of linear dipeptides (also called hereinafter straight-chain dipeptides), and the applications thereof for the in vivo and in vitro synthesis of linear dipeptides, in particular Phe-Leu, Leu-Phe, Phe-Phe, Phe-Tyr, Tyr-Phe, Leu-Leu, Leu-Tyr, Tyr-Leu, Phe-Met, Met-Phe, Leu-Met, Met-Leu, Tyr-Met, Met-Tyr, Met-Met, Tyr-Tyr, Ile-Met, Met-Ile, Leu-Ile, Ile-Leu using the corresponding polynucleotides.

Useful properties have already been demonstrated for some linear dipeptides and their derivatives in various fields such as pharmaceuticals, health-care products, food-supplements, cosmetics and the like.

For example, the Val-Tyr and Ile-Tyr dipeptides have been shown to inhibit angiotensin-converting enzyme (ACE) activity (Maruyama et al., J. Jpn. Soc. Food Sci. Technol. 2003, 50, 310-315) and they also have an in vivo antihypertensive effect (Tokunaga et al., J. Jpn. Soc. Food Sci. Technol. 2003, 50, 457-462; Matsui et al., Clin. Exp. Pharmacol. Physiol., 2003, 4, 262-265). Many other dipeptides (e.g. Val-Trp, Val-Phe, Ile-Trp, Ala-Tyr) are also known as ACE inhibitory products (Das and Soffer, J. Biol. Chem., 1975, 250, 6762-6768; Cheung et al., J. Biol. Chem., 1980, 255, 401-407).

Kyotorphin (Tyr-Arg), a neurodipeptide first isolated in the bovine brain and later found in the brains of many other species including humans (Takagi et al., Nature, 1979, 282, 410-412; Shiomi et al., Neuropharmacology, 1981, 20, 633-638), has also been shown to be a bioactive molecule. It possesses various opioid activities, including analgesic effects (Bean and Vaught, Eur. J. Pharmacol., 1984, 105, 333-337). D-Kyotorphin (i.e. Tyr-D-Arg) or N-methylated kyotorphin (i.e. TyrΨ[CON(Me)]-Arg) analogues exhibit a stronger in vivo analgesic effect than that of natural kyotorphin, probably due to their better resistance to peptide degradation (Takagi et al., CMLS, 1982, 38, 1344-1345; Ueda et al., Peptides, 2000, 21, 717-722).

Other examples of useful dipeptides are carnosine (B-Ala-His) and homocarnosine (γ-aminobutyryl-His) that are found in several human tissues. Their physiological functions are unknown although various potential prophylactic or therapeutic applications in diabetic secondary complications (e.g. cataracts), atherosclerosis, cancer or inflammatory diseases have been reported (see Hipkiss, Int. J. Biochem. Cell Biol., 1998, 30, 863-868). Carnosine is presently used as a supplementation nutrient in human health because it is believed to delay senescence and provoke cellular rejuvenation.

Linear dipeptides are also found in some nutritional supplements, particularly those marketed as sports and fitness products but also in total parenteral nutrition (TPN) and intravenous nutrition (IVN) products. They are used as delivery forms of amino acids that are unstable and insoluble in water such as glutamine or tyrosine.

Gly-Gln and Ala-Gln are used in TPN (Jiang et al., J. Parenter. Enteral Nut., 1993, 17, 134-141) to compensate for glutamine depletion which is a feature of metabolic stress such as trauma, infection, or cancer (Zhou et al., J. Parenter. Enteral Nut., 2003, 27, 241-245).

In the same way, Ala-Tyr, Gly-Tyr and Tyr-Arg are used in IVN for providing tyrosine amino acid in an easily administrable form (Kee and Smith, Nutrition, 1996, 12, 577-577; Himmelseher et al., J. Parenter. Enteral Nut., 1996, 20, 281-286).

Finally, linear dipeptides are also used in the food industry as flavoring agents as exemplified by the aspartame molecule (Asp-Phe-OMe), which is used as a sugar substitute marketed worldwide. It is often provided as a table condiment and it is commonly used in diet food or drinks.

Known methods for producing linear dipeptides include chemical synthesis, extraction from natural producer organisms and also enzymatic methods.

Chemical methods can be used to synthesize dipeptide derivatives but they are considered to be disadvantageous with respect to cost as they often necessitate the use of protected and deprotected steps in the linear dipeptide synthesis. Moreover, they are not environment-friendly methods as they use large amounts of organic solvents and the like.

Extraction of linear dipeptides from natural prokaryote or eukaryote producers can be used but the productivity and yield is generally low because the overall content of a desired dipeptide derivative in natural products is often low and producer organisms can be difficult to manipulate. Another significant disadvantage is that all potential linear dipeptides are generally not present in a single natural (e.g. genetically unaltered) product or organism.

Enzymatic methods, i.e. methods utilizing enzymes either in vivo (e.g. in the culture of microorganisms expressing endogenous or heterologous dipeptide-synthesizing enzymes or microorganism cells isolated from the culture medium) or in vitro (e.g. purified dipeptide-synthesizing enzymes) can be used.

The following methods are already known:

A method utilizing a reverse reaction of protease (Bergmann and Fraenkel-Conrat, J. Biol. Chem., 1937, 119, 707-720); however, the method utilizing a reverse reaction of protease requires the introduction and removal of protective groups for functional groups of the amino acids used as substrates, which causes difficulties in raising the efficiency of the peptide-forming reaction and in preventing a peptidolytic reaction.

Methods utilizing thermostable aminoacyl t-RNA synthetase (Japanese Patent Application N^{o} 146539/83, Japanese Patent Application N^{o} 209991/83, Japanese Patent Application N^{o} 209992/83 and Japanese Patent Application N^{o} 106298/84); the methods utilizing thermostable aminoacyl t-RNA synthetase have problems in that the expression of this enzyme and the prevention of side reactions forming unwanted by-products other than the desired products are difficult to prevent.

A method utilizing reverse reaction of proline iminopeptidase (WO03/010307); the method utilizing proline iminopeptidase requires amidation of one of the amino acids used as substrates, which again makes such methods difficult to conduct.

Methods utilizing non-ribosomal peptide synthetase (hereinafter referred to as NRPS) (Doekel and Marahiel, Chem. Biol., 2000, 7, 373-384; Dieckmann et al., FEBS Lett., 2001, 498, 42-45; U.S. Pat. No. 5,795,738 and U.S. Pat. No. 5,652,116). The methods utilizing NRPS are inefficient in that the supply of coenzyme 4′-phosphopantetheine is necessary.

There also exists a group of peptide synthetases that have lower enzyme molecular weights than that of NRPS and do not require coenzyme 4′-phosphopantetheine; for example, gamma-glutamylcysteine synthetase, glutathione synthetase, D-alanyl-D-alanine (D-Ala-D-Ala) ligase, and poly-gamma-glutamate synthetase. Most of these enzymes utilize D-amino acids as substrates or catalyze peptide bond formation at the gamma-carboxyl group. As a result of this, they cannot be used for the synthesis of dipeptides by peptide bond formation at the alpha-carboxyl group of L-amino acid.

An example of an enzyme capable of dipeptide synthesis by forming a peptide bond at the alpha-carboxyl group of L-amino acid is bacilysin synthetase (bacilysin is a dipeptide antibiotic derived from a microorganism belonging to the genus Bacillus). Bacilysin synthetase is known to have the activity to synthesize bacilysin [L-alanyl-L-anticapsin (L-Ala-L-anticapsin)] and L-alanyl-L-alanine (L-Ala-L-Ala), but there is no information about its ability to synthesize other dipeptides (Sakajoh et al., J. Ind. Microbiol. Biotechnol., 1987, 2, 201-208; Yazgan et al., Enzyme Microbial Technol., 2001, 29, 400-406).

As for the bacilysin biosynthetase genes in Bacillus subtilis 168 whose entire genome has been sequenced (Kunst et al., Nature, 1997, 390, 249-256), it is known that the productivity of bacilysin is increased by amplification of bacilysin operons containing ORFs ywfA-F (WO00/03009).

Recently, it has been demonstrated that the ywfE ORF encodes a L-amino acid ligase responsible for the synthesis of alpha-dipeptides from L-amino acids substrates. The enzyme was shown to have a broad substrate specificity leading to the formation of a wide variety of alpha-dipeptides (Tabata et al., J. Bacteriol., 2005, 187, 5195-5202; U.S. Patent Application No 20050287626).

The Inventors have previously reported that AlbC (albC gene product), which has no similarities with NRPS, was responsible for the formation of cyclo(L-Phe-L-Leu) and cyclo(L-Phe-L-Phe) during the biosynthesis of the anti-bacterial substance albonoursin (cyclo(deltaPhe-deltaLeu)) in Streptomyces noursei ATCC 11455. The expression of AlbC from S. noursei in heterologous strain S. lividans TK21 or Escherichia coli led to the production of cyclo(L-Phe-L-Leu) and cyclo(L-Phe-L-Phe) that were secreted in the culture medium (Lautru et al., Chem. Biol., 2002, 9, 1355-1364; French Patent 2841260 and WO2004/000879).

More recently, AlbC from S. noursei (SEQ ID NO:1) and its homologue from S. albulus (99% sequence identity (238 amino acids identical/239 amino acids) and 100% sequence similarity over 239 residues) were shown to be able to form straight-chain dipeptides from one or more kinds of amino acids. A Patent Application (U.S. Patent Application No 20050287626) has been filed by Kyowa Hakko Kogyo Co.

The types of linear dipeptides that AlbC can produce has been reported as being combinations of phenylalanine, leucine and alanine.

The invention relates to a process to create a more diverse set of linear-chain dipeptides using cyclodipeptide synthases (CDSs), a new family of enzymes characterized by the Inventors and defined by the presence of a specific sequence signature. The Inventors have surprisingly found that AlbC from S. noursei and S. albulus is just one member of the CDS family and that the other members of the family identified by the Inventors in this application, display far lower, only 23-33% sequence identity with AlbC from S. noursei and 41-53% sequence similarity over 212-226 residues with AlbC from S. noursei.

The Inventors have also surprisingly found that the diverse members of the CDS family retain the required functionality to catalyse the synthesis of linear dipeptides and also surprisingly that these different members of the family exhibit a very useful diversity in the species of linear dipeptides which they can form, being able to catalyse the formation of linear dipeptides which are not formed by AlbC and that AlbC produces a far wider range of linear dipeptides than has been previously reported.

The Inventors provide the materials to carry out such a process and in particular provide the necessary nucleic acid and peptide sequences to code for the various CDS members they have identified, as well as vectors to genetically alter suitable microorganisms to express these enzymes.

The Inventors also provide the means to identify further members of this family using a variety of searching strategies, allowing further members to be isolated and characterized, further increasing the types of linear dipeptides which can be produced according to the current invention.

The invention relates to the use of an isolated, natural or synthetic protein or an active fragment of such a protein, selected in the group consisting of proteins or fragments thereof, having at least 20% identity and no more than 90% identity with SEQ ID NO:1, which corresponds to the AlbC protein from S. noursei. This protein or an active fragment of it has the ability to catalyse the formation of a linear dipeptide of the general formula (i):

R¹-R² (i)

(wherein R¹ and R², which may be the same or different, each represent any amino acid).

An active fragment of the protein is one which displays the ability to catalyse the formation of a linear dipeptide at statistically significant elevated level to the basal level of production for such substances. In particular an active fragment is considered to need to be at least seven amino acid residues in length to have functionality.

These percentages of sequence identity and sequence similarity defined herein were obtained using the BLAST program (blast2seq, default parameters) (Tatutsova and Madden, FEMS Microbiol Lett., 1999, 174, 247-250).

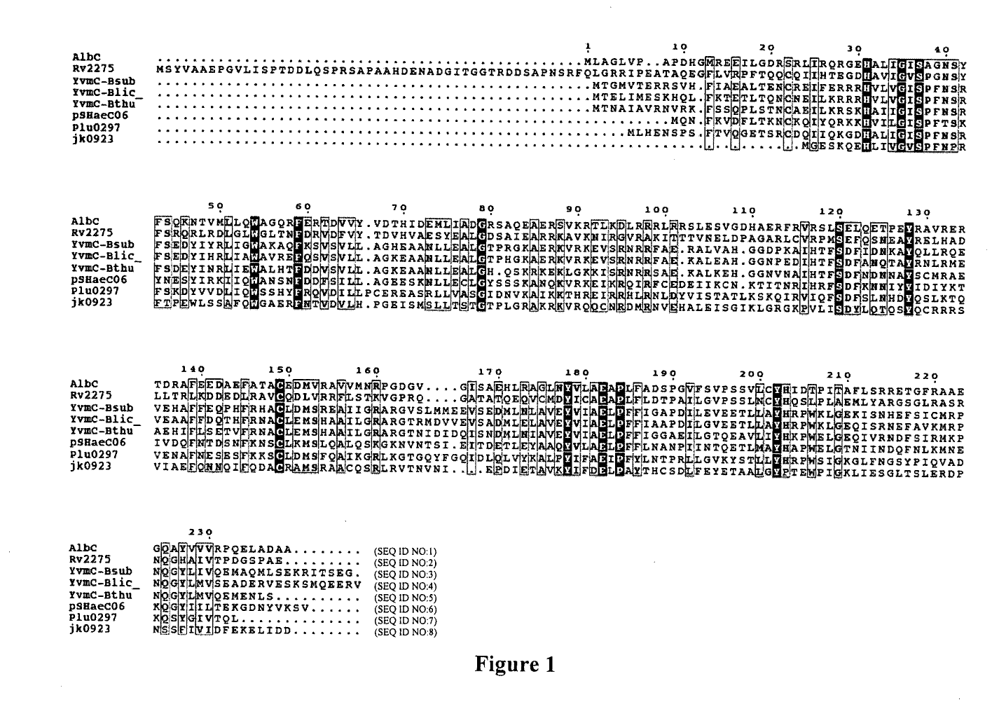

Such percentage sequence identity and similarity are derived from a full length comparison with SEQ ID NO:1, as shown in FIG. 1 herein; preferably these percentages are derived by calculating them on an overlap representing a percentage of length of said sequences as shown in FIG. 1.

Preferably the protein or an active fragment thereof has at least 20% and no more than 50% identity with SEQ ID NO:1.

Most preferably the protein or an active fragment thereof has at least 20% and no more than 35% identity with SEQ ID NO:1.

Comparison of the 239-amino acid sequence of AlbC, the first CDS described (Lautru et al., Chem. Biol., 2002, 9, 1355-1364), with databases led to the identification of seven hypothetical proteins of unknown function with moderate identity and similarity (FIG. 1). One 289-amino acid hypothetical protein that displays 33% identity and 53% similarity with AlbC over 212 residues was encoded by the genome of several organisms belonging to the Mycobacterium tuberculosis complex. This protein is named Rv2275 (SEQ ID NO:2) in Mycobacterium tuberculosis H37Rv (Acc n^{o} NP 216791), MT2335 in M. tuberculosis CDC 1551 (Acc n^{o} NP 336805), MRA2294 in M. tuberculosis H137Ra (Acc n^{o} YP001283620), TBFG12300 in M. tuberculosis F11 (Acc n^{o} YP001288233) and Mb2298 in Mycobacterium bovis AF2122/97 (Acc n^{o} NP 855947). Therefore, the protein encoded by several Mycobacteria strains will be called hereinafter Rv2275 (SEQ ID NO:2). Rv2275 is longer than AlbC and comprises a 49 amino acid N-terminal part that does not align with AlbC. Another hypothetical protein was found in M. bovis BCG strain Pasteur 1173P2. This protein named BCG2292 (Acc n^{o} YP978381 SEQ ID NO:34) is identical to the Rv2275 (SEQ ID NO:2) protein except that the E at residue 261 is replaced by A in SEQ ID NO:2.

Database searches also revealed three additional different homologous proteins originating from Bacillus species; two identical 249-amino acid hypothetical proteins named YvmC (hereinafter referred to as YvmC-Blic, SEQ ID NO:4) that present 29% identity and 47% similarity with AlbC over 221 residues were found in Bacillus licheniformis ATCC 14580 (Acc n^{o} AAU25020) and Bacillus licheniformis DSM 13 (Acc n^{o} AAU42391); one 248-amino acid YvmC (hereinafter referred to as YvmC-Bsub, SEQ ID NO:3) protein with 29% identity and 46% similarity with AlbC over 226 residues was encoded by Bacillus subtilis subsp. subtilis strain 168 (Acc. n^{o} CAB15512); one 238-amino acid hypothetical protein named RBTH_{—}07362 (hereinafter referred to as YvmC-Bthu, SEQ ID NO:5) that displays 26% identity and 45% similarity over 214 residues originated from Bacillus thuringiensis serovar israelensis ATCC 35646 (Acc n^{o} EA057133). In pair wise comparisons, these three different proteins from Bacillus species share higher sequence identity and similarity (61-70% identities and 76-81% similarities over 236-247 residues).

Among proteins homologous to AlbC also figured a 234-amino acid hypothetical protein Plu0297 (SEQ ID NO:7) that present 28% identity and 49% similarity with AlbC over 224 residues and that was found in Photorhabdus luminescens subsp. laumondii TTO1 (NP 927658).

Another AlbC homologous protein was encoded by the pSHaeC plasmid of about 8 kb harbored by the strain Staphylococcus haemolyticus JCSC1435; the protein named pSHaeC06 (SEQ ID NO:6) is 234-amino acid long and displays 20% identity and 44% similarity with AlbC over 220 amino acids (Acc n^{o} YP 254604).

Another hypothetical protein was found homologous to AlbC in the genome of Corynebacterium jeikeium K411; the 216-amino acid protein named Jk0923 (Acc n^{o} YP 250705, SEQ ID NO:8) presents 23% identity and 41% similarity over 212 residues with AlbC.

In all cases this correspondence occurs when the protein or an active fragment of this is compared to SEQ ID NO:1 using a pair wise comparison program such as BLAST to align these proteins or fragments thereof with SEQ ID NO:1 and allow the determination of where in upon SEQ ID NO:1 the conserved sequences appear.

The amino acid sequence alignment of AlbC with its seven related hypothetical proteins showed that only 13 positions are conserved among all proteins but it highlighted two particularly well-conserved regions, one comprising residues 31 to 37 (AlbC numbering) and the other one containing residues 178 to 184 (AlbC numbering) (FIG. 1).

These two regions were respectively used to define two sequence patterns, H-X-[LVI]-[LVI]-G-[LVI]-S (SEQ ID NO:9) and Y-[LVI]-X-X-E-X-P (SEQ ID NO:10), whose simultaneous presence in a protein when separated by 120-160 amino acids was scanned for in Uniprot (Nucleic Acids Res. 2007 January; 35(Database issue):D193-7.) using PATTINPROT (Combet et al., TIBS, 2000, 25, 147-150).

This search revealed only AlbC and its hereabove mentioned homologues (Rv2275 and BCG2292, YvmC-Bsub, Yvmc-Blic, YvmC-Bthu, Plu0297, pSHaeC06 and Jk0923). So, it has been shown that this first sequence signature can be used to search and define a new family of proteins related to AlbC; the Inventors have named all these enzymes cyclodipeptide synthases (CDSs). It has been shown below that the eight proteins belonging to this family are able to synthesize diverse linear dipeptides.

In a preferred embodiment of said use, the protein or an active fragment of it has a first conserved amino acid sequence of the general sequence SEQ ID NO:9:

| | |
|---|---|
| H-X-[LVI]-[LVI]-G-[LVI]-S, | (SEQ ID NO: 9) |

wherein H=histidine, X=any amino acid, [LVI]=any one of leucine, valine or isoleucine, G=glycine and S=serine.

In another preferred embodiment of said use, the protein or an active fragment of it has a second conserved amino acid sequence of the general sequence SEQ ID NO:10:

| | |
|---|---|
| Y-[LVI]-X-X-E-X-P, | (SEQ ID NO: 10) |

wherein Y=tyrosine, [LVI]=any one of leucine, valine or isoleucine, X=any amino acid, E=glutamic acid and P=proline.

Most preferably the protein or an active fragment of it has both the first and the second conserved amino acid sequences.

In another preferred embodiment of said use, the first conserved amino acid sequence and the second amino acid sequence are separated by at least 120 amino acid residues and no more than 160 amino acid residues.

Most preferably the first conserved amino acid sequence and the second amino acid sequence are separated by at least 140 amino acid residues and no more than 150 amino acid residues.

In another preferred embodiment of said use, the first conserved amino acid sequence corresponds to residues 31 to 37 of SEQ ID NO:1, in the protein or an active fragment of this.

In another preferred embodiment of said use, the second conserved amino acid sequence corresponds to residues 178 to 184 of SEQ ID NO:1 in the protein or an active fragment of it.

The Inventors have defined a new family of proteins related to AlbC, based on the presence of specified sequence signatures and similarities in size, they have now found that unexpectedly all members of the newly identified CDS family are also able to synthesize linear dipeptides.

In another preferred embodiment of said use, the protein or an active fragment of it, was isolated from a microorganism belonging to the genus Bacillus, Corynebacterium, Mycobacterium, Streptomyces, Photorhabdus or Staphylococcus.

According to a more preferred embodiment of said use, the protein or an active fragment of it, was isolated from a microorganism selected from the list Bacillus licheniformis, Bacillus subtilis subsp. subtilis, Bacillus thuringiensis serovar israelensis, Photorhabdus luminescens subsp. laumondii, Staphylococcus haemolyticus, Corynebacterium jeikeium, Mycobacterium tuberculosis, Mycobacterium bovis or Mycobacterium bovis BCG.

In another preferred embodiment of said use, the protein or an active fragment of it, is selected from the group consisting of AlbC (SEQ ID NO:1), Rv2275 (SEQ ID NO:2), MT2335 (SEQ ID NO:2), MRA2294 (SEQ ID NO:2), TBFG12300 (SEQ ID NO:2), Mb2298 (SEQ ID NO:2), BCG2292 (SEQ ID NO:34), YvmC-Bsub (SEQ ID NO:3), YvmC-Blic (SEQ ID NO:4), YvmC-Bthu (SEQ ID NO:5), pSHaeC06 (SEQ ID NO:6), Plu0297 (SEQ ID NO:7), JK0923 (SEQ ID NO:8), AlbC-his (SEQ ID NO:35), Rv2275-his (SEQ ID NO:36), YvmC-Bsub-his (SEQ ID NO:37).

Preferably the dipeptide may be in particular Phe-Leu, Leu-Phe, Phe-Phe, Phe-Tyr, Tyr-Phe, Leu-Leu, Leu-Tyr, Tyr-Leu, Phe-Met, Met-Phe, Leu-Met, Met-Leu, Tyr-Met, Met-Tyr, Met-Met, Tyr-Tyr, Ile-Met, Met-Ile, Ile-Leu.

The present invention also provides the use of an isolated, natural or synthetic nucleic acid sequence coding for a protein or an active fragment thereof, as specified herein.

The invention further relates to the use of a polynucleotide selected from:

a) a polynucleotide encoding a cyclodipeptide synthase as defined above;

b) a complementary polynucleotide of the polynucleotide a);

c) a polynucleotide which hybridizes to polynucleotide a) or b) under stringent conditions, for the synthesis of a linear dipeptide.

Advantageously, said polynucleotide is selected from the group consisting of the polynucleotides of sequences SEQ ID NO:11, SEQ ID NO:12, SEQ ID NO:13-16, 20 or 21. The polynucleotides of sequences SEQ ID NO:11, SEQ ID NO:12, SEQ ID NO:13-16 encode respectively the polypeptides of sequences SEQ ID NO:1-5 and SEQ ID NO:7, the polynucleotides SEQ ID NO:20 and 21 encode respectively the polypeptides of sequences SEQ ID NO:6 and 8; furthermore, the polynucleotide corresponding to positions 114-861 of SEQ ID NO:17 encodes the polypeptide AlbC-his of SEQ ID NO:35, the polynucleotide corresponding to positions 114-1008 of SEQ ID NO:18 encodes the polypeptide Rv2275-his of SEQ ID NO:36 and the polynucleotide corresponding to positions 114-885 of SEQ ID NO:19 encodes the polypeptide YvmC-Bsub-his of SEQ ID NO:37.

The term “hybridize(s)” as used herein refers to a process in which polynucleotides and/or oligonucleotides hybridize to the recited nucleic acid sequence or parts thereof. Therefore, said nucleic acid sequence may be useful as probes in Northern or Southern Blot analysis of RNA or DNA preparations, respectively, or can be used as oligonucleotide primers in PCR analysis dependent on their respective size. Preferably, said hybridizing oligonucleotides comprise at least 10 and more preferably at least 15 nucleotides. While a hybridizing polynucleotide of the present invention to be used as a probe preferably comprises at least 100 and more preferably at least 200, or most preferably at least 500 nucleotides.

It is well known in the art how to perform hybridization experiments with nucleic acid molecules, i.e. the person skilled in the art knows what hybridization conditions she/he has to use in accordance with the present invention. Such hybridization conditions are referred to in standard text books such as Sambrook et al., Molecular Cloning: A Laboratory Manual; Cold Spring Harbor Laboratory Press, 2^{nd} edition 1989 and 3^{rd} edition 2001; Gerhardt et al.; Methods for General and Molecular Bacteriology; ASM Press, 1994; Lefkovits; Immunology Methods Manual: The Comprehensive Sourcebook of Techniques; Academic Press, 1997; Golèmis; Protein-Protein Interactions: A Molecular Cloning Manual; Cold Spring Harbor Laboratory Press, 2002 and other standard laboratory manuals known by the person skilled in the Art or as recited above. Preferred in accordance with the present inventions are stringent hybridization conditions.

“Stringent hybridization conditions” refer, e.g. to an overnight incubation at 42° C. in a solution comprising 50% formamide, 5×SSC (750 mM NaCl, 75 mM sodium citrate), 50 mM sodium phosphate (pH 7.6), 5×Denhardt's solution, 10% dextran sulfate, and 20 μg/ml denatured, sheared salmon sperm DNA, followed e.g. by washing the filters in 0.2×SSC at about 65° C.

Also contemplated are nucleic acid molecules that hybridize at low stringency hybridization conditions. Changes in the stringency of hybridization and signal detection are primarily accomplished through the manipulation of formamide concentration; salt conditions, or temperature. For example, lower stringency conditions include an overnight incubation at 37° C. in a solution comprising 6×SSPE (20×SSPE=3 mol/l NaCl; 0.2 mol/l NaH₂PO₄; 0.02 mol/l EDTA, pH 7.4), 0.5% SDS, 30% formamide, 100 μg/ml salmon sperm blocking DNA; followed by washes at 50° C. with 1×SSPE, 0.1% SDS.

In addition, to achieve even lower stringency, washes performed following stringent hybridization can be done at higher salt concentrations (e.g. 5×SSC). It is of note that variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations.

The present invention also provides a recombinant vector comprising a nucleic acid coding sequence as defined hereabove. This vector is configured to introduce the nucleic acid coding sequence into a host cell and this coding sequence is thereby transcribed and translated by the endogenous transcription and translation mechanisms of the host cell.

The recombinant vector may comprise coding sequences for at least two proteins or active fragments thereof as defined hereabove. By providing multiple coding sequences the Inventors provide a means of producing several enzyme specific linear dipeptides, by including suitable coding sequences from several such CDS enzymes.

Hence, the at least two coding sequences come from different genes.

Alternatively the at least two coding sequences come from a single gene. In such a case the provision of multiple coding sequences for the same gene product allows the amplification of the exogenous gene product levels so increasing the rate of linear dipeptide formation.

Preferably the host cell is a prokaryote. Prokaryotic cells are generally simple to culture and easily stored between rounds of fermentation, making them an ideal system in which to produce on a large scale significant levels of linear dipeptide from simple media and growing conditions.

Most preferably the host cell is Escherichia coli, the best characterized prokaryotic organism in which a plurality of different expression systems and culture technologies exist.

The present invention further relates to a recombinant vector comprising said nucleic acid coding sequence as defined hereabove. This vector is configured to express the nucleic acid coding sequence in a cell free expression system by the endogenous mechanisms of this cell free expression system.

The present invention also provides a method for the production of a linear dipeptide, comprising the steps:

a) culturing upon a medium a host cell which has the ability to produce a protein or an active fragment thereof having the activity to form a linear dipeptide from one or more kinds of amino acids;

b) allowing the linear dipeptide to form and accumulate in the host cell and in some cases also in the medium;

c) recovering the linear dipeptide from the cellular extract and medium;

wherein the protein or an active fragment thereof is selected in the group consisting of proteins and fragments thereof, having at least 20% identity and no more than 90% identity with SEQ ID NO:1.

Preferably the protein or an active fragment thereof is also encoded by an endogenous gene of the host cell.

Alternatively the protein or an active fragment thereof is not encoded by an endogenous gene of said host cell.

The present invention relates also to a method for the production of a linear dipeptide, comprising the steps:

a) inducing a cell free expression system to produce a protein or an active fragment thereof, having the activity to form a linear dipeptide from one or more kinds of amino acids;

b) introducing at least one amino acid substrate to the protein or an active fragment thereof;

c) allowing the linear dipeptide to form and accumulate;

d) recovering the linear dipeptide;

wherein the protein or an active fragment thereof is selected in the group consisting of proteins and fragments thereof, having at least 20% identity and no more than 90% identity with SEQ ID NO:1.

The present invention further provides a method of identifying polypeptides that catalyse the formation of a linear dipeptide of the general formula (i):

R¹-R² (i)

(wherein R¹ and R², which may be the same or different and each may represent any amino acid);

characterised in that it comprises the steps:

a) identifying a candidate polypeptide sequence as having at least one of the following motifs:

| | |
|---|---|
| H-X-[LVI]-[LVI]-G-[LVI]-S | (SEQ ID NO: 9) |

wherein H=histidine, X=any amino acid, [LVI]=any one of leucine, valine or isoleucine, G=glycine and S=serine; and
wherein at least one of said H, LVI, G or S can be another amino acid namely H can be replaced by any one of Lysine or Arginine; LVI can be replaced by any one of Glycine, Alanine, Leucine, Valine or Isoleucine; G can be replaced by any one of Glycine, Alanine, Leucine, Valine or Isoleucine; S can be replaced by Cysteine, Threonine or Methionine.

| | |
|---|---|
| Y-[LVI]-X-X-E-X-P | (SEQ ID NO: 10) |

wherein Y=tyrosine, [LVI]=any one of leucine, valine or isoleucine, X=any amino acid, E=glutamic acid and P=proline; and
wherein at least one of said Y, LVI, E, X or P can be another amino acid namely Y can be replaced by any one of Phenylalanine or Trytophan; LVI can be replaced by any one of Glycine, Alanine, Leucine, Valine or Isoleucine; E can be replaced by any one of Aspartic Acid, Asparagine, Glutamine; P can be replaced by any one of Glycine, Alanine, Leucine, Valine or Isoleucine;

b) creating a polypeptide expression construct by linking said candidate polypeptide coding sequence to promoter sequences configured to express said candidate peptide at an appreciable level;

c) introducing said polypeptide expression construct into at least one cell and inducing the take up of said polypeptide expression construct by said at least one cell or a cell free expression system;

d) monitoring the levels and types of linear dipeptides in the growth medium of said at least one cell or said cell free expression system;

e) comparing the levels of linear dipeptides in the presence of said polypeptide expression construct to the levels of linear dipeptides in the absence of said polypeptide expression construct to determine the relative level of production of linear dipeptides by said polypeptide expression construct; and

f) correlating the relative production of linear dipeptides to expression of said candidate polypeptide in said at least one cell or said cell free expression system.

The Inventors therefore provide a systematic approach to the identification of further enzymes capable of synthesizing linear dipeptides. This approach uses the two conserved motifs which the Inventors have identified for the first time and allows the identification of suitable candidate polypeptides in silico which have one or both of these domains or derivatives thereof.

These candidate polypeptides are then linked to a suitable promoter, whose properties allow the expression of the candidate polypeptide at a level where its activity becomes appreciable. The exact level required to become appreciable will vary depending upon the exact expression system used and as such specific details are not provided by the Inventors as this is a common experimental practice.

According to a preferred embodiment of said method, the said first conserved motif (SEQ ID NO:9) and the second conserved motif (SEQ ID NO:10) are separated by at least 75 and no more than 250 amino acids.

The identification system for candidate polypeptides may also therefore encompass candidate molecules in which the first and second conserved motifs (SEQ ID NO:9 and 10 respectively) where both present are separated by a variable stretch of 75 and 250 amino acids.

Preferably the first conserved motif (SEQ ID NO:9) and/or the second conserved motif (SEQ ID NO:10) comprise more than one residue change.

The present invention also provides a method of identifying polypeptides that catalyse the formation of a linear dipeptide of the general formula (i):

R¹-R² (i)

(wherein R¹ and R², which may be the same or different and each may represent any amino acid);

characterized in that it comprises the steps:

a) identifying a candidate polypeptide sequence as having at least 20% identity and no more than 90% identity with SEQ ID NO:1; or having at least 20% identity with any one of SEQ ID NO:2, SEQ ID NO:3, SEQ ID NO:4, SEQ ID NO:5, SEQ ID NO:6, SEQ ID NO:7, SEQ ID NO:8, SEQ ID NO:35, SEQ ID NO:36, SEQ ID NO:37;

b) creating a polypeptide expression construct by linking the candidate polypeptide sequence to promoter sequences configured to express said candidate peptide at an appreciable level;

c) introducing the polypeptide expression construct into at least one cell or a cell free expression system and inducing the expression of the polypeptide expression construct by the at least one cell or cell free expression system;

d) monitoring the levels and types of linear dipeptides in the cellular extract and growth medium of the at least one cell or the cell free expression system;

e) comparing the levels of linear dipeptides in the presence of the polypeptide expression construct to the levels of linear dipeptides in the absence of the polypeptide expression construct to determine the relative level of production of linear dipeptides by the polypeptide fusion construct; and

f) correlating the relative production of linear dipeptides to the expression of the candidate polypeptide in said at least one cell or the cell free expression system.

For a better understanding of the invention and to show how the same may be carried into effect, there will now be shown by way of example only, specific embodiments, methods and processes according to the present invention with reference to the accompanying drawings in which:

FIG. 1 illustrates the amino acid sequence alignment of AlbC (SEQ ID NO:1) from Streptomyces noursei with other CDS proteins. The related proteins are Rv2275 (SEQ ID NO:2) from Mycobacterium tuberculosis, YvmC from Bacillus subtilis (herein referred to as YvmC-Bsub, SEQ ID NO:3), YvmC from Bacillus licheniformis (herein referred to as YvmC-Blic, SEQ ID NO:4), YvmC from Bacillus thuringiensis (herein referred to as YvmC-Bthu, SEQ ID NO:5), pSHaeC06 (SEQ ID NO:6) from Staphylococcus haemolyticus, Plu0297 (SEQ ID NO:7) from Photorhabdus luninescens and Jk0923 (SEQ ID NO:8) from Corynebacterium jeikeium. The thirteen positions highly conserved (identical residue in all sequences) are indicated by a black background. Positions with moderate conservation are boxed.

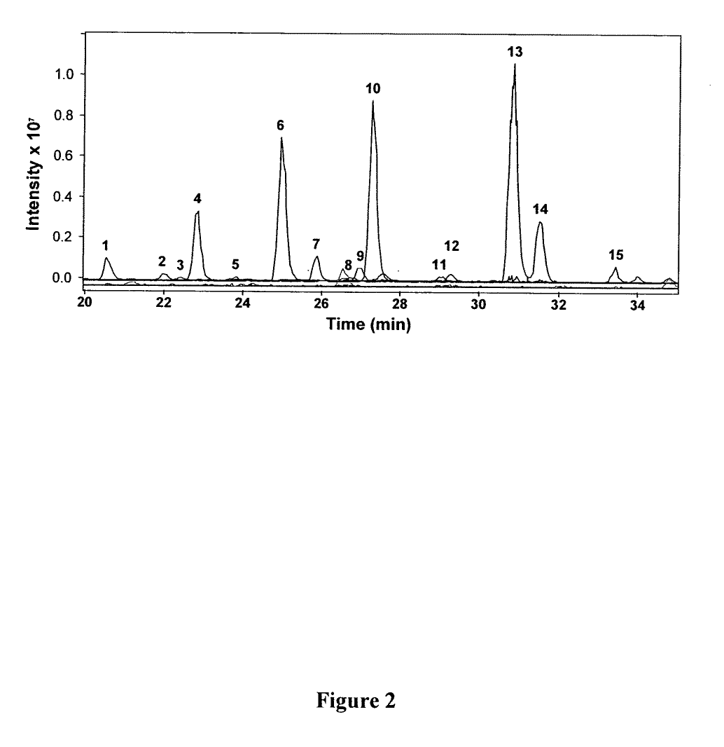

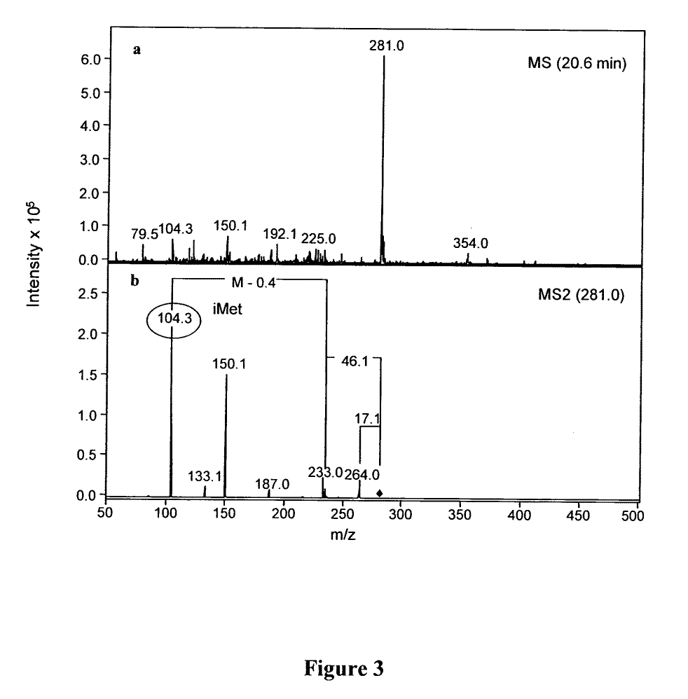

FIG. 2 illustrates EICs of dipeptides m/z values specific to AlbC-his (SEQ ID NO:35) and detected from a LC-MS analysis of the soluble fraction of E. coli cells expressing AlbC-his (upper black traces) compared to the same set of EICs from a LCMS analysis of the control sample (lower grey traces). Each specific EIC peak was labeled as specified in Table II for identification by MS and MS/MS illustrated in the FIGS. 3 to 17.

FIG. 3 illustrates the MS and MS/MS spectra of the EIC peak 1 detected at 20.6 min during the analysis of the soluble fraction of E. coli cells expressing AlbC.

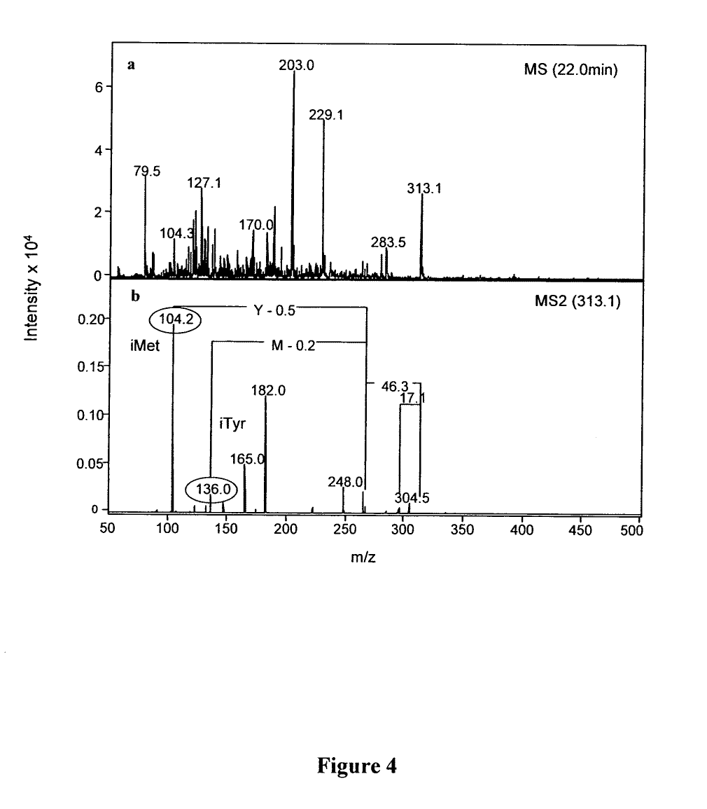

FIG. 4 illustrates the MS and MS/MS spectra of the EIC peak 2 detected at 22.0 min during the analysis of the soluble fraction of E. coli cells expressing AlbC.

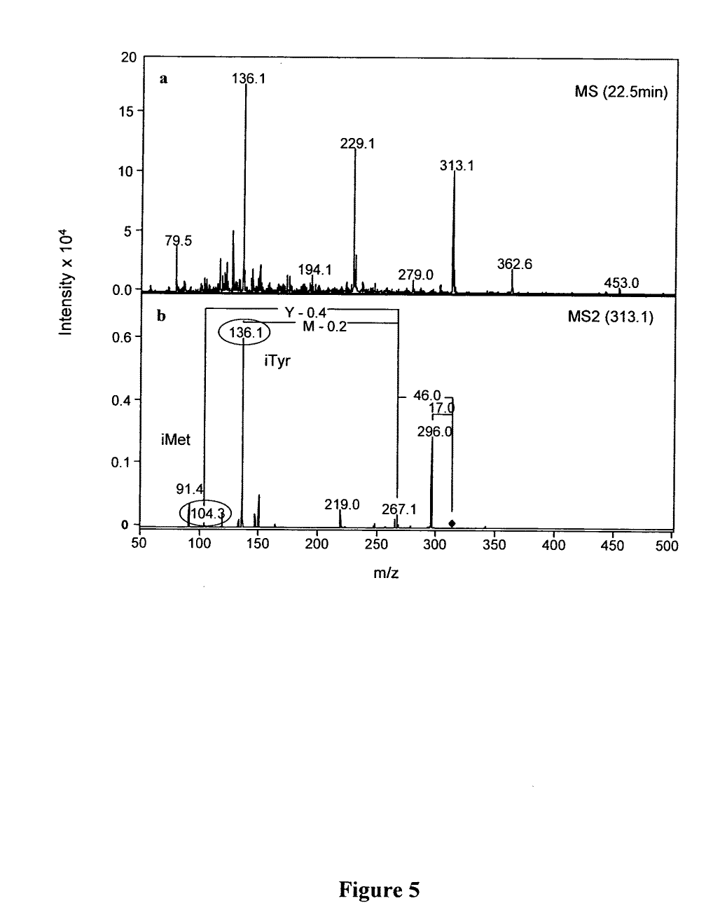

FIG. 5 illustrates the MS and MS/MS spectra of the EIC peak 3 detected at 22.5 min during the analysis of the soluble fraction of E. coli cells expressing AlbC.

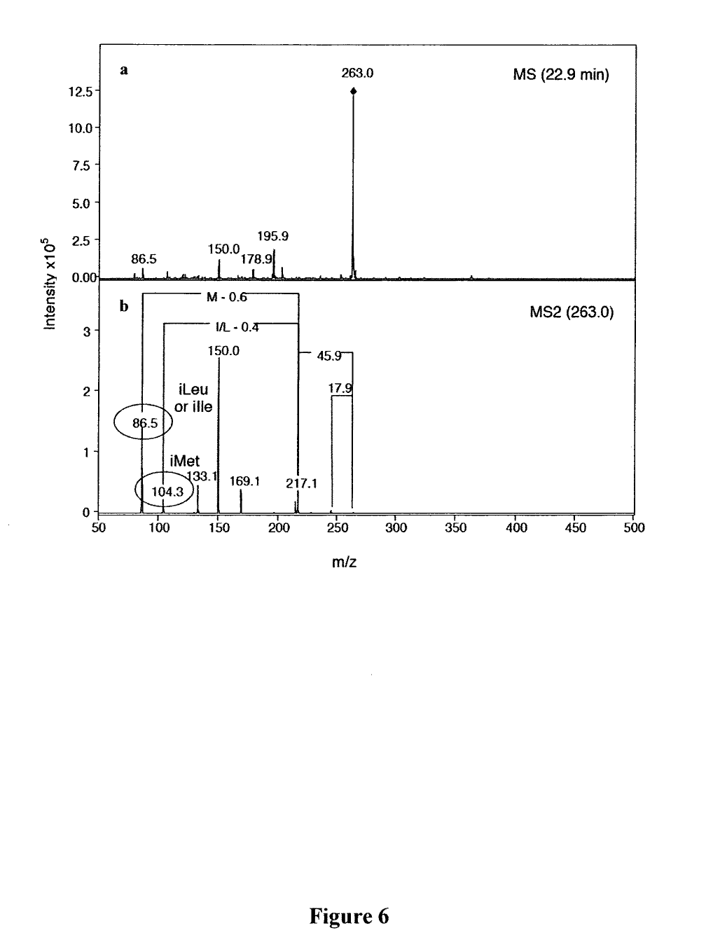

FIG. 6 illustrates the MS and MS/MS spectra of the EIC peak 4 detected at 22.9 min during the analysis of the soluble fraction of E. coli cells expressing AlbC.

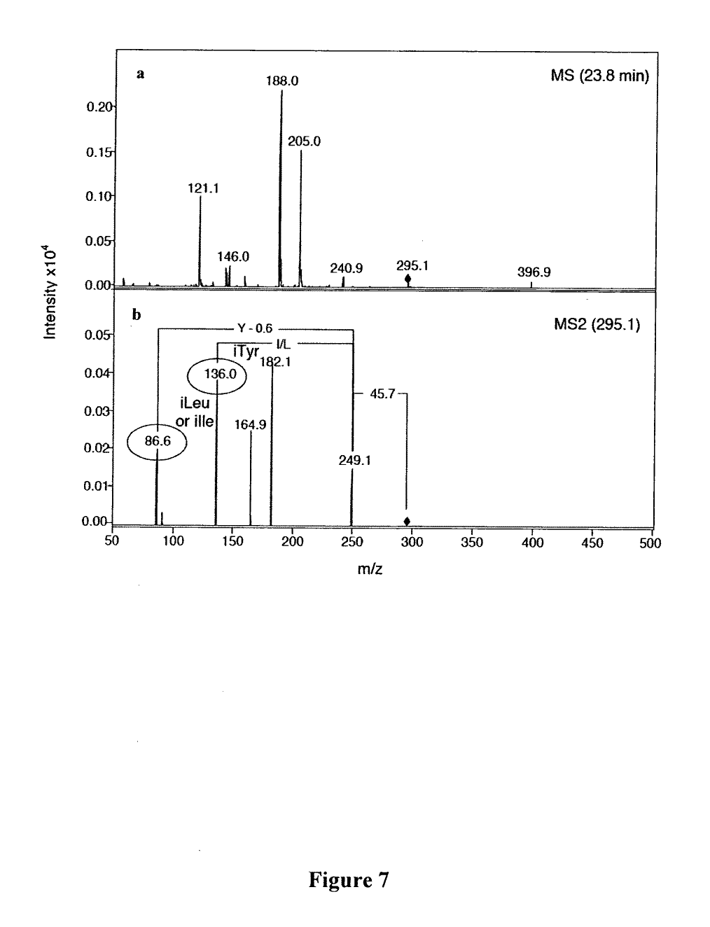

FIG. 7 illustrates the MS and MS/MS spectra of the EIC peak 5 detected at 23.8 min during the analysis of the soluble fraction of E. coli cells expressing AlbC.

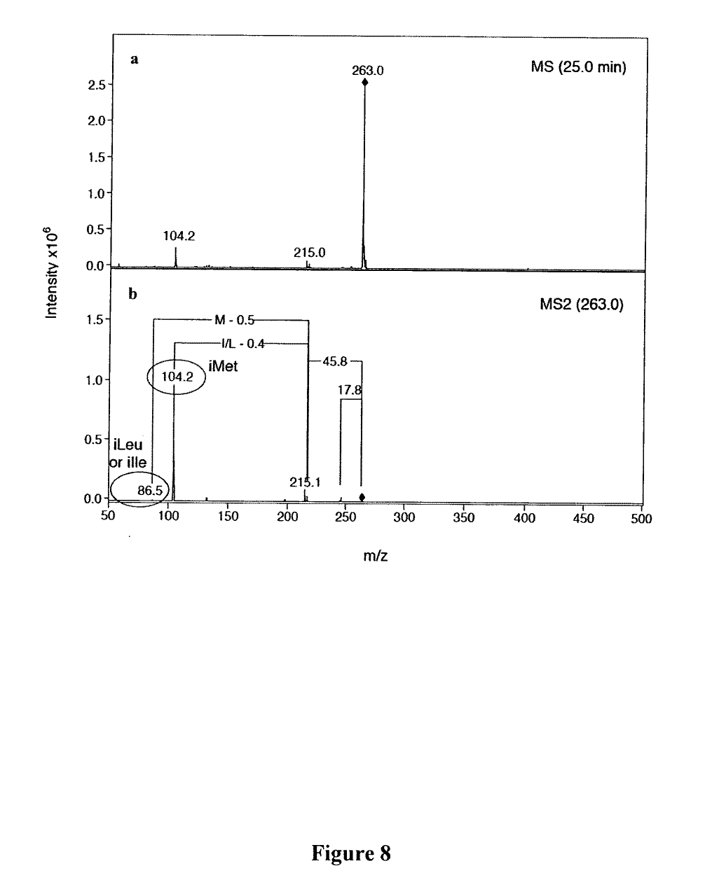

FIG. 8 illustrates the MS and MS/MS spectra of the EIC peak 6 detected at 25.0 min during the analysis of the soluble fraction of E. coli cells expressing AlbC.

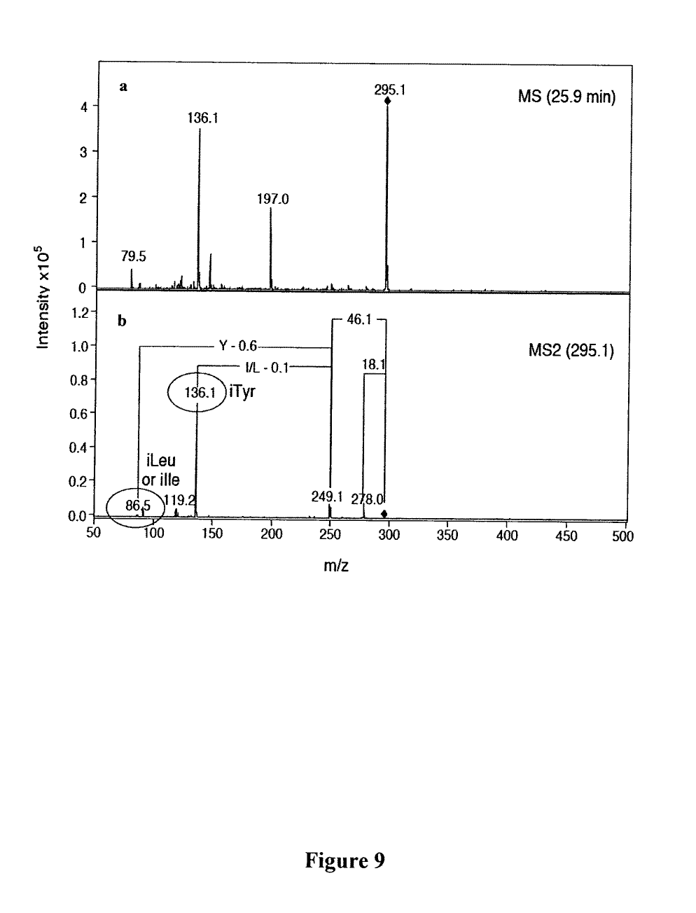

FIG. 9 illustrates the MS and MS/MS spectra of the EIC peak 7 detected at 25.9 min during the analysis of the soluble fraction of E. coli cells expressing AlbC.

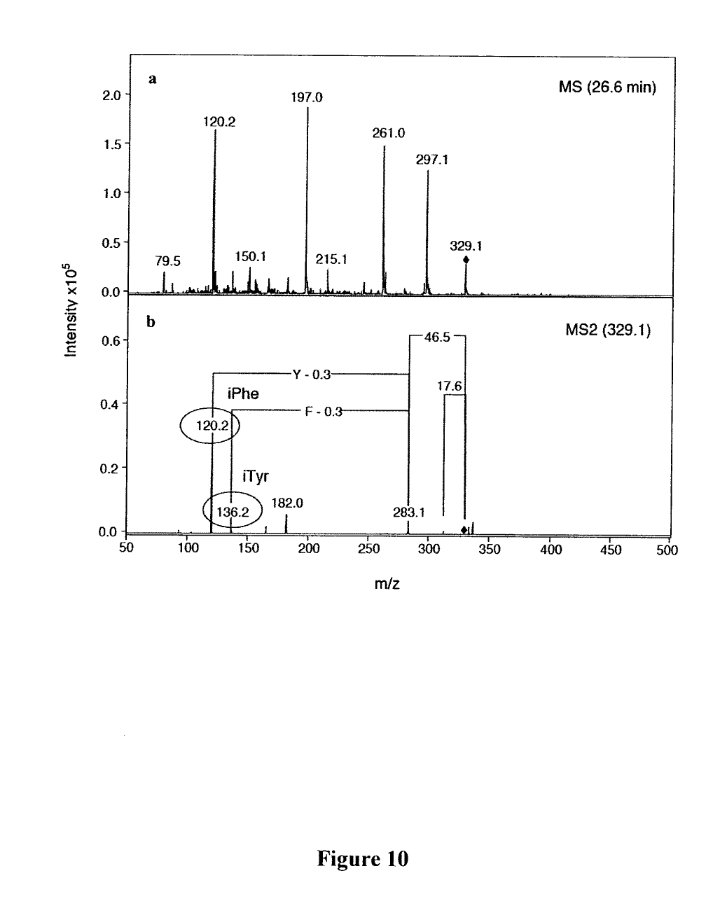

FIG. 10 illustrates the MS and MS/MS spectra of the EIC peak 8 detected at 26.6 min during the analysis of the soluble fraction of E. coli cells expressing AlbC.

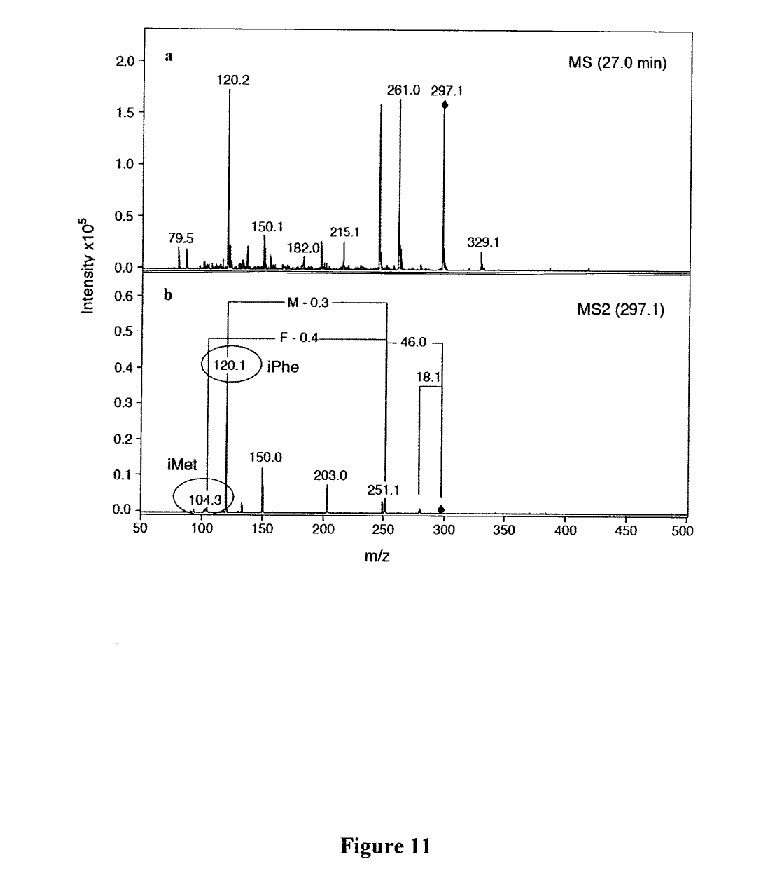

FIG. 11 illustrates the MS and MS/MS spectra of the EIC peak 9 detected at 27.0 min during the analysis of the soluble fraction of E. coli cells expressing AlbC.

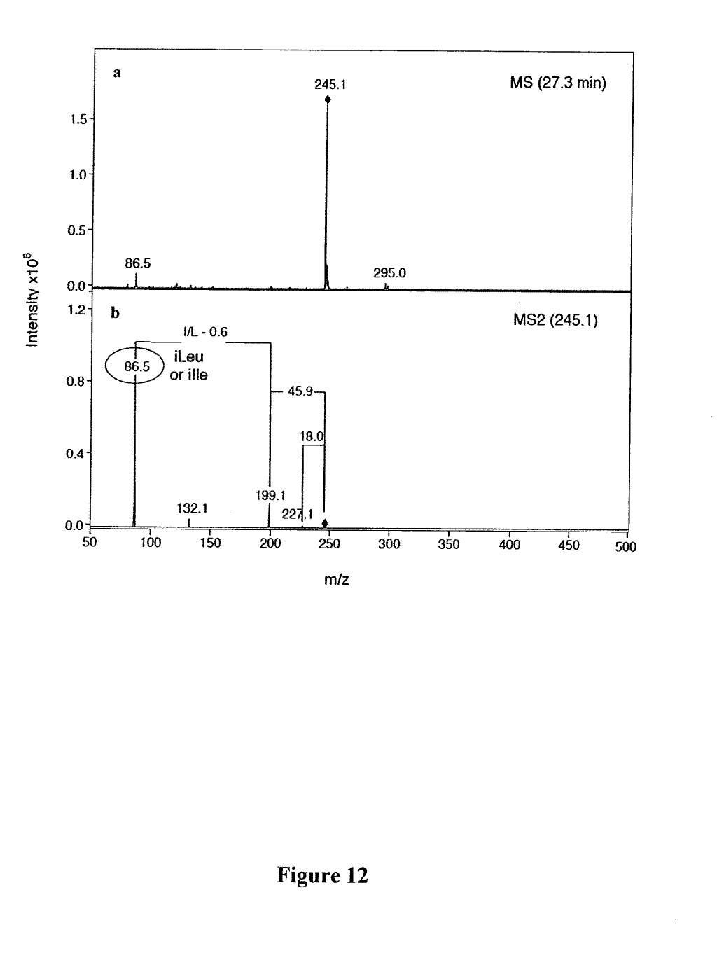

FIG. 12 illustrates the MS and MS/MS spectra of the EIC peak 10 detected at 27.3 min during the analysis of the soluble fraction of E. coli cells expressing AlbC.

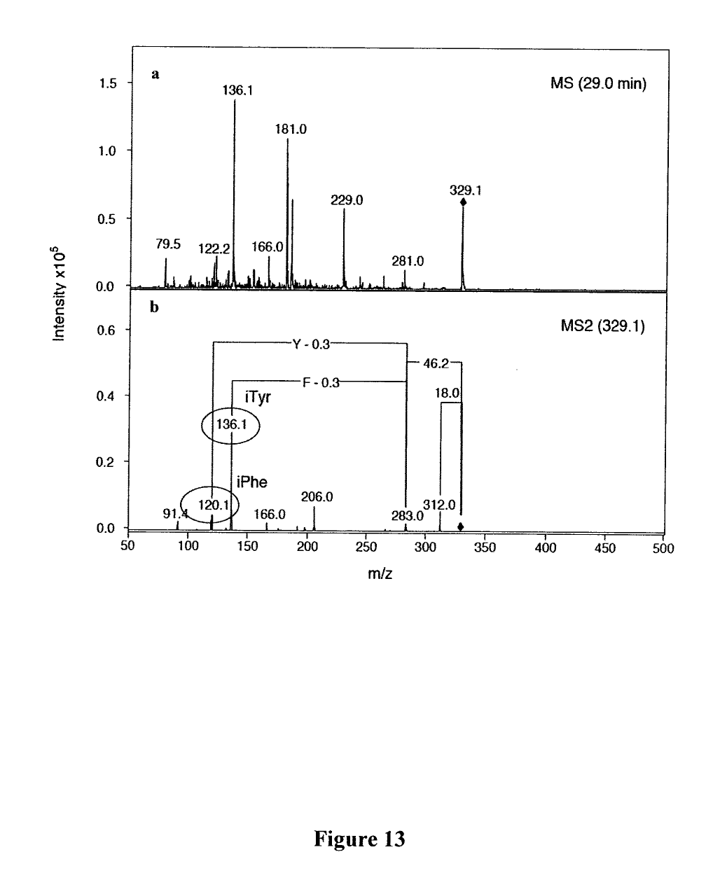

FIG. 13 illustrates the MS and MS/MS spectra of the EIC peak 11 detected at 29.0 min during the analysis of the soluble fraction of E. coli cells expressing AlbC.

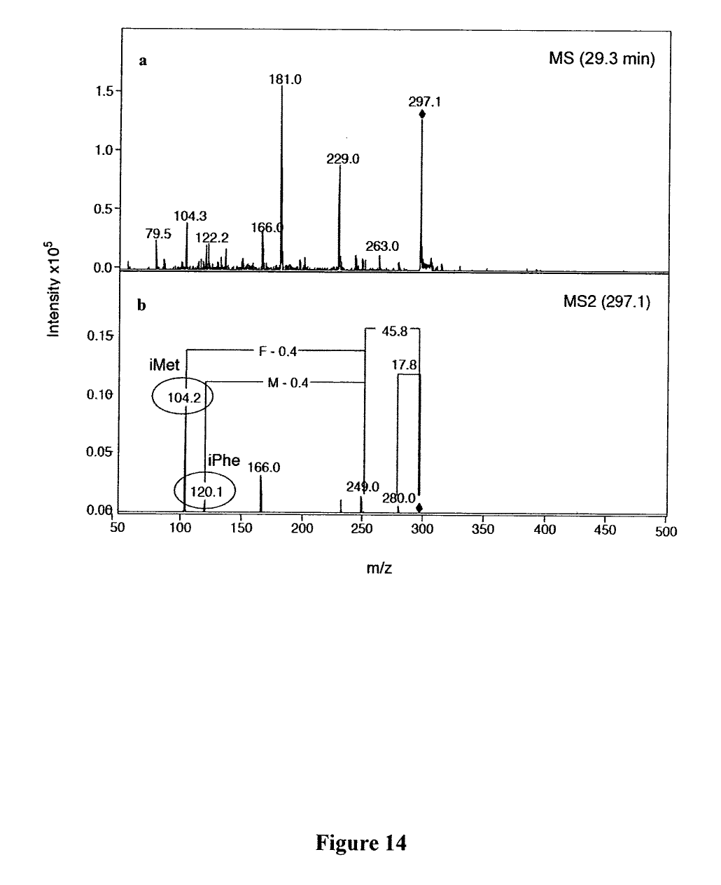

FIG. 14 illustrates the MS and MS/MS spectra of the EIC peak 12 detected at 29.3 min during the analysis of the soluble fraction of E. coli cells expressing AlbC.

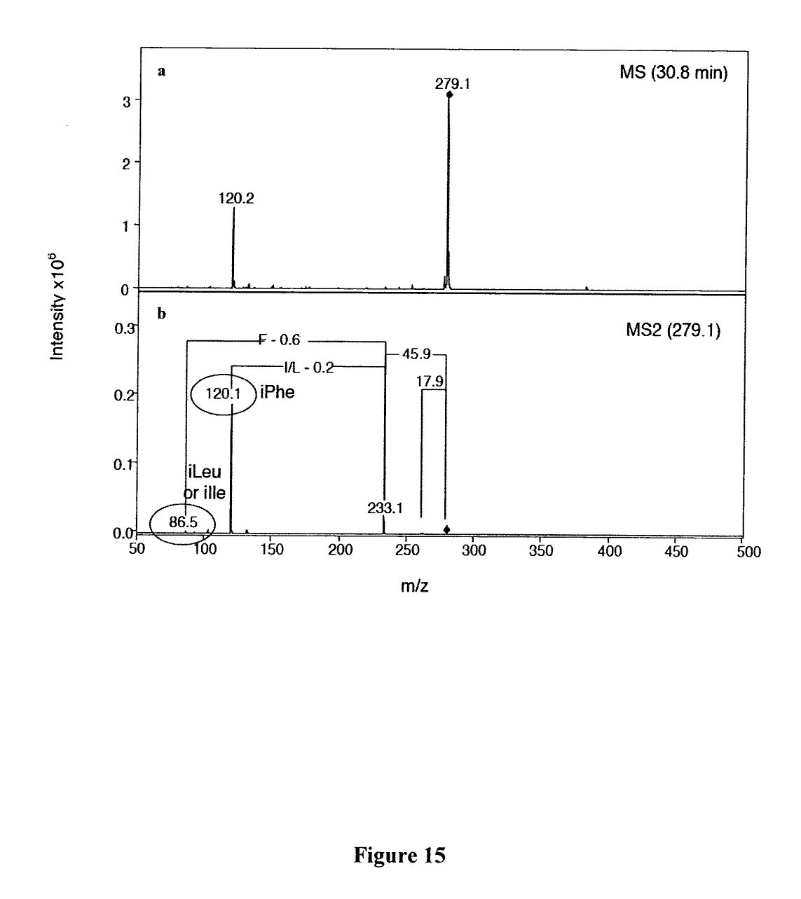

FIG. 15 illustrates the MS and MS/MS spectra of the EIC peak 13 detected at 30.8 min during the analysis of the soluble fraction of E. coli cells expressing AlbC.

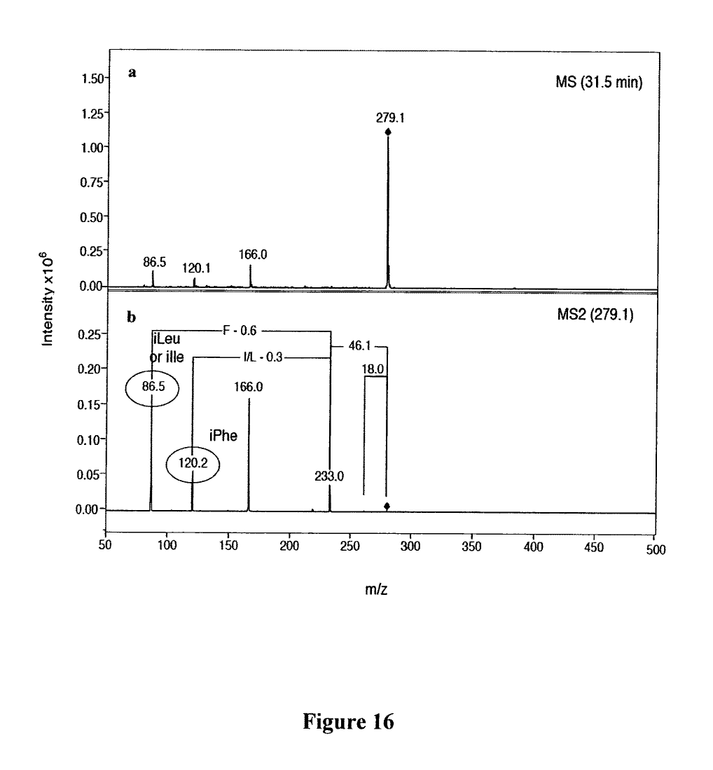

FIG. 16 illustrates the MS and MS/MS spectra of the EIC peak 14 detected at 31.5 min during the analysis of the soluble fraction of E. coli cells expressing AlbC.

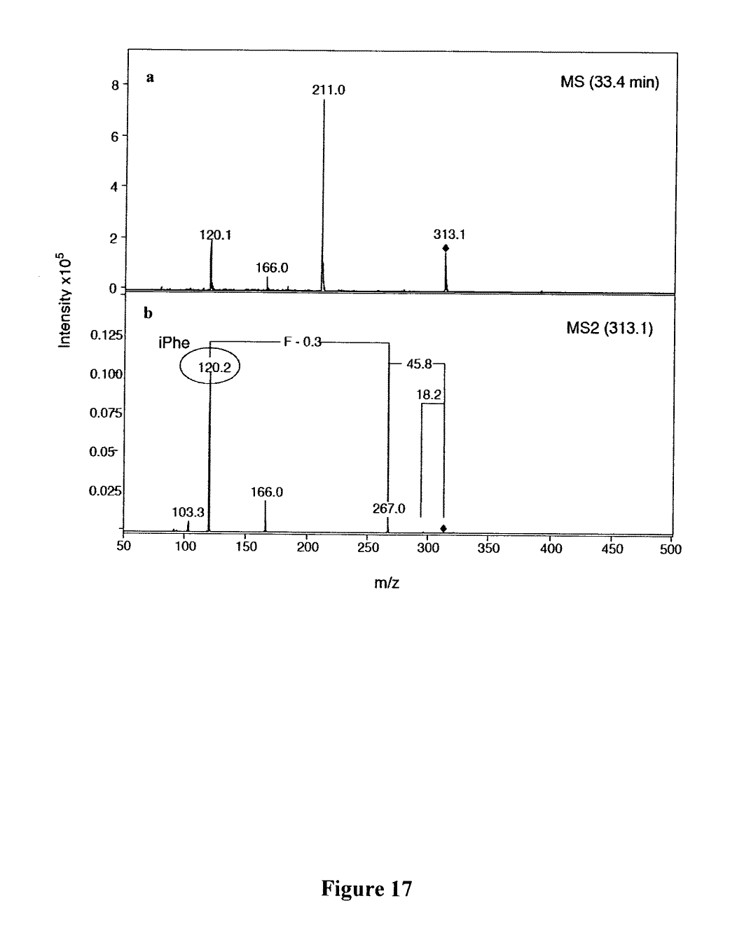

FIG. 17 illustrates the MS and MS/MS spectra of the EIC peak 15 detected at 33.4 min during the analysis of the soluble fraction of E. coli cells expressing AlbC.

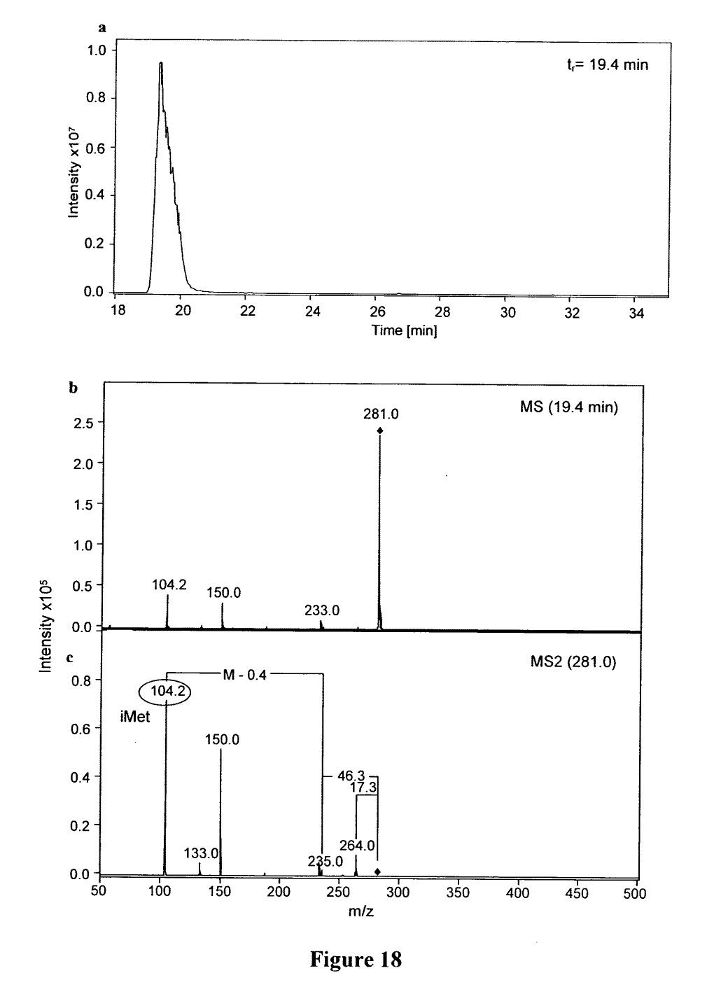

FIG. 18 illustrates the EIC and the MS and MS/MS spectra of the chemically-synthesized Met-Met. An EIC peak is detected at 19.4 minutes (FIG. 18a).

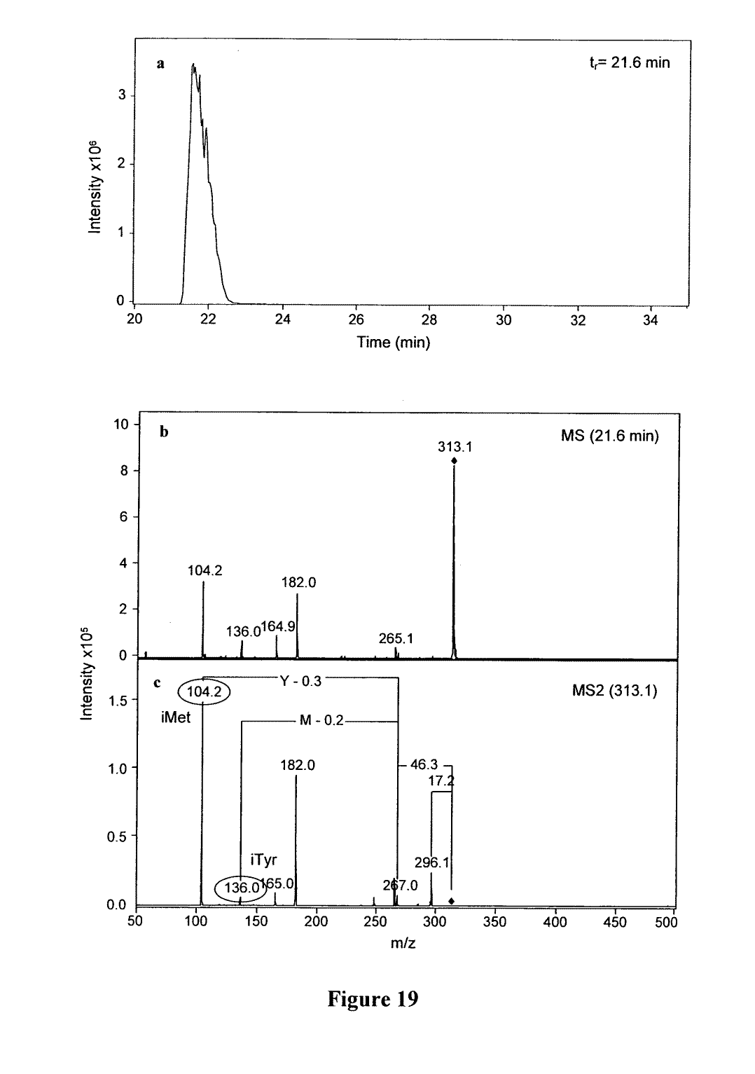

FIG. 19 illustrates the EIC and the MS and MS/MS spectra of the chemically-synthesized Met-Tyr. An EIC peak is detected at 21.6 minutes (FIG. 19a).

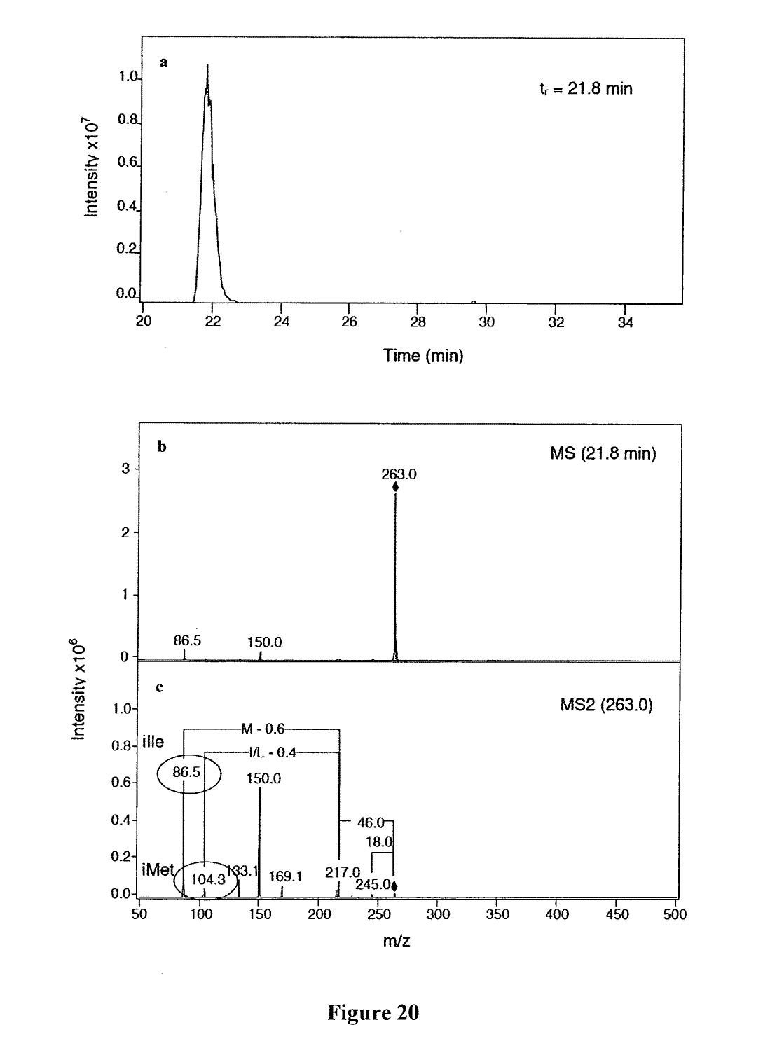

FIG. 20 illustrates the EIC and the MS and MS/MS spectra of the chemically-synthesized Ile-Met. An EIC peak is detected at 21.8 minutes (FIG. 20a).

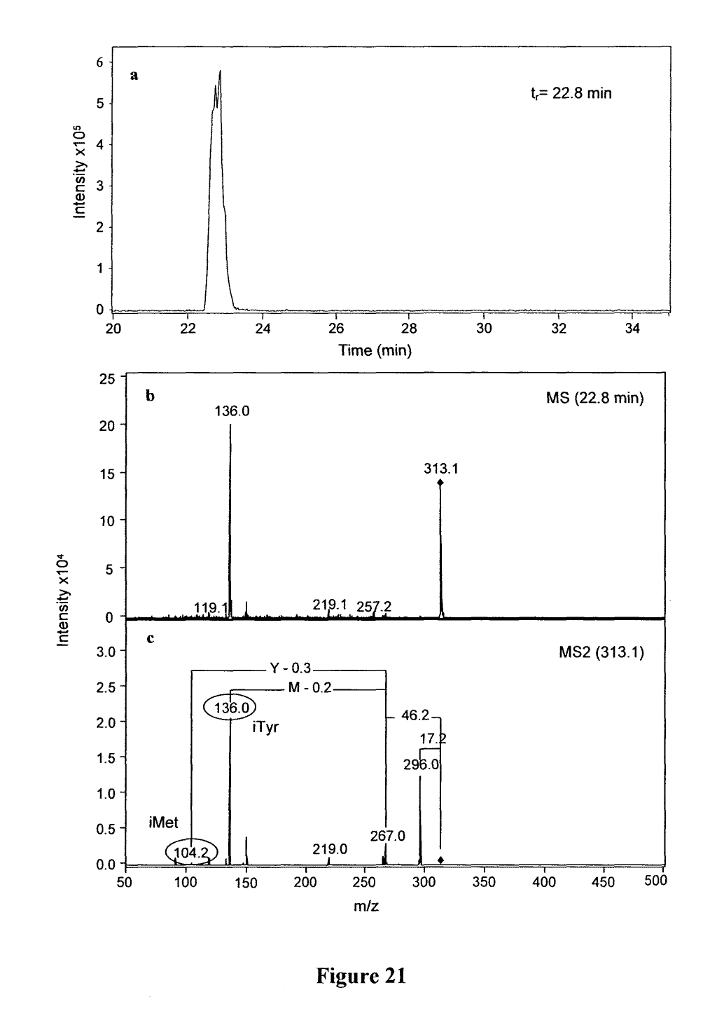

FIG. 21 illustrates the EIC and the MS and MS/MS spectra of the chemically-synthesized Tyr-Met. An EIC peak is detected at 22.8 minutes (FIG. 21a).

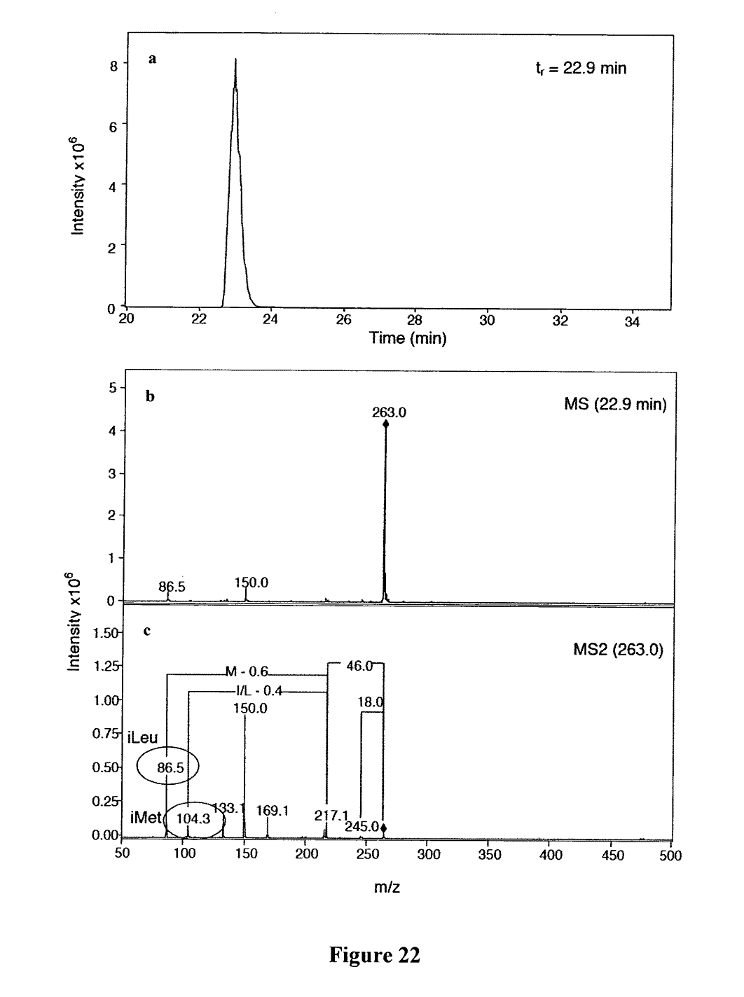

FIG. 22 illustrates the EIC and the MS and MS/MS spectra of the chemically-synthesized Leu-Met. An EIC peak is detected at 22.9 minutes (FIG. 22a).

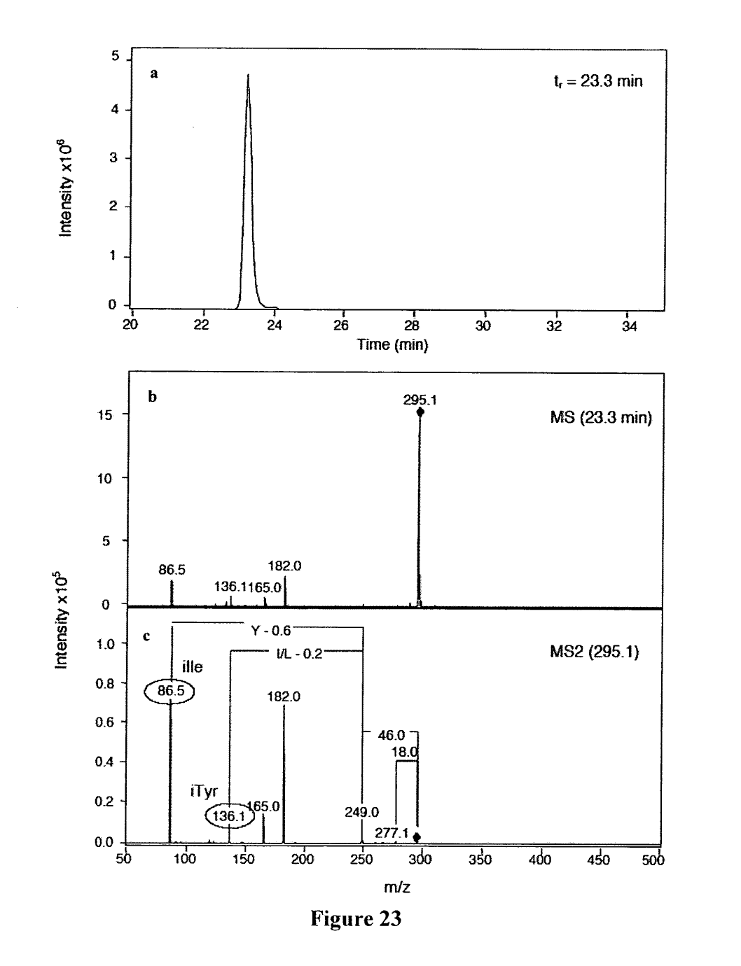

FIG. 23 illustrates the EIC and the MS and MS/MS spectra of the chemically-synthesized Ile-Tyr. An EIC peak is detected at 23.3 minutes (FIG. 23a).

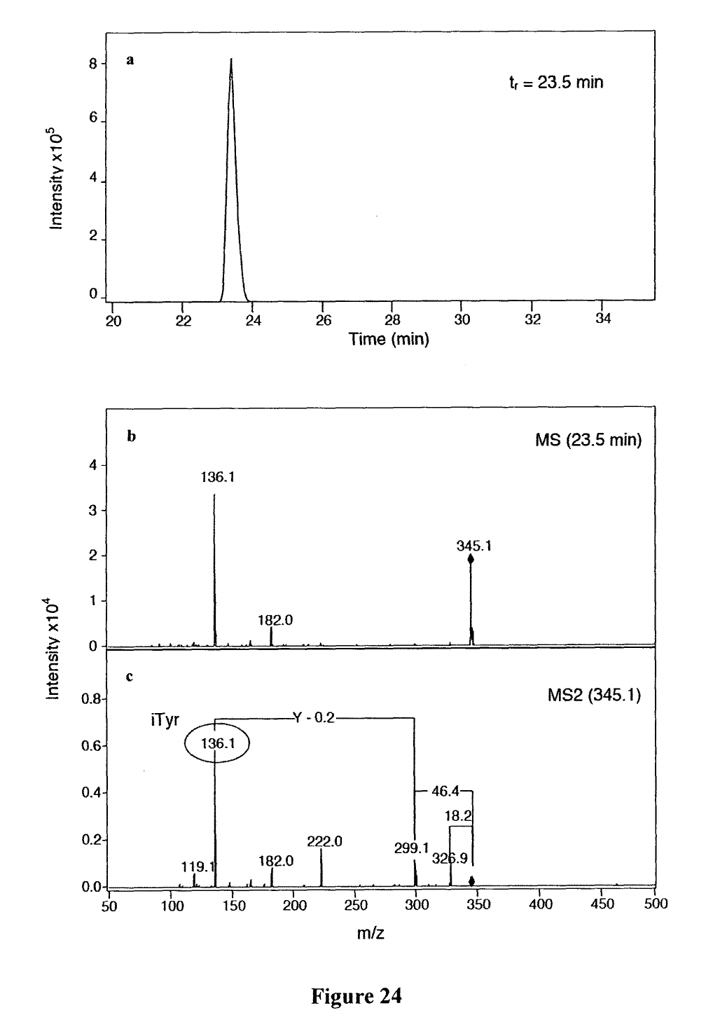

FIG. 24 illustrates the EIC and the MS and MS/MS spectra of the chemically-synthesized Tyr-Tyr. An EIC peak is detected at 23.5 minutes (FIG. 24a).

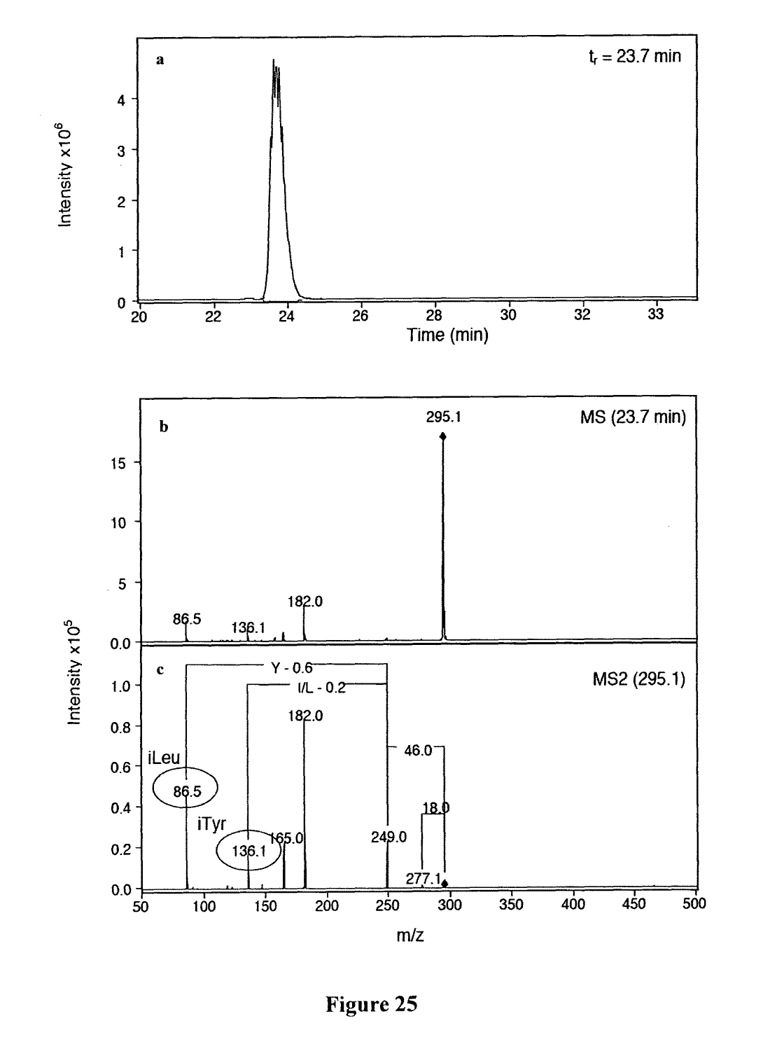

FIG. 25 illustrates the EIC and the MS and MS/MS spectra of the chemically-synthesized Leu-Tyr. An EIC peak is detected at 23.7 minutes (FIG. 25a).

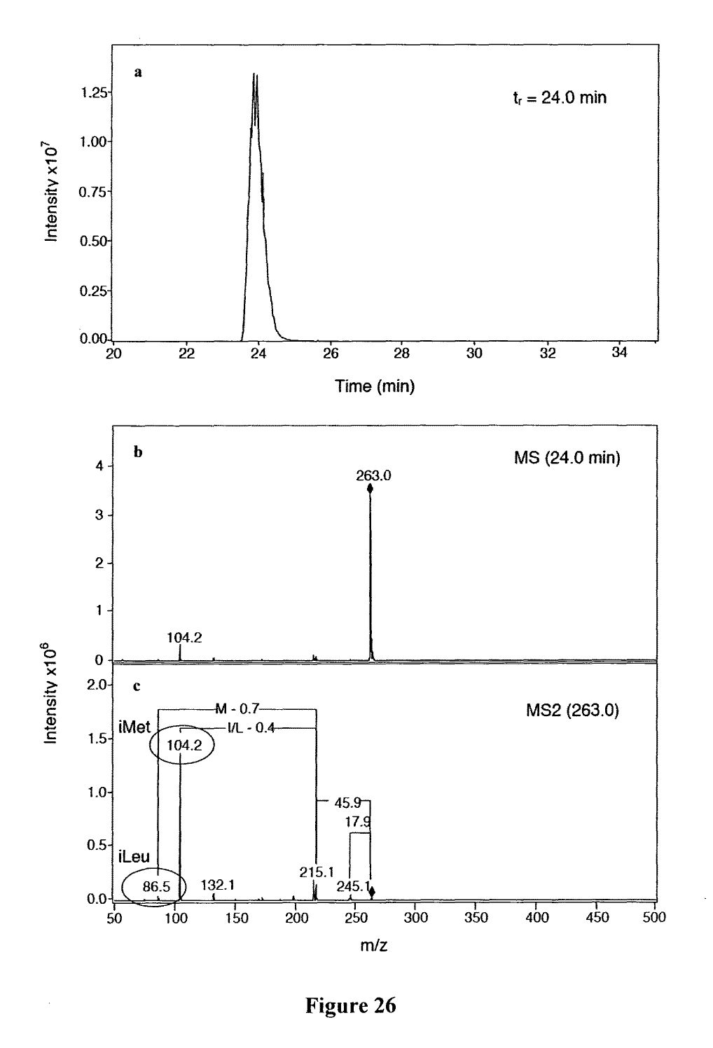

FIG. 26 illustrates the EIC and the MS and MS/MS spectra of the chemically-synthesized Met-Ile. An EIC peak is detected at 24.0 minutes (FIG. 26a).

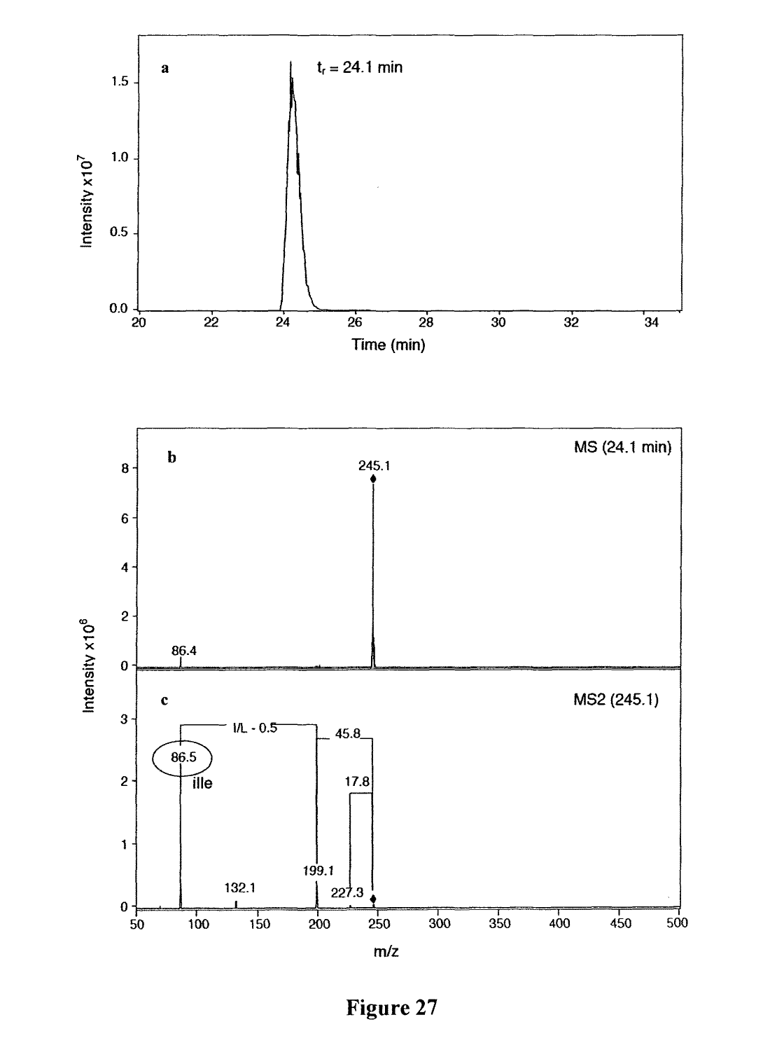

FIG. 27 illustrates the EIC and the MS and MS/MS spectra of the chemically-synthesized Ile-Ile. An EIC peak is detected at 24.1 minutes (FIG. 27a).

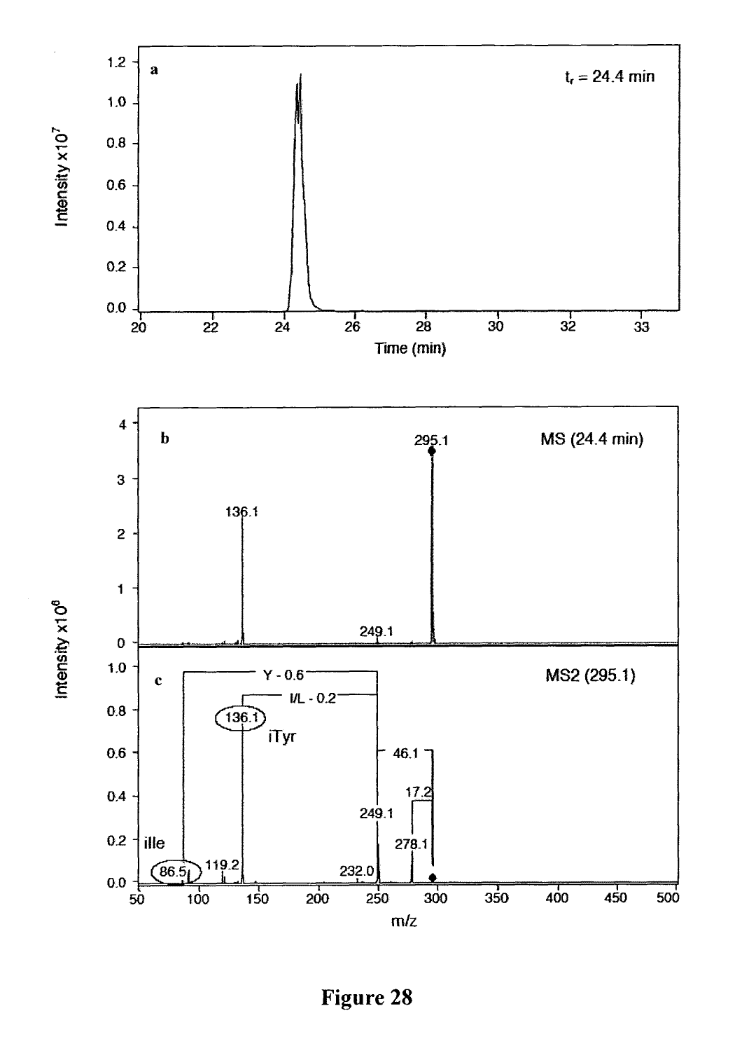

FIG. 28 illustrates the EIC and the MS and MS/MS spectra of the chemically-synthesized Tyr-Ile. An EIC peak is detected at 24.4 minutes (FIG. 28a).

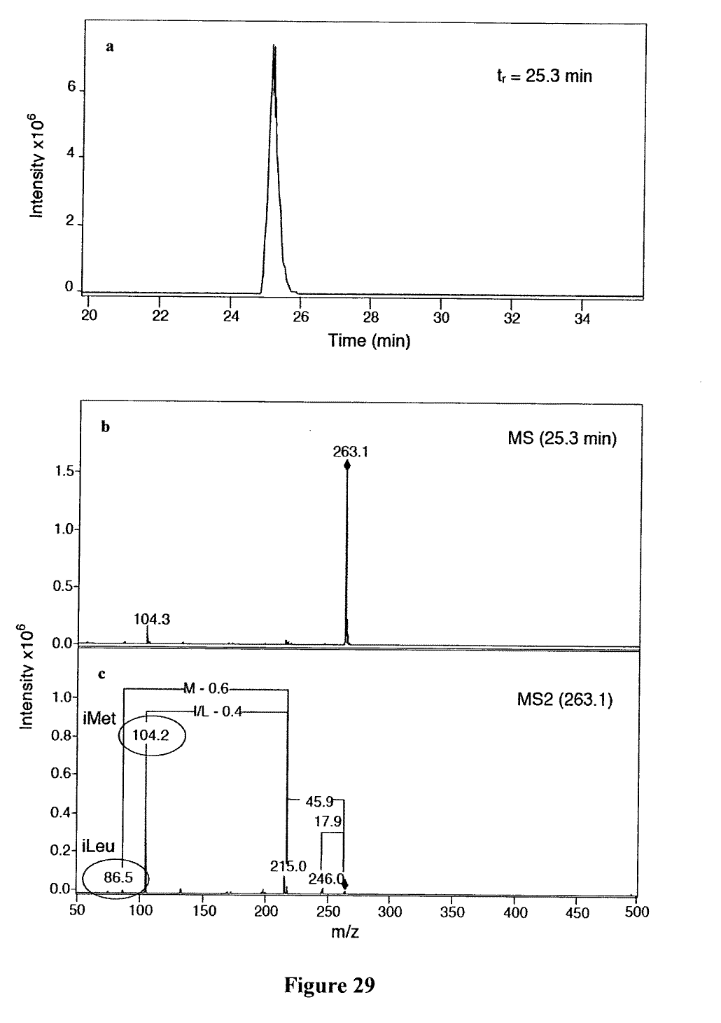

FIG. 29 illustrates the EIC and the MS and MS/MS spectra of the chemically-synthesized Met-Leu. An EIC peak is detected at 25.3 minutes (FIG. 29a).

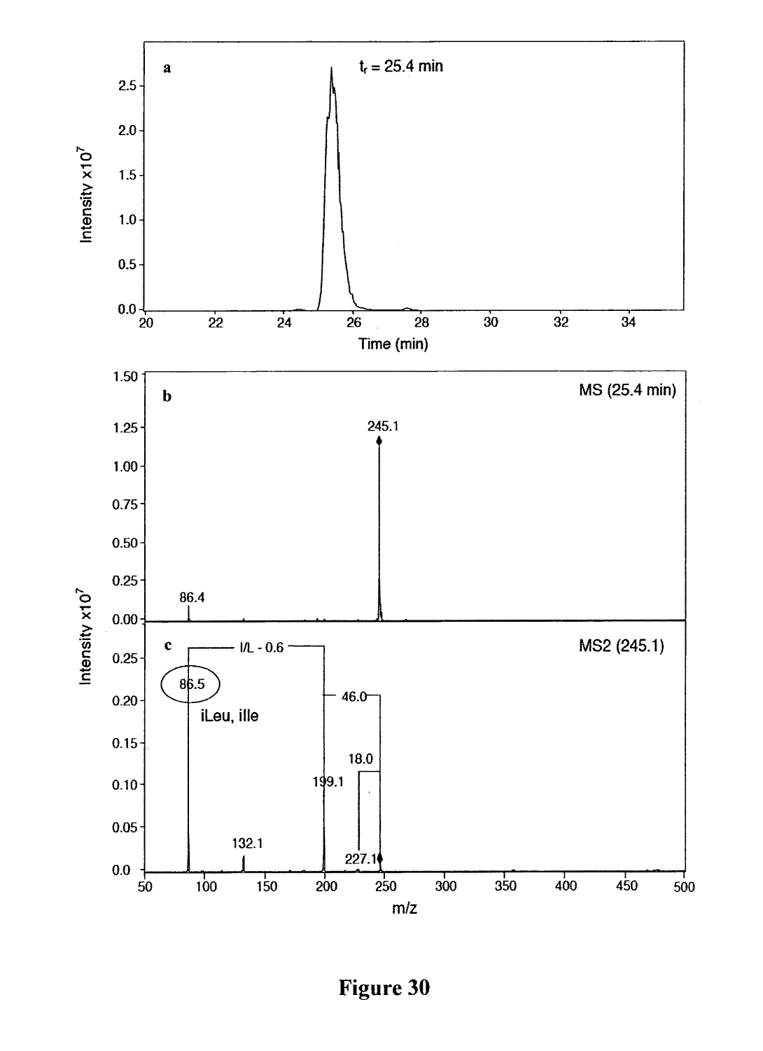

FIG. 30 illustrates the EIC and the MS and MS/MS spectra of the chemically-synthesized Leu-Ile. An EIC peak is detected at 25.4 minutes (FIG. 30a).

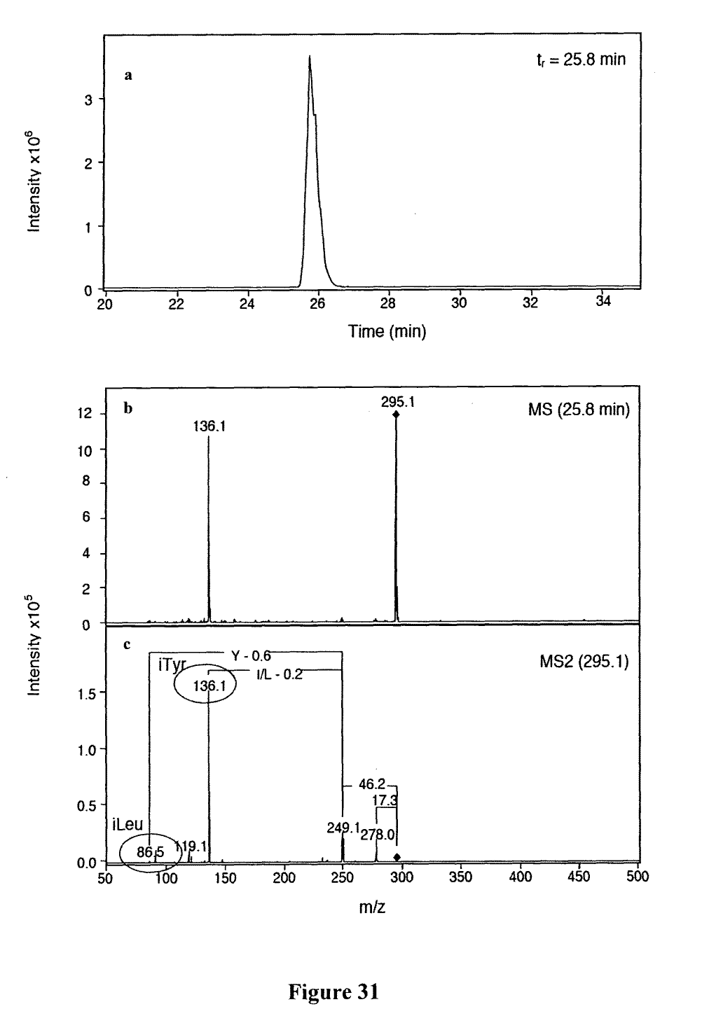

FIG. 31 illustrates the EIC and the MS and MS/MS spectra of the chemically-synthesized Tyr-Leu. An EIC peak is detected at 25.8 minutes (FIG. 31a).

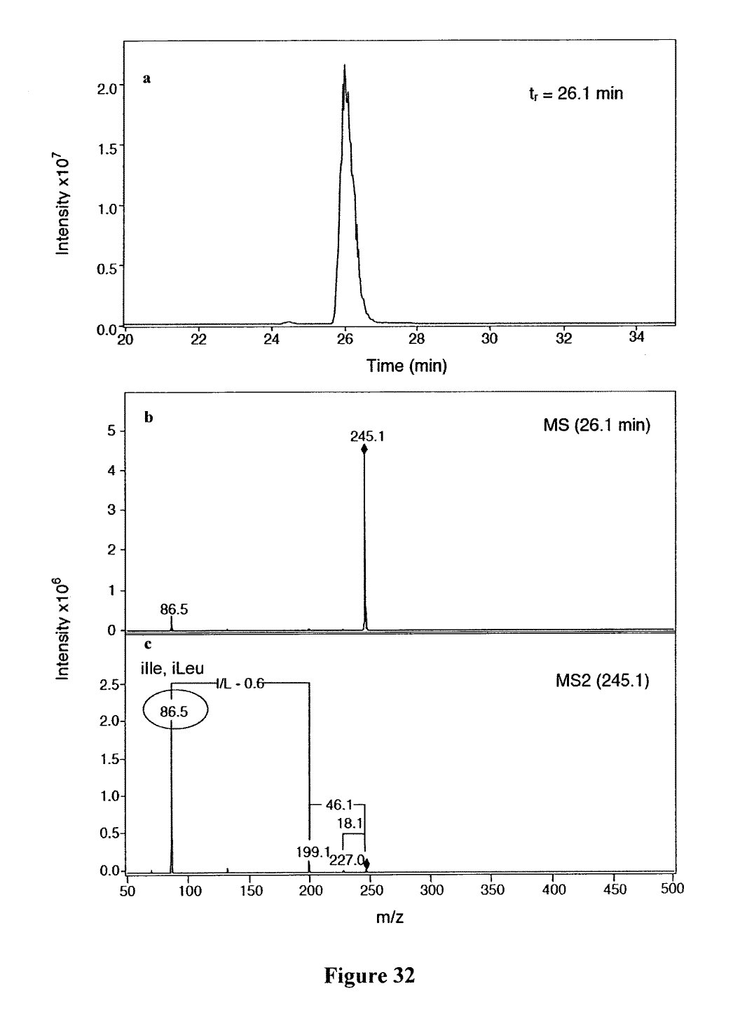

FIG. 32 illustrates the EIC and the MS and MS/MS spectra of the chemically-synthesized Ile-Leu. An EIC peak is detected at 26.1 minutes (FIG. 32a).

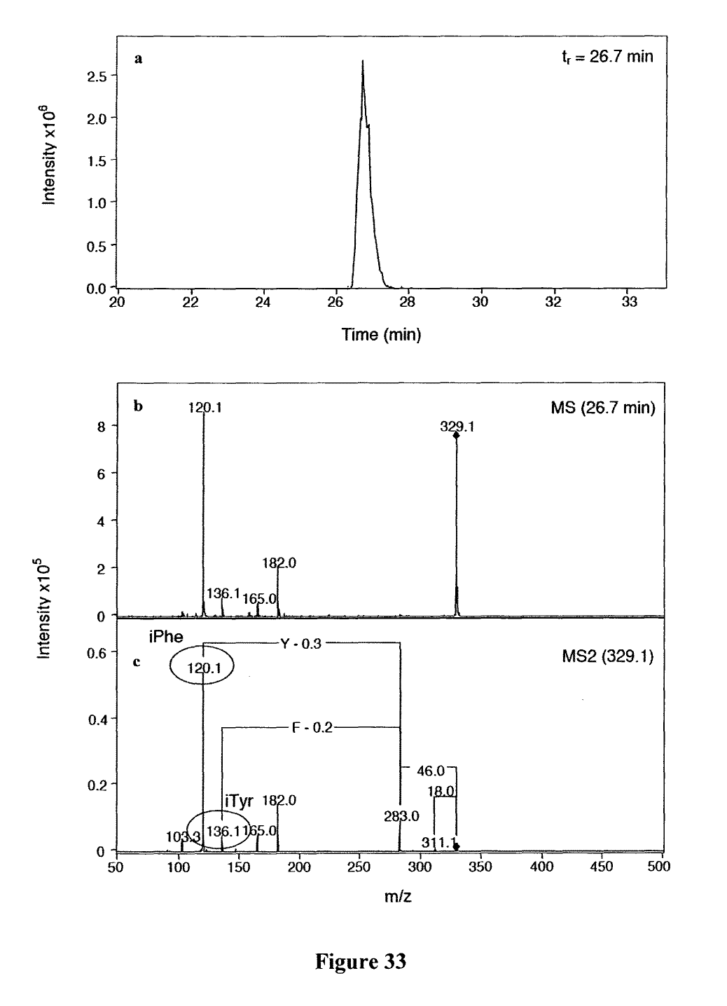

FIG. 33 illustrates the EIC and the MS and MS/MS spectra of the chemically-synthesized Phe-Tyr. An EIC peak is detected at 26.7 minutes (FIG. 33a).

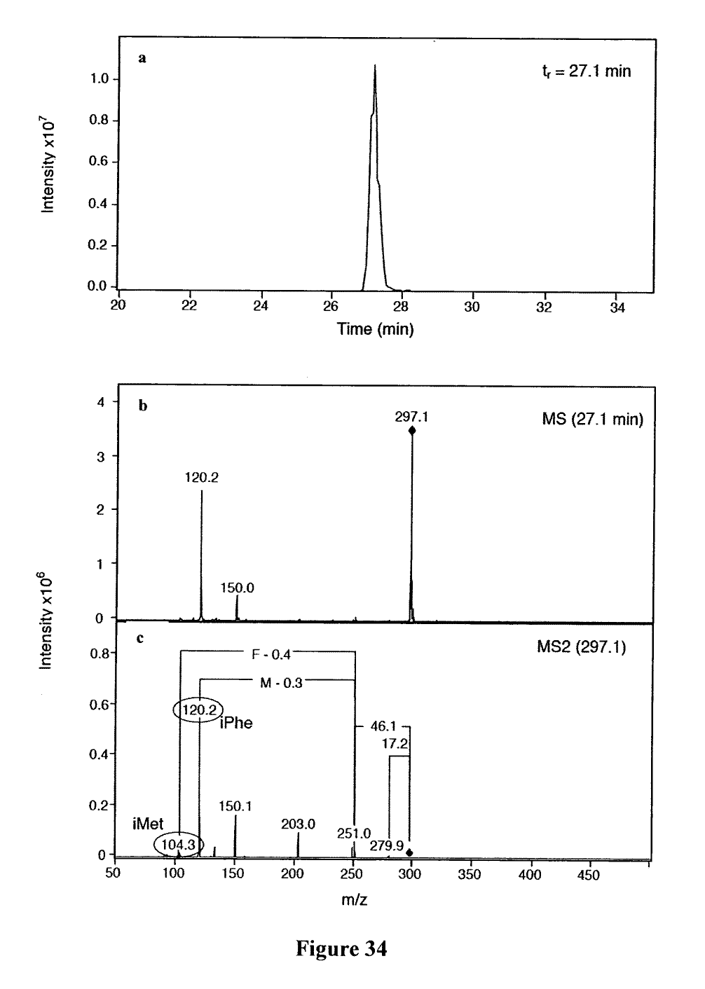

FIG. 34 illustrates the EIC and the MS and MS/MS spectra of the chemically-synthesized Phe-Met. An EIC peak is detected at 27.1 minutes (FIG. 34a).

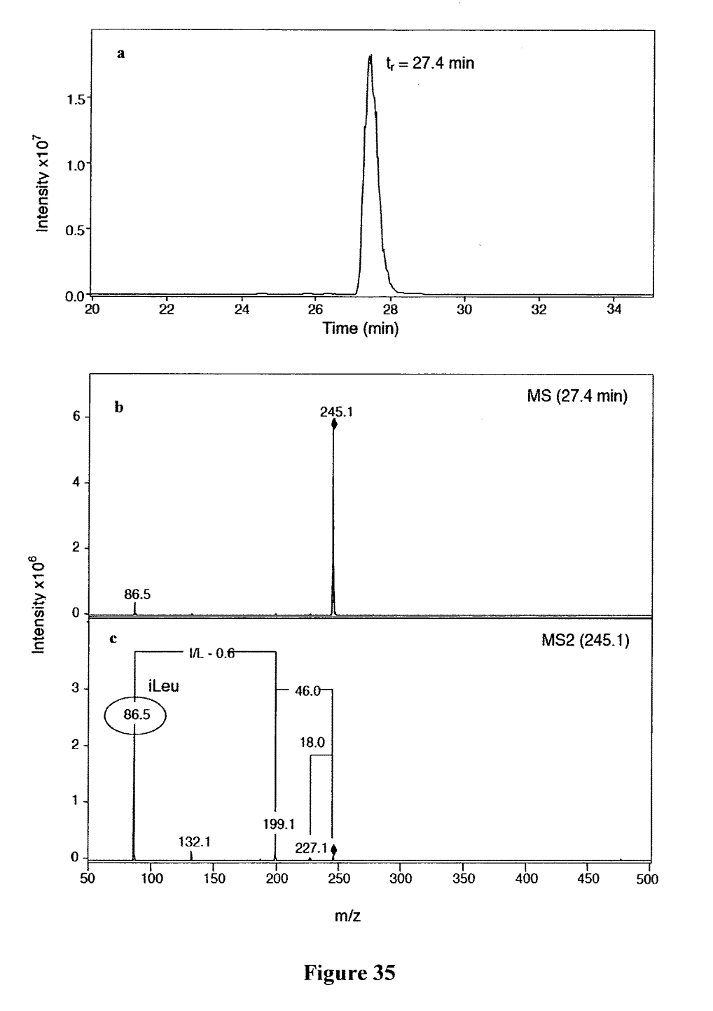

FIG. 35 illustrates the EIC and the MS and MS/MS spectra of the chemically-synthesized Leu-Leu. An EIC peak is detected at 27.4 minutes (FIG. 35a).

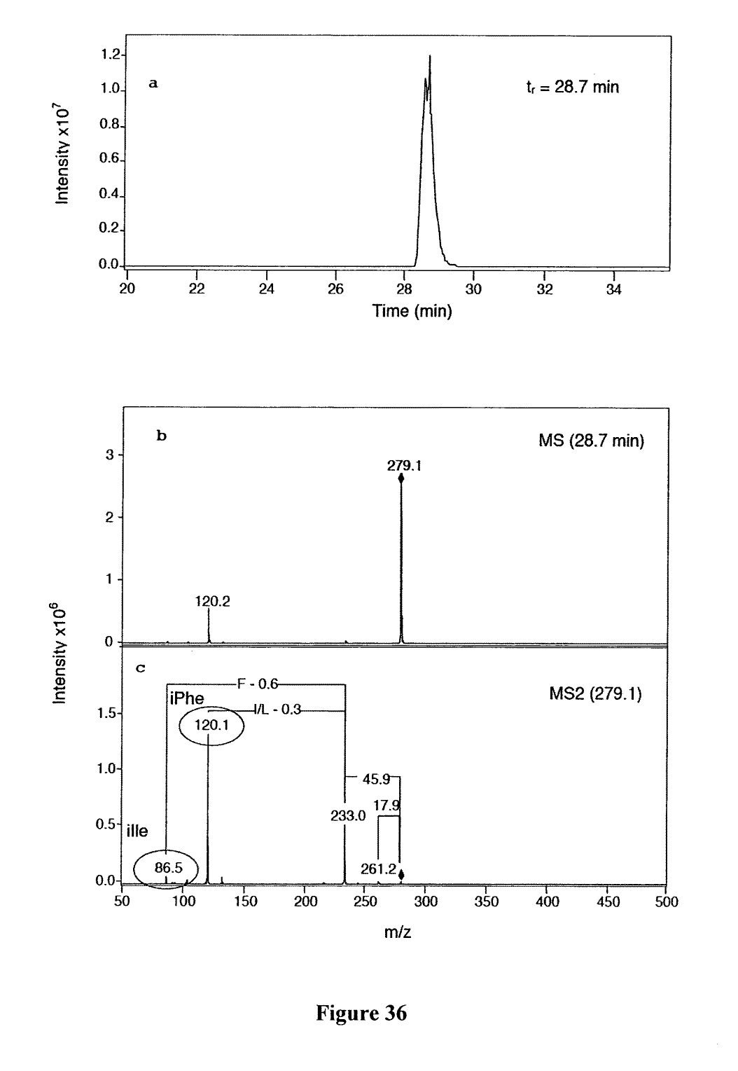

FIG. 36 illustrates the EIC and the MS and MS/MS spectra of the chemically-synthesized Phe-Ile. An EIC peak is detected at 28.7 minutes (FIG. 36a).

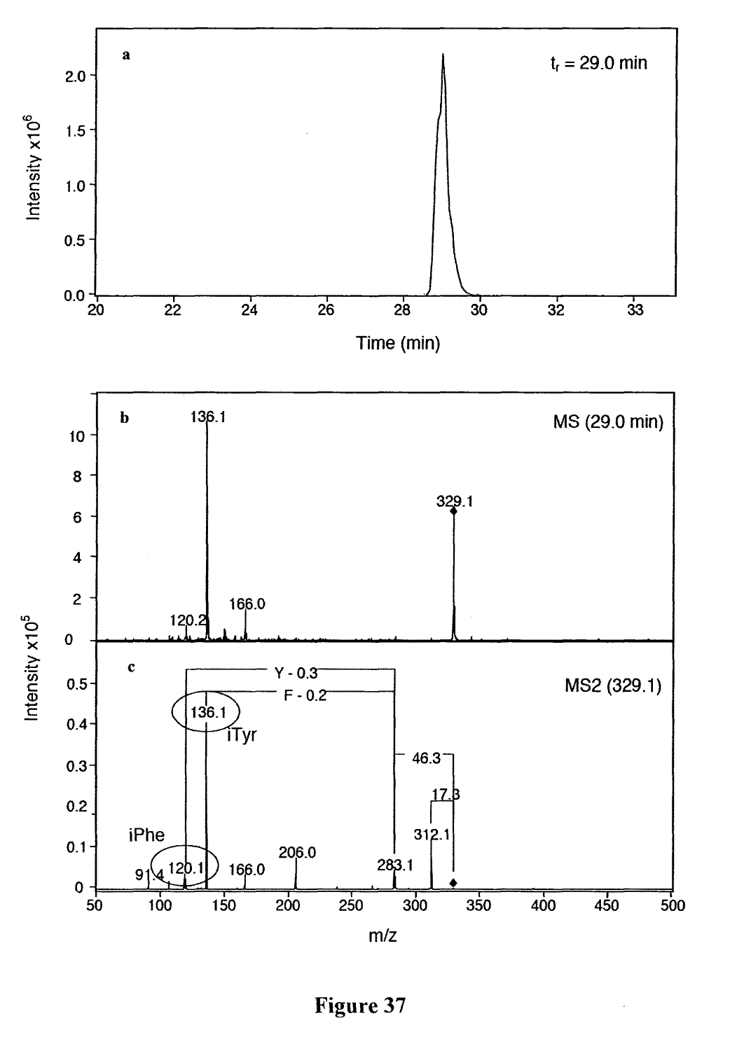

FIG. 37 illustrates the EIC and the MS and MS/MS spectra of the chemically-synthesized Tyr-Phe. An EIC peak is detected at 29.0 minutes (FIG. 37a).

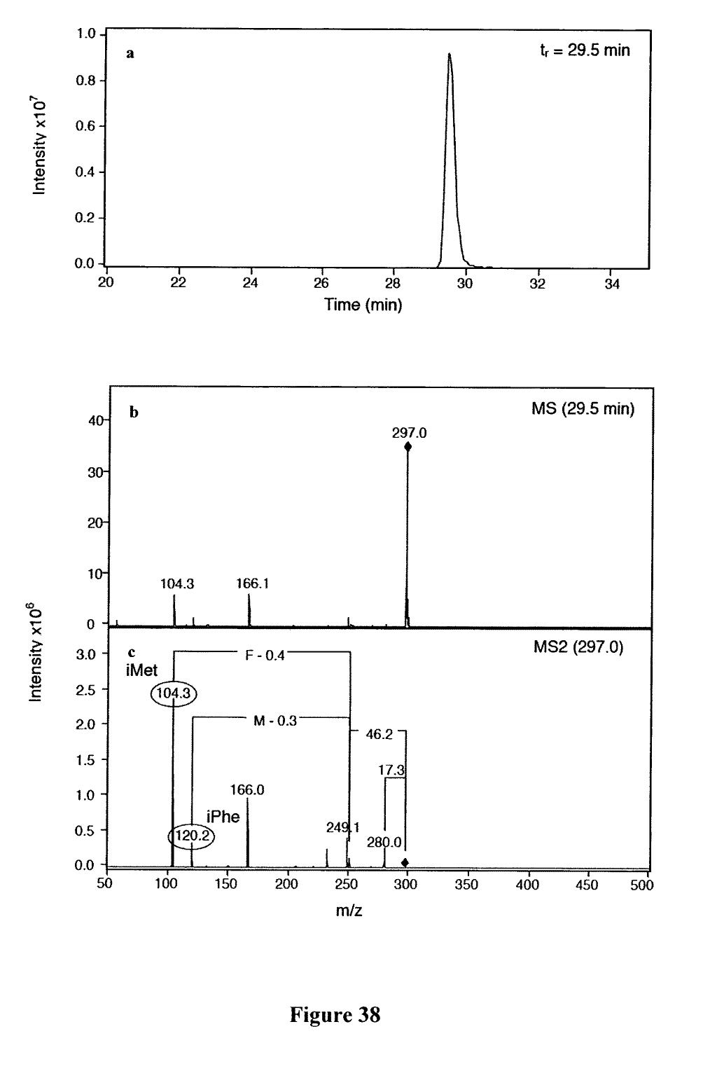

FIG. 38 illustrates the EIC and the MS and MS/MS spectra of the chemically-synthesized Met-Phe. An EIC peak is detected at 29.5 minutes (FIG. 38a).

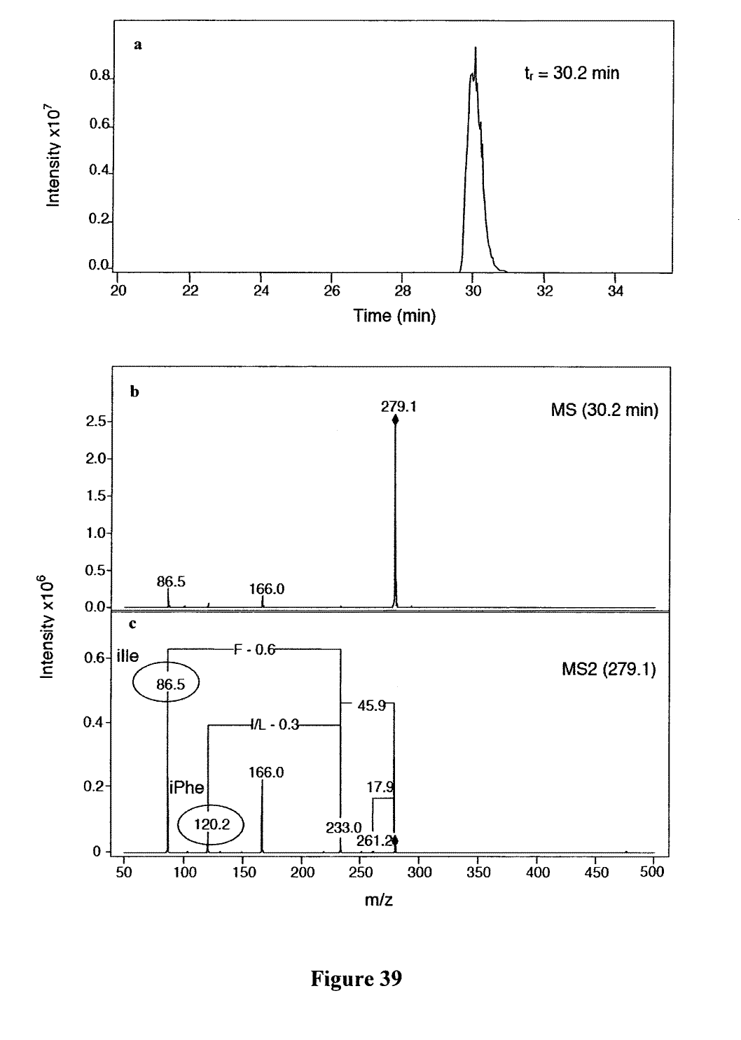

FIG. 39 illustrates the EIC and the MS and MS/MS spectra of the chemically-synthesized Ile-Phe. An EIC peak is detected at 30.2 minutes (FIG. 39a).

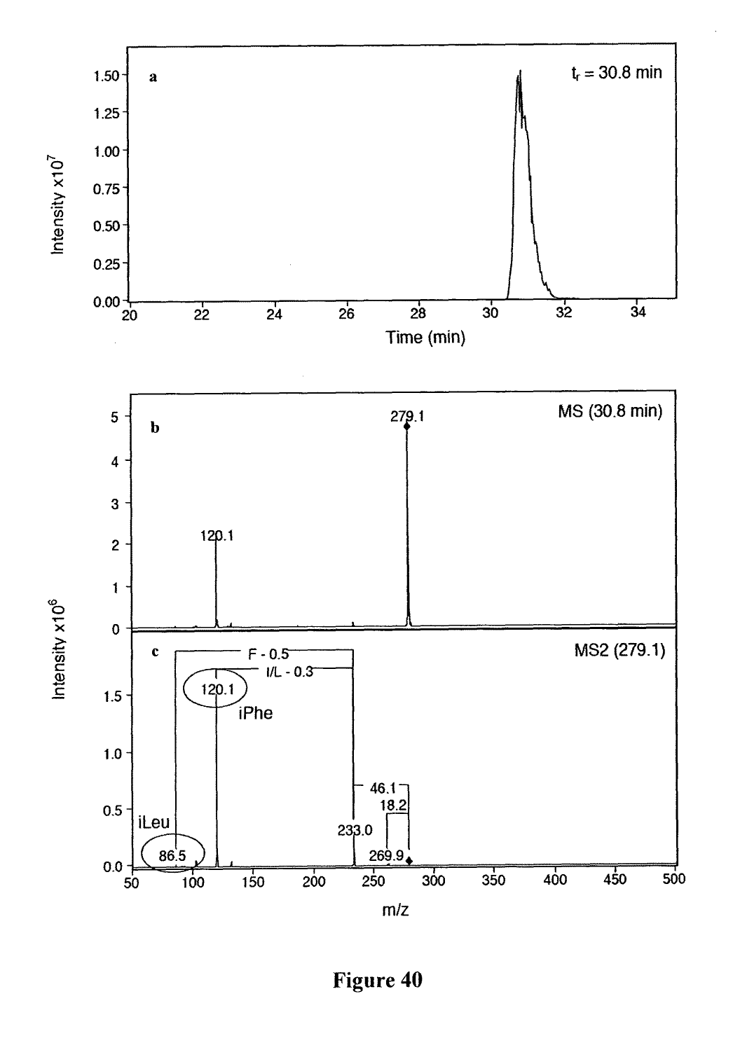

FIG. 40 illustrates the EIC and the MS and MS/MS spectra of the chemically-synthesized Phe-Leu. An EIC peak is detected at 30.8 minutes (FIG. 40a).

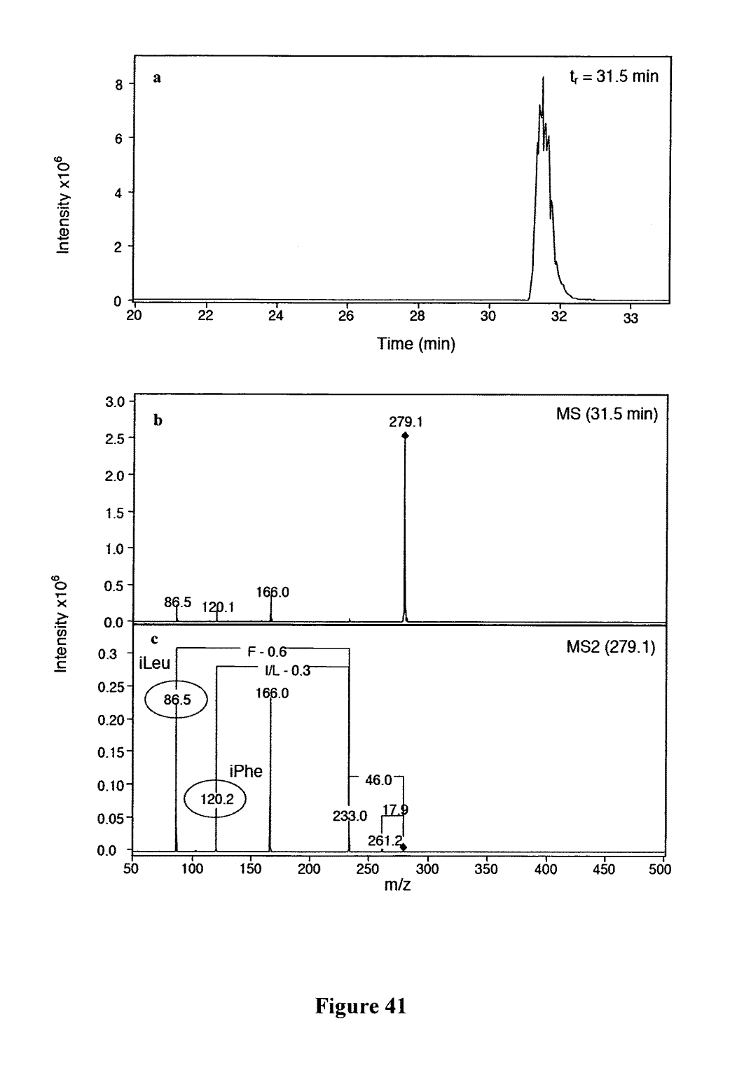

FIG. 41 illustrates the EIC and the MS and MS/MS spectra of the chemically-synthesized Leu-Phe. An EIC peak is detected at 31.5 minutes (FIG. 41a).

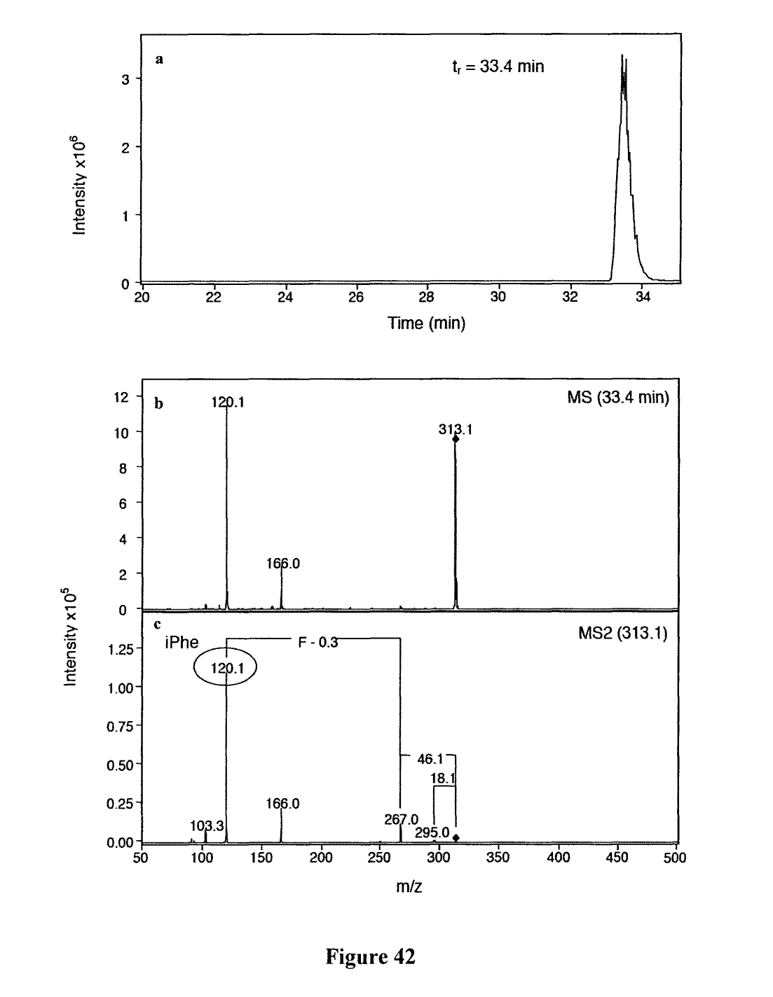

FIG. 42 illustrates the EIC and the MS and MS/MS spectra of the chemically-synthesized Phe-Phe. An EIC peak is detected at 33.4 minutes (FIG. 42a).

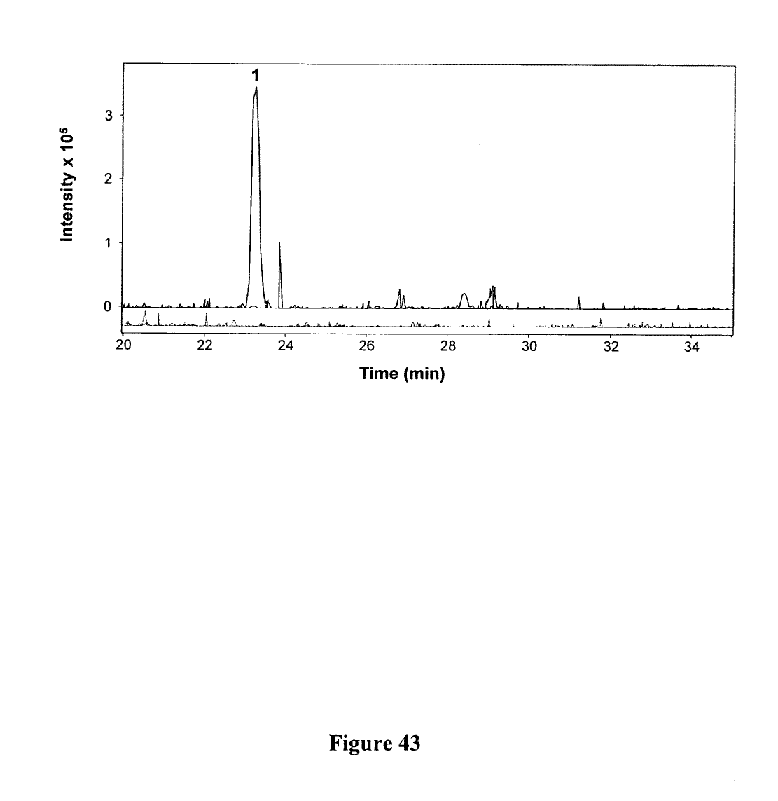

FIG. 43 illustrates EICs of dipeptides m/z values specific to Rv2275-his (SEQ ID NO:36) and detected from a LCMS analysis of the soluble fraction of E. coli cells expressing Rv2275-his (upper black traces) compared to the same set of EICs from a LCMS analysis of the control sample (lower grey traces).

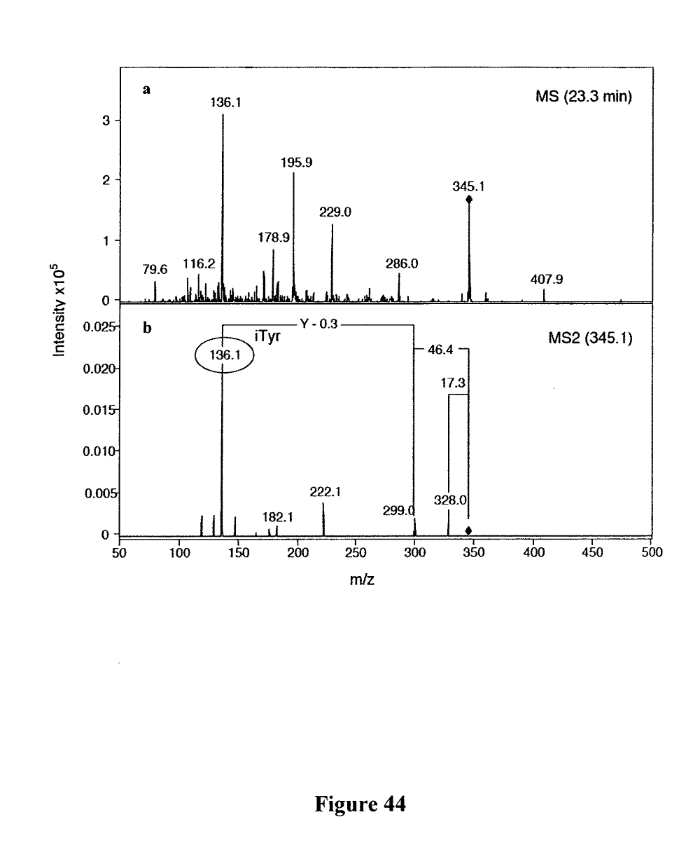

FIG. 44 illustrates the MS and MS/MS spectra of the EIC peak 1 detected at 23.3 min during the analysis of the soluble fraction of E. coli cells expressing Rv2275-his (SEQ ID NO:36).

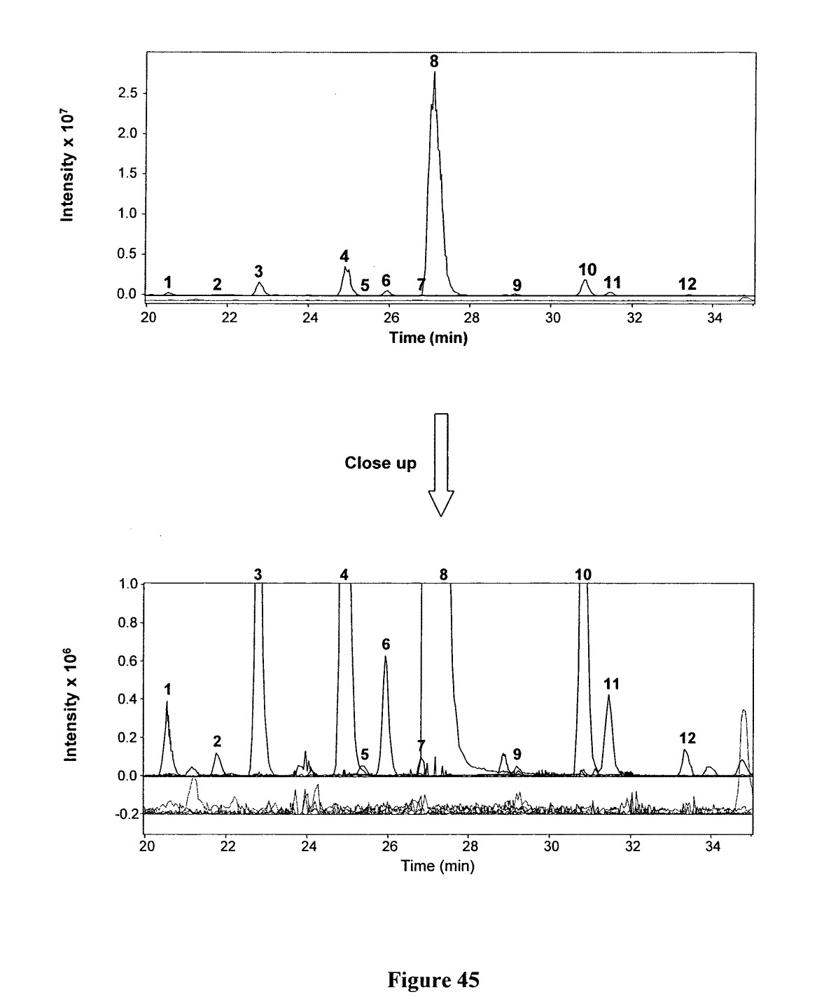

FIG. 45 illustrates EICs of dipeptides m/z values specific to YvmC-Bsub-his (SEQ ID NO:37) and detected from a LCMS analysis of the soluble fraction of E. coli cells expressing YvmC-Bsub-his (SEQ ID NO:37) (upper black traces) compared to the same set of EICs from a LCMS analysis of the control sample (lower grey traces).

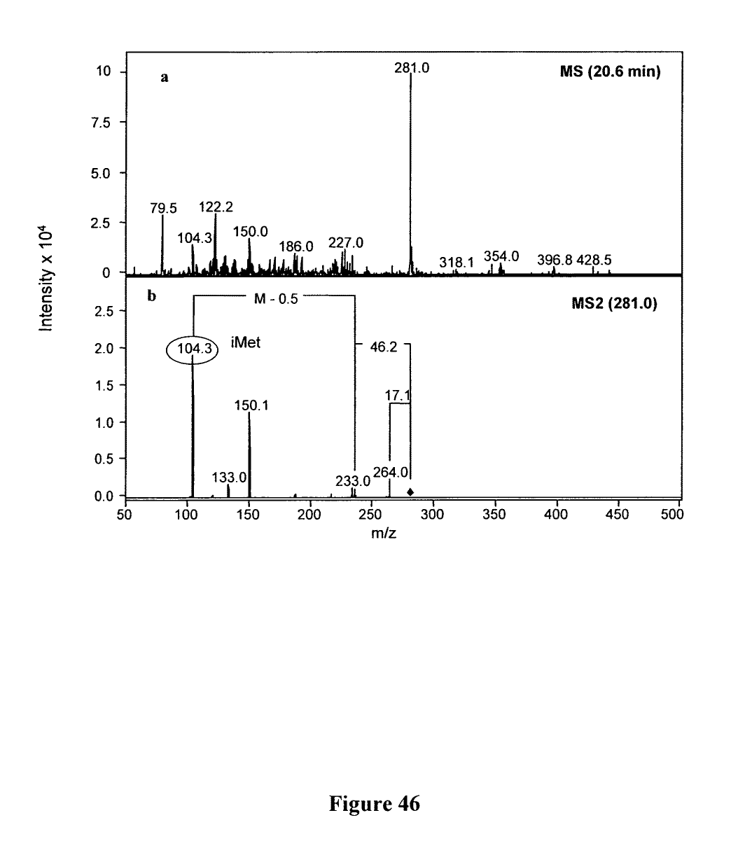

FIG. 46 illustrates the MS and MS/MS spectra of the EIC peak 1 detected at 20.6 min during the analysis of the soluble fraction of E. coli cells expressing YvmC.

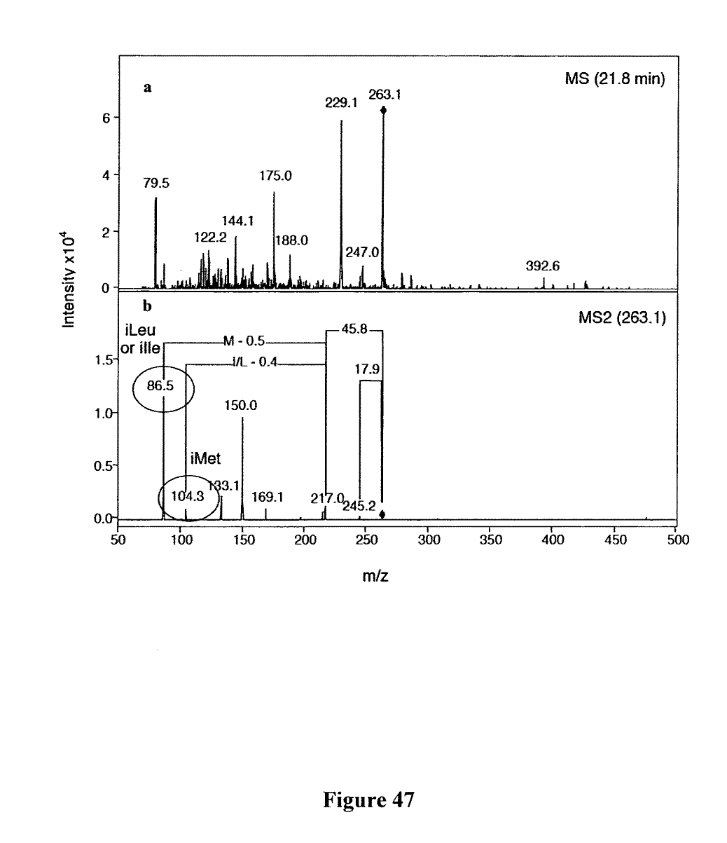

FIG. 47 illustrates the MS and MS/MS spectra of the EIC peak 2 detected at 21.8 min during the analysis of the soluble fraction of E. coli cells expressing YvmC.

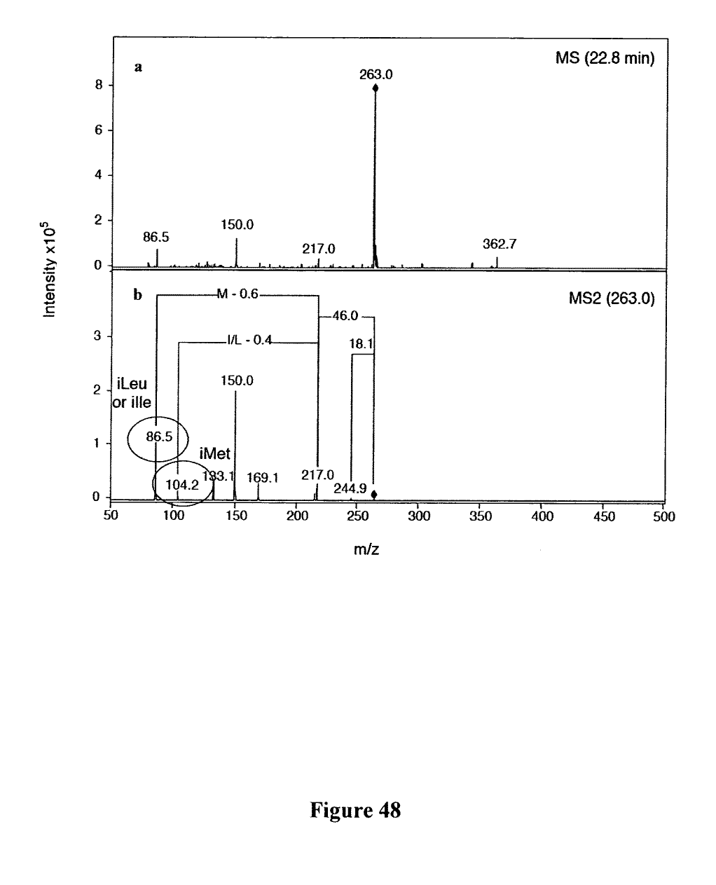

FIG. 48 illustrates the MS and MS/MS spectra of the EIC peak 3 detected at 22.8 min during the analysis of the soluble fraction of E. coli cells expressing YvmC.

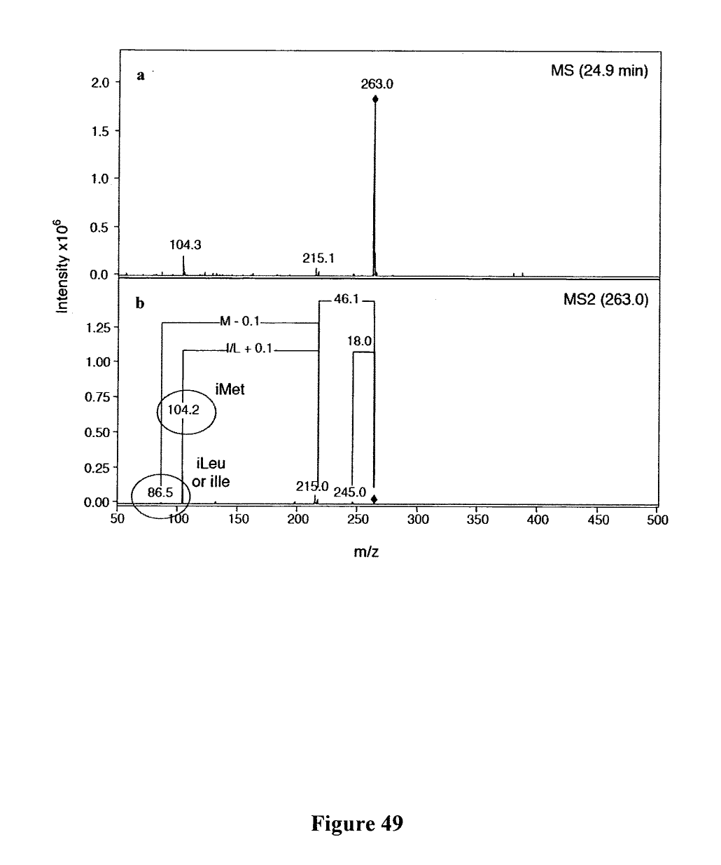

FIG. 49 illustrates the MS and MS/MS spectra of the EIC peak 4 detected at 24.9 min during the analysis of the soluble fraction of E. coli cells expressing YvmC.

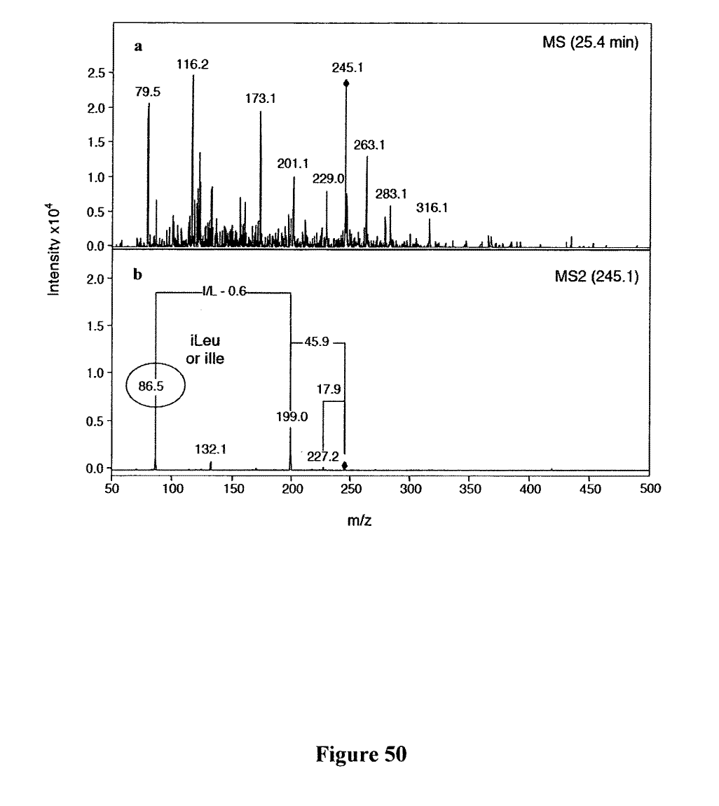

FIG. 50 illustrates the MS and MS/MS spectra of the EIC peak 5 detected at 25.4 min during the analysis of the soluble fraction of E. coli cells expressing YvmC.

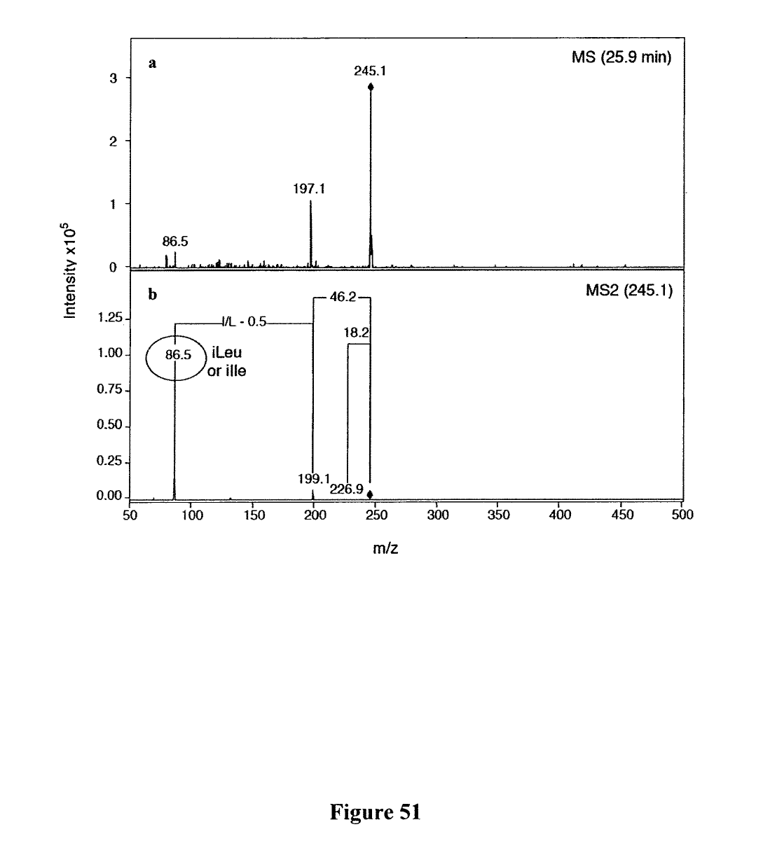

FIG. 51 illustrates the MS and MS/MS spectra of the EIC peak 6 detected at 25.9 min during the analysis of the soluble fraction of E. coli cells expressing YvmC.

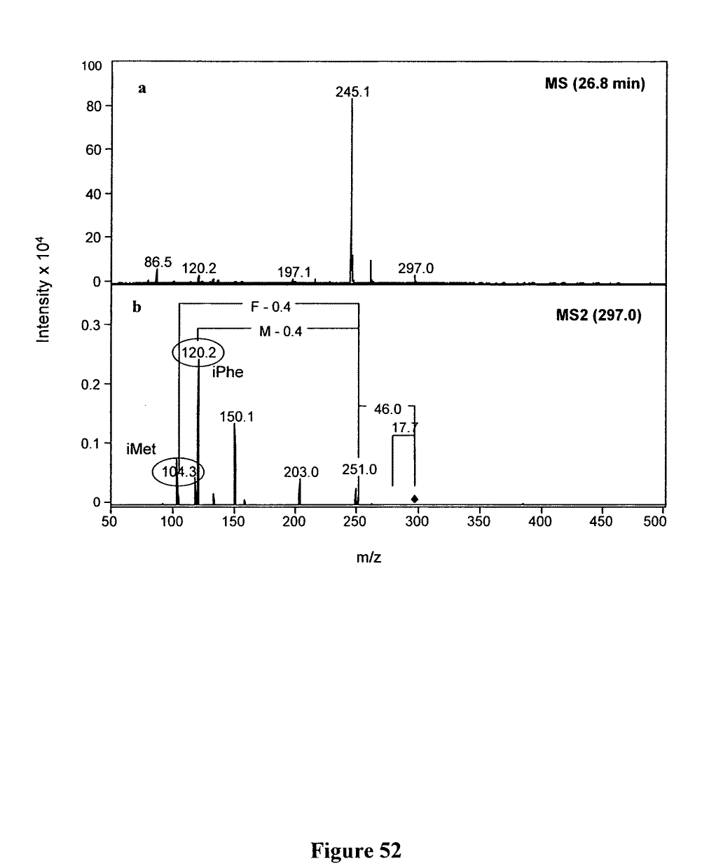

FIG. 52 illustrates the MS and MS/MS spectra of the EIC peak 7 detected at 26.8 min during the analysis of the soluble fraction of E. coli cells expressing YvmC.

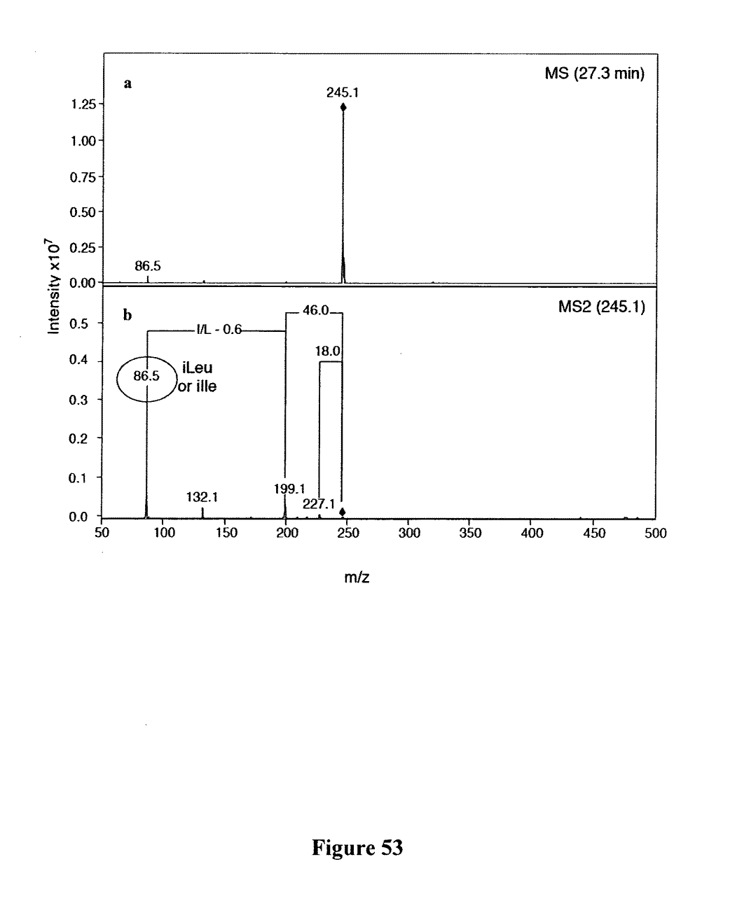

FIG. 53 illustrates the MS and MS/MS spectra of the EIC peak 8 detected at 27.3 min during the analysis of the soluble fraction of E. coli cells expressing YvmC.

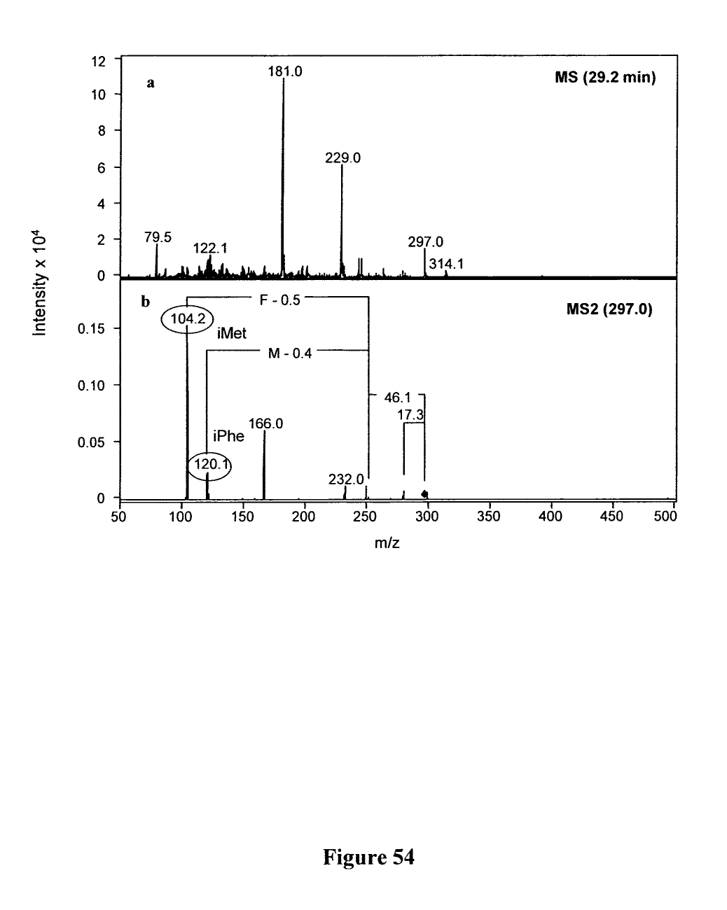

FIG. 54 illustrates the MS and MS/MS spectra of the EIC peak 9 detected at 29.2 min during the analysis of the soluble fraction of E. coli cells expressing YvmC.

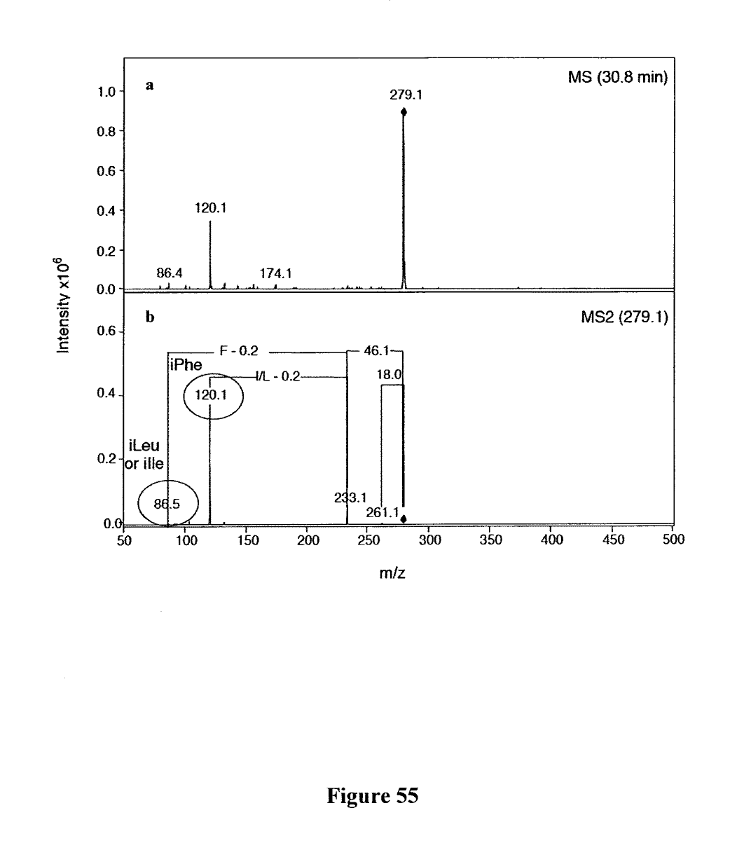

FIG. 55 illustrates the MS and MS/MS spectra of the EIC peak 10 detected at 30.8 min during the analysis of the soluble fraction of E. coli cells expressing YvmC.

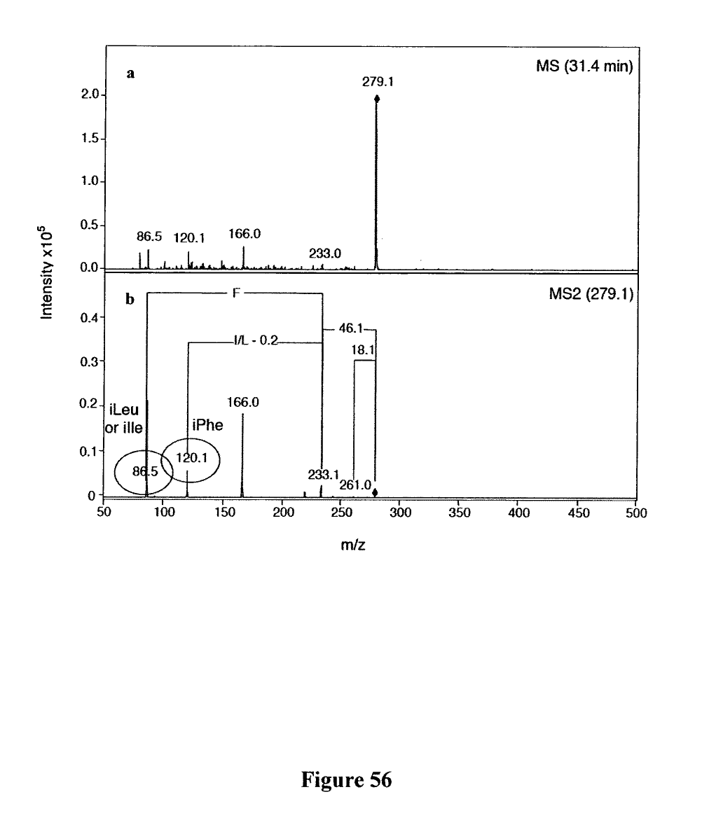

FIG. 56 illustrates the MS and MS/MS spectra of the EIC peak 11 detected at 31.4 min during the analysis of the soluble fraction of E. coli cells expressing YvmC.

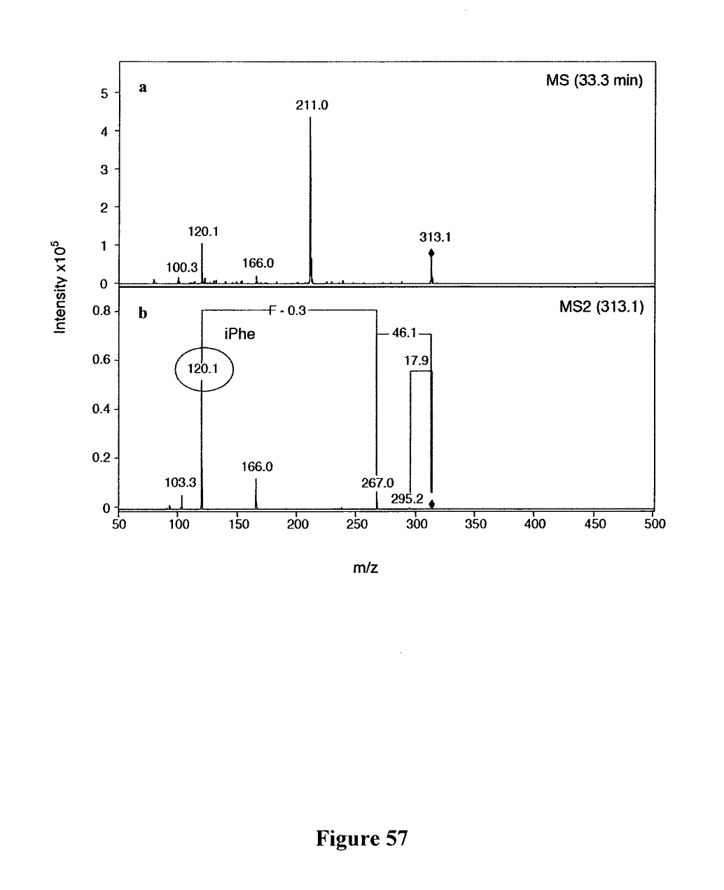

FIG. 57 illustrates the MS and MS/MS spectra of the EIC peak 12 detected at 33.3 min during the analysis of the soluble fraction of E. coli cells expressing YvmC.

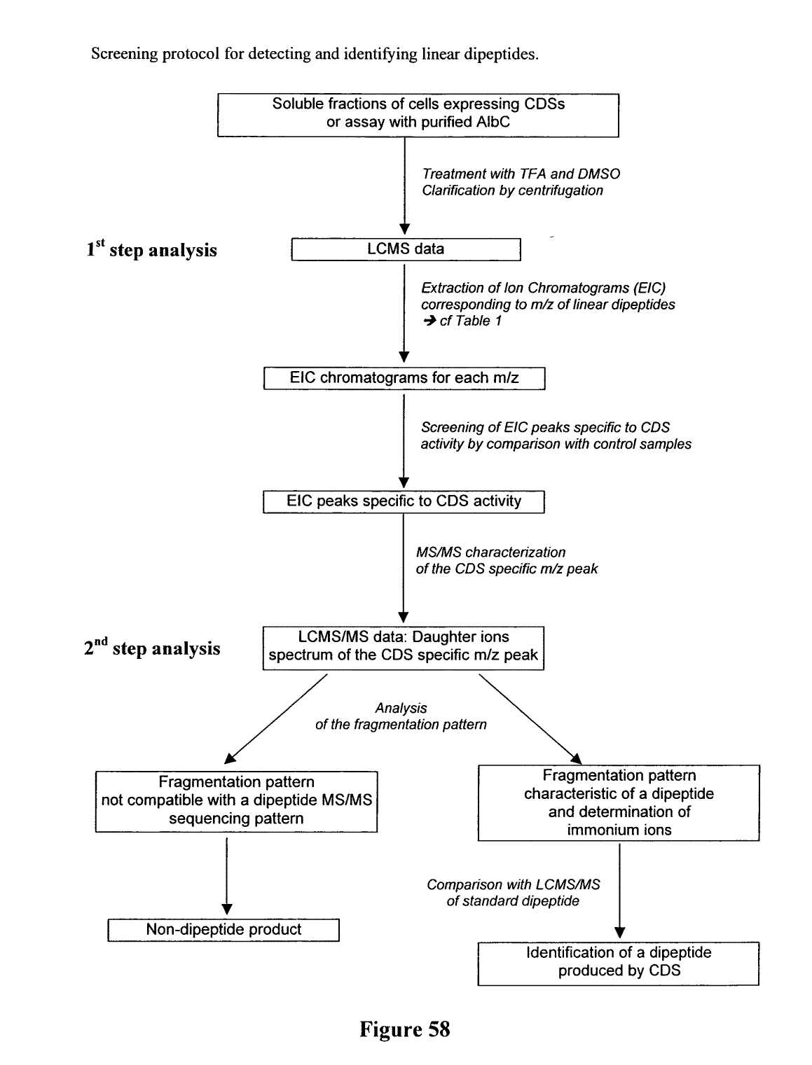

FIG. 58 summarizes an exhaustive screening protocol of linear dipeptides.

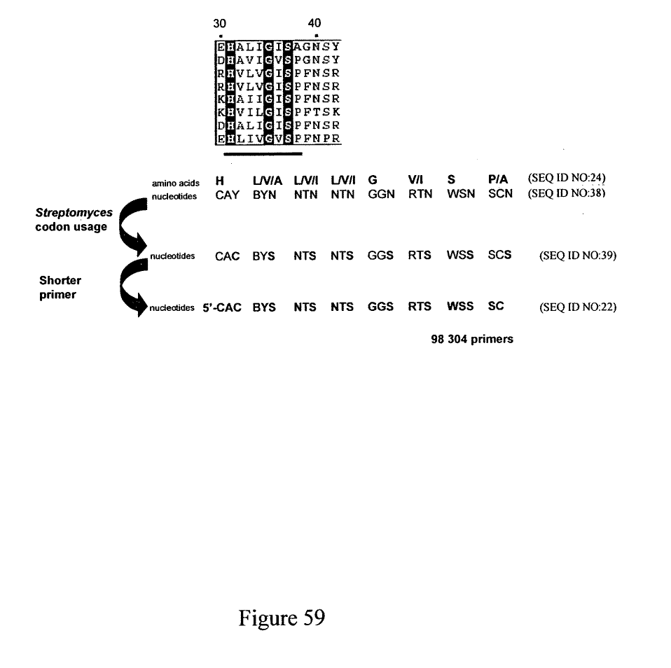

FIG. 59 shows a part of the alignment of all CDSs sequence and the region used for design of the first primer is indicated by a line under the alignment. The numbering is that of AlbC from S. noursei. The degenerated amino acid sequence is shown with the corresponding nucleotide sequence. For nucleotide: B=C or G or T, N=A or C or G or T, R=A or G, S=C or G, W=A or T, Y=C or T.

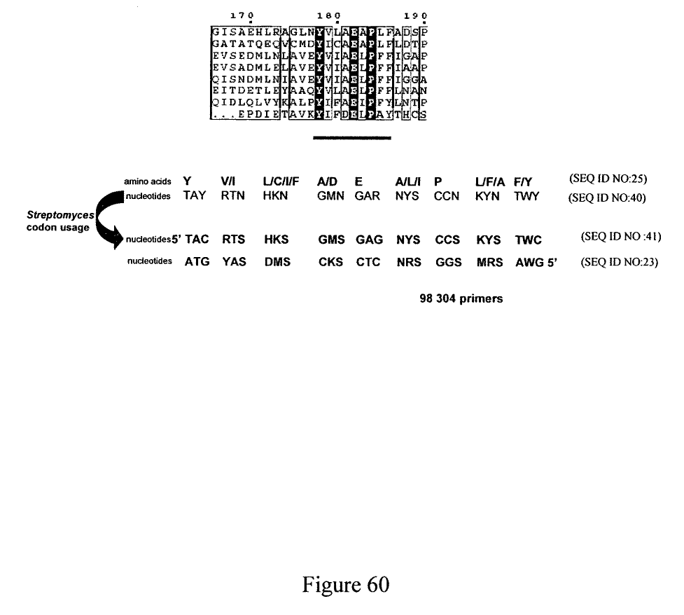

FIG. 60 shows a part of the alignment of all CDSs sequence and the region used for design of the second primer is indicated by a line under the alignment. The numbering is that of AlbC from S. noursei. The degenerated amino acid sequence is shown with the corresponding nucleotide sequence, and the complementary strand (at the bottom) used as primer. For nucleotide: D=A or G or T, K=G or T, M=A or C, N=A or C or G or T, R=A or G, S=C or G, W=A or T, Y=C or T.

There will now be described by way of example a specific mode contemplated by the Inventors. In the following description numerous specific details are set forth in order to provide a thorough understanding. It will be apparent however, to one skilled in the art, that the present invention may be practiced without limitation to these specific details. In other instances, well known methods and structures have not been described so as not to unnecessarily obscure the description.

### EXAMPLE 1

### Experimental Methods

1) Bioinformatic Tools.

The Basic Local Alignment Search Tool (BLAST) using the program default parameters to search for protein homologues (National Center for Biotechnology Information web site; http://www.ncb.nlm.nih.gov/BLAST/). Sequence alignments were performed using Multalin (Corpet, Nucleic Acids Res., 1988, 16, 10881-10890) (http://prodes.toulouse.inra.fr/multalin/multalin.html) or Clustal W (Thompson J D, Higgins D G, Gibson T J. Nucleic Acids Res. 1994, 22: 4673-4680 European Bioinformatics Institute web site; http://www.ebi.ac.uk/clustalw/index.html) with default parameters.

2) Construction of Escherichia coli Expression Vectors Encoding CDSs as C-terminal (His)-6-Tagged Fusions.

The sequences coding for AlbC, Rv2275 and YvmC-Bsub have been cloned into the E. coli expression vector pQE60 (Qiagen). For this, the coding sequences have been amplified by PCR (25 cycles using standard conditions) with primers designed to add a NcoI site overlapping the initiation codon and to add a BgIll site at the other end, following immediately the last sense codon. The PCR products were first cloned into the vector pGEMT-Easy vector (Promega) and then the NcoI-BglII fragment containing the coding sequence was cloned into pQE60 digested by NcoI and BglII. From the resulting pQE-60 derived plasmid, the protein is expressed with a 6×His C-terminal extension.

For AlbC, the primers used were 5′-AGAGCCATGGGACTTGCAGGCTTAGTTCCCGC-3′ SEQ ID NO:28 (NcoI site underlined) and 5′-AGAGAGATCTGGCCGCGTCGGCCAGCTCC-3′ SEQ ID NO:29 (BglII site underlined), the template was pSL122 (French Patent FR0207728, PCT/FR03/01851). The pQE60 derivative for AlbC expression was called pQE60-AlbC (SEQ ID NO:17); the expressed protein AlbC-his having the peptide sequence of SEQ ID NO:35.

For Rv2275, the primers used were 5′-CGGCCATGGCATACGTGGCTGCCGAACCAGGC-3′ SEQ ID NO:30 (NcoI site underlined) and 5′-GGCAGATCTTTCGGCGGGGCTCCCATCAGG-3′ SEQ ID NO:31 (BglII site underlined), the template was pEXP-Rv2275 (PCT/IB2006/001852). The pQE60 derivative for Rv2275 expression was called pQE60—Rv2275 (SEQ ID NO:18); the expressed protein Rv2275-his having the peptide sequence of SEQ ID NO:36.

For YvmC-Bsub from Bacillus subtilis, the primers used were 5′-GGCCCATGGCCGGAATGGTAACGGAAAGAAGGTCTG-3′ SEQ ID NO:32 (NcoI site underlined) and 5′-GGCAGATCTTCCTTCAGATGTGATCCGTTTCTCAGAAAGC-3′ SEQ ID NO:33 (BglII site underlined), the template was pEXP-YvmC-Bsub (PCT/IB2006/001849). The pQE60 derivative for YvmC-Bsub expression was called pQE60-YvmC-Bsub (SEQ ID NO:19); the expressed protein YvmC-Bsub-his having the peptide sequence of SEQ ID NO:37.

In all the above cases the native AlbC (SEQ ID NO:1), Rv2275 (SEQ ID NO:2) and YvmC-Bsub (SEQ ID NO:3) enzymes are functionally indistinguishable from the 6×His tag versions of these proteins AlbC-his (SEQ ID NO:35), Rv2275-his (SEQ ID NO:36) and YvmC-Bsub-his (SEQ ID NO:37) respectively expressed in the course of the experiments described herein. This is due to the fact that neither the modified second residue nor 6×His tag affect the functionality of either conserved portion of these enzymes. Also these modifications are not located close to or within these two conserved domains.

3) Assay for the In Vivo Formation of Linear Dipeptides by AlbC, Rv2275 and YvmC.

Recombinant expression of AlbC (SEQ ID NO:1) from S. noursei, Rv2275 (SEQ ID NO:2) from M. tuberculosis and YvmC-Bsub (SEQ ID NO:3) from B. subtilis, respectively as SEQ ID NO:35, SEQ ID NO:36 and SEQ ID NO:37, was achieved in E. coli M15pREP4 cells (Invitrogen) with the plasmids pQE60-AlbC(SEQ ID NO:17), pQE60—Rv2275 (SEQ ID NO:18) and pQE60-YvmC-Bsub (SEQ ID NO:19) respectively. 100 μl of chemically competent cells were transformed with 40 ng plasmid using standard heat-shock procedure (Sambrook et al., Molecular Cloning: A Laboratory manual, 2001, New York). After 1 h outgrowth at 37° C. with shaking in SOC medium, the 300 μl-reaction mixture was added directly to 5 ml LB medium containing 100 μg/ml ampicillin. After overnight incubation at 37° C. with shaking, this starter culture was used to inoculate 200 ml LB medium containing 100 μg/ml ampicillin. Bacteria were grown at 37° C. until OD₆₀₀˜0.7 and 1 mM IPTG was added. Culture was continued at 20° C. for 18 h. The bacterial cells were harvested by centrifugation (30 min, 5,000 g at 4° C.) and suspended in 5 ml ice-cold 9% NaCl solution. The cells were again harvested by centrifugation (30 min, 5,000 g at 4° C.) and suspended in lysis buffer A (100 mM Tris-HCl pH 8.0, 150 mM NaCl, 5% glycerol). The volume of the added lysis buffer was adjusted to obtain a bacterial suspension with an OD₆₀₀˜100. The suspended cells were then lysed with an Eaton press (Rassant). 5% dimethylsulfoxide (DMSO) was added to the lysate just before its centrifugation (30 min, 20,000 g at 4° C.). The soluble fraction was saved, acidified with 2% TFA and centrifuged (30 min, 20,000 g at 4° C.). The resulting soluble fraction was saved for further analysis by LC-MS/MS (see below).

As a control experiment, the whole process (from cell transformation to analysis of the linear dipeptide content) was applied to bacteria transformed by pQE60 (Qiagen), an ampicillin resistance gene-carrying vector that does not express CDS.

4. Samples Analysis by Chromatography Coupled On-Line to Mass Spectrometry.

Liquid Chromatography (LC) separation was carried out on a C18 analytical column (4.6×150 mm, 3 μm, 100 Å, Atlantis, Waters) at a flow rate of 600 μl/min with a 50 min linear gradient from 0 to 45% acetonitrile/MilliQ water with 0.1% formic acid after a 5 min step in the initial condition for column equilibration and sample desalting. Elution from the LC column was split into two flows: one at 550 μl/min directed to a diode array detector and the remaining flow directed to electrospray mass spectrometer for MS and MS/MS analyses. The mass spectrometer is an ion trap mass spectrometer Esquire HCT equipped with an orthogonal Atmospheric Pressure Interface-ElectroSpray Ionization (AP-ESI) source (Bruker Daltonik GmbH, Germany).

In this online coupling system, LC-eluted sample was continuously infused into the ESI probe at a flow rate of 50 μl/min. Nitrogen served as the drying and nebulizing gas while helium gas was introduced into the ion trap for efficient trapping and cooling of the ions generated by the ESI as well as for fragmentation processes. Ionization was carried out in positive mode with a nebulizing gas set at 35 psi, a drying gas set at 8 μl/min and a drying temperature set at 340° C. for optimal spray and desolvatation. Ionization and mass analyses conditions (capillary high voltage, skimmer and capillary exit voltages and ions transfer parameters) were tuned for an optimal detection of compounds over the range m/z 100 to 400. For structural characterization by mass fragmentations, an isolation width of 1 mass unit was used for isolating the parent ion. A fragmentation energy ramp was used for automatically varying the fragmentation amplitude in order to optimize the MS/MS fragmentation process. Full scan MS and MS/MS spectra were acquired using EsquireControl software and all data were processed using DataAnalysis software.

5) Chemical Synthesis of Linear Dipeptides.

Ile-Leu, Ile-Ile, Ile-Phe, Ile-Met, Phe-Ile, Leu-Met, Leu-Ile, Met-Ile and Tyr-Met were synthesized on an Applied Biosystems apparatus by conventional Fmoc/tBu strategy according to the user manual supplied with the apparatus (Applied Biosystems 433A User Manual Vol. 1, Chapter 3). Purification to homogeneity and physico-chemical characterization of linear peptides was achieved by RP-HPLC and mass spectrometry respectively. All other linear dipeptides were purchased from Sigma and Bachem.

6) Strategy Used for Detection and Identification of Linear Dipeptides.

The search for linear dipeptides was done according to an exhaustive screening protocol summarized in FIG. 58. All samples were analyzed by LC-MS/MS. From the LC-MS/MS data file, ion chromatograms corresponding to the 108 different m/z values associated with the 210 potential linear dipeptides (see Table I) were extracted. A set of extracted ion chromatograms (EICs) was then obtained for each CDS-containing samples as well as for control samples. For each m/z value, comparison of EICs obtained from CDS-containing sample and control sample enabled the detection of EIC peaks specific to CDS activity. These specific peaks were further characterized by MS/MS fragmentation for structural elucidation. Analysis of the daughter ions spectra enabled first to identify peaks corresponding to linear dipeptides. Indeed, linear dipeptides possess a specific fragmentation signature characterized by a combination of neutral losses of 17, 18, 28 and/or 46 (corresponding to fragmentations of the functional groups of peptides and fragmentations of the amide bond as previously proposed (Roepstorff et al., Biomed. Mass Spectrom., 1984, 11, 601; Johnson et al., Anal. Chem., 1987, 59, 2621-2625). Second, the analysis enabled to identify the two amino acids contained in the linear dipeptide either by the detection of immonium ions which are characteristic of amino acid side chains or by the neutral losses corresponding to the departure of amino acid residues constituting the linear dipeptide. The final identification of a linear dipeptide in a sample was obtained by confirming the similarity of both its retention time in LC and especially its fragmentation pattern in MS/MS with those of reference dipeptides (commercial or home-made synthetic dipeptides).

TABLE I

Calculated monoisotopic mass (m/z) values of
natural dipeptides under positive mode of ESI-MS.

| AA | Gly | Ala | Ser | Pro | Val | Thr | Cys | Ile | Leu | Asn |
|---|---|---|---|---|---|---|---|---|---|---|
| residue | 57.05 | 71.08 | 87.08 | 97.12 | 99.13 | 101.1 | 103.1 | 113.2 | 113.2 | 114.1 |
| Gly | 133.0 | 147.1 | 163.1 | 173.1 | 175.1 | 177.1 | 179.0 | 189.1 | 189.1 | 190.1 |
| Ala | | 161.1 | 177.1 | 187.1 | 189.1 | 191.1 | 193.0 | 203.1 | 203.1 | 204.1 |
| Ser | | | 193.1 | 203.1 | 205.1 | 207.1 | 209.0 | 219.1 | 219.1 | 220.1 |
| Pro | | | | 213.1 | 215.1 | 217.1 | 219.1 | 229.1 | 229.1 | 230.1 |
| Val | | | | | 217.1 | 219.1 | 221.1 | 231.2 | 231.2 | 232.1 |
| Thr | | | | | | 221.1 | 223.1 | 233.1 | 233.1 | 234.1 |
| Cys | | | | | | | 225.0 | 235.1 | 235.1 | 236.1 |
| Ile | | | | | | | | 245.2 | 245.2 | 246.1 |
| Leu | | | | | | | | | 245.2 | 246.1 |
| Asn | | | | | | | | | | 247.1 |
| Asp | | | | | | | | | | |
| Gln | | | | | | | | | | |
| Lys | | | | | | | | | | |
| Glu | | | | | | | | | | |
| Met | | | | | | | | | | |
| His | | | | | | | | | | |
| Phe | | | | | | | | | | |
| Arg | | | | | | | | | | |
| Tyr | | | | | | | | | | |
| Trp | | | | | | | | | | |
| AA | Asp | Gln | Lys | Glu | Met | His | Phe | Arg | Tyr | Trp |
| residue | 115.1 | 128.1 | 128.2 | 129.1 | 131.2 | 137.1 | 147.2 | 156.2 | 163.2 | 186.2 |
| Gly | 191.0 | 204.1 | 204.1 | 205.1 | 207.1 | 213.1 | 223.1 | 232.1 | 239.1 | 262.1 |
| Ala | 205.1 | 218.1 | 218.1 | 219.1 | 221.1 | 227.1 | 237.1 | 246.1 | 253.1 | 276.1 |
| Ser | 221.1 | 234.1 | 234.1 | 235.1 | 237.1 | 243.1 | 253.1 | 262.1 | 269.1 | 292.1 |
| Pro | 231.1 | 244.1 | 244.1 | 245.1 | 247.1 | 253.1 | 263.1 | 272.2 | 279.1 | 302.1 |
| Val | 233.1 | 246.1 | 246.2 | 247.1 | 249.1 | 255.1 | 265.1 | 274.2 | 281.1 | 304.1 |
| Thr | 235.1 | 248.1 | 248.1 | 249.1 | 251.1 | 257.1 | 267.1 | 276.1 | 283.1 | 306.1 |
| Cys | 237.0 | 250.1 | 250.1 | 251.1 | 253.0 | 259.1 | 269.1 | 278.1 | 285.1 | 308.1 |
| Ile | 247.1 | 260.1 | 260.2 | 261.1 | 263.1 | 269.1 | 279.2 | 288.2 | 295.1 | 318.2 |
| Leu | 247.1 | 260.1 | 260.2 | 261.1 | 263.1 | 269.1 | 279.2 | 288.2 | 295.1 | 318.2 |
| Asn | 248.1 | 261.1 | 261.1 | 262.1 | 264.1 | 270.1 | 280.1 | 289.1 | 296.1 | 319.1 |
| Asp | 249.1 | 262.1 | 262.1 | 263.1 | 265.1 | 271.1 | 281.1 | 290.1 | 297.1 | 320.1 |
| Gln | | 275.1 | 275.2 | 276.1 | 278.1 | 284.1 | 294.1 | 303.2 | 310.1 | 333.1 |
| Lys | | | 275.2 | 276.1 | 278.1 | 284.2 | 294.2 | 303.2 | 310.2 | 333.2 |
| Glu | | | | 277.1 | 279.1 | 285.1 | 295.1 | 304.1 | 311.1 | 334.1 |
| Met | | | | | 281.1 | 287.1 | 297.1 | 306.1 | 313.1 | 336.1 |
| His | | | | | | 293.1 | 303.1 | 312.2 | 319.1 | 342.1 |
| Phe | | | | | | | 313.1 | 322.2 | 329.1 | 352. [symbol] |
| Arg | | | | | | | | 331.2 | 338.2 | 361. [symbol] |
| Tyr | | | | | | | | | 345.1 | 368. [symbol] |
| Trp | | | | | | | | | | 391. [symbol] |

[symbol]

indicates data missing or illegible when filed

### EXAMPLE 2

### The In Vivo Synthesis of Linear Dipeptides by CDSs

Synthesis of linear dipeptides by CDSs was assessed by searching for linear dipeptides in soluble extracts obtained from bacteria expressing respectively AlbC, Rv2275 and YvmC-Bsub, in each case these enzymes were expressed with a C-terminal 6-his tag, also the second residue was modified due the introduction of the NcoI restriction enzyme target sequence into these sequences to allow cloning into the pQE60 vector as previously described (see Experimental Methods). The actual peptide sequence of each enzyme expressed being AlbC-his SEQ ID NO:35, Rv2275-his SEQ ID NO:36 and YvmC-Bsub-his SEQ ID NO:37. These extracts were performed as previously described (see Experimental Methods) and, in each case, the production of a protein whose molecular weight and N-terminal sequence corresponded to those expected was observed. At the same time, a soluble extract obtained from bacteria expressing no CDS (pQE60) was also prepared. Finally, all these samples were analyzed by LC-MS/MS and screened for linear dipeptides as depicted in FIG. 58. As a method control, the soluble fraction of E. coli cells expressing AlbC-his (SEQ ID NO:35) was first analyzed.

1) Additional Linear Dipeptides Produced in the Presence of AlbC.

The soluble fraction of E. coli cells expressing AlbC-his (SEQ ID NO:35) was analyzed by LC-MS/MS leading to a first set of EICs. The same analysis was performed with the soluble fraction of E. coli cells not expressing AlbC-his (SEQ ID NO:35) leading to a second set of EICs. Comparison of the two sets of EICs for each m/z value enabled the detection of EIC peaks specific to the AlbC activity. Each EIC peak was characterized by MS/MS fragmentation and the analysis of the daughter ions spectra indicated that 15 peaks (shown in FIG. 2) matched with linear dipeptides (see summary shown as Table II).

The mass characteristics of each of the 15 EIC peaks, in particular the detection of immonium ions, led to the unambiguous identification of the amino acids constituting 8 different dipeptides corresponding to peak 1, peak 2, peak 3, peak 8, peak 9, peak 11, peak 12, and peak 15 (Table II). The nature of the amino acids constituting the other dipeptides, corresponding to peak 4, peak 5, peak 6, peak 7, peak 10, peak 13 and peak 14, remained to be confirmed because they all contain leucyl or isoleucyl residues (see Table II) that have identical immonium ion m/z of 86.5. The identification of the nature and also the sequence of all detected linear dipeptides was definitely achieved by comparing their retention times in LC and also their fragmentation patterns in MS/MS—i.e. number of fragments ions, m/z values, and intensities of the generated fragments ions—(see Table II and figures numbered herein) to those of reference chemically-synthesized dipeptides (see Table III and figures numbered herein). Due to LC column ageing, the retention times of 3 detected linear dipeptides were shifted compared to those of corresponding reference dipeptides—namely Met-Met, Tyr-Met and Met-Tyr—but the elution order was the same for detected and reference dipeptides. Taken together all these data established clearly that AlbC expression in E. coli cells is responsible for the in vivo formation of Leu-Phe and Phe-Leu as previously reported (U.S. Pat. U.S. N^{o} 20050287626) and also Phe-Phe, Phe-Tyr, Tyr-Phe, Leu-Leu, Leu-Tyr, Tyr-Leu, Phe-Met, Met-Phe, Leu-Met, Met-Leu, Met-Met, Tyr-Met and Met-Tyr (see Tables II & III).

TABLE II
LC-MS/MS analysis of the soluble fraction of E. coli cells
expressing AlbC: summary of data extracted from figures
whose numbers are reported herein and identification of
linear dipeptides.

| | MS and MS/MS data | | | | |
|---|---|---|---|---|---|
| | | | See | | |
| EIC | | Immonium | Figures | LC Data | Identified |
| Peaks^{a} | m/z | ions detected | (n^{o}) | Tr (min)^{b} | dipeptides^{c} |
| 1 | 281.0 | iMet | 3 | 20.6 | Met-Met |
| 2 | 313.1 | iTyr, iMet | 4 | 22.0 | Met-Tyr |
| 3 | 313.1 | iTyr, iMet | 5 | 22.5 | Tyr-Met |
| 4 | 263.0 | iMet, iLeu or iIle | 6 | 22.9 | Leu-Met |
| 5 | 295.1 | iTyr, iLeu or iIle | 7 | 23.8 | Leu-Tyr |
| 6 | 263.0 | iMet, iLeu or iIle | 8 | 25.0 | Met-Leu |
| 7 | 295.1 | iTyr, iLeu or iIle | 9 | 25.9 | Tyr-Leu |
| 8 | 329.1 | iPhe, iTyr | 10 | 26.6 | Phe-Tyr |
| 9 | 297.1 | iMet, iPhe | 11 | 27.0 | Phe-Met |
| 10 | 245.1 | iLeu or iIle | 12 | 27.3 | Leu-Leu |
| 11 | 329.1 | iPhe, iTyr | 13 | 29.0 | Tyr-Phe |
| 12 | 297.1 | iMet, iPhe | 14 | 29.3 | Met-Phe |
| 13 | 279.1 | iPhe, iLeu or iIle | 15 | 30.8 | Phe-Leu |
| 14 | 279.1 | iPhe, iLeu or iIle | 16 | 31.5 | Leu-Phe |
| 15 | 313.1 | iPhe | 17 | 33.4 | Phe-Phe |

^{a}EIC peaks are listed by increasing retention times according to FIG. 2.
^{b}Tr is the abbreviation for retention time.
^{c}linear dipeptides were definitely identified by comparing their retention times, their m/z values and their fragmentation patterns with those of reference dipeptides (see Table III).

With reference to FIG. 3 illustrates the MS and MS/MS spectra of the EIC peak 1 detected at 20.6 min during the analysis of the soluble fraction of E. coli cells expressing AlbC. The MS spectrum shows a main m/z peak at 281.0±0.1 (FIG. 3a). This peak was isolated as parent ion and subjected to MS/MS fragmentation giving rise to a daughter ions spectrum (FIG. 3b). Encircled m/z peak at 104.3±0.1 matches to immonium ion of Met, respectively referred to as iMet.

With reference to FIG. 4 illustrates the MS and MS/MS spectra of the EIC peak 2 detected at 22.0 min during the analysis of the soluble fraction of E. coli cells expressing AlbC. The MS spectrum shows a m/z peak at 313.1±0.1 (FIG. 4a). This peak was isolated as parent ion and subjected to MS/MS fragmentation giving rise to a daughter ions spectrum (FIG. 4b). Encircled m/z peak at 136.0±0.1 matches to immonium ion of Tyr, respectively referred to as iTyr and encircled m/z peak at 104.2±0.1 matches to immonium ion of Met referred to as iMet.

With reference to FIG. 5 illustrates the MS and MS/MS spectra of the EIC peak 3 detected at 22.5 min during the analysis of the soluble fraction of E. coli cells expressing AlbC. The MS spectrum shows a m/z peak at 313.1±0.1 (FIG. 5a). This peak was isolated as parent ion and subjected to MS/MS fragmentation giving rise to a daughter ions spectrum (FIG. 5b). Encircled m/z peak at 136.1±0.1 matches to immonium ion of Tyr, respectively referred to as iTyr and encircled m/z peak at 104.3±0.1 matches to immonium ion of Met referred to as iMet.

With reference to FIG. 6 illustrates the MS and MS/MS spectra of the EIC peak 4 detected at 22.9 min during the analysis of the soluble fraction of E. coli cells expressing AlbC. The MS spectrum shows a main m/z peak at 263.0±0.1 (FIG. 6a). This peak was isolated as parent ion and subjected to MS/MS fragmentation giving rise to a daughter ions spectrum (FIG. 6b). Encircled m/z peak at 86.5±0.1 matches to immonium ion of Leu or Ile, respectively referred to as iLeu or iIle and encircled m/z peak at 104.3±0.1 matches to immonium ion of Met referred to as iMet.

With reference to FIG. 7 illustrates the MS and MS/MS spectra of the EIC peak 5 detected at 23.8 min during the analysis of the soluble fraction of E. coli cells expressing AlbC. The MS spectrum shows a minor m/z peak at 295.1±0.1 not detected in the control sample (FIG. 7a). This peak was isolated as parent ion and subjected to MS/MS fragmentation giving rise to a daughter ions spectrum (FIG. 7b). Encircled m/z peak at 136.0±0.1 matches to immonium ion of Tyr referred to as iTyr and encircled m/z peak at 86.6±0.1 matches to immonium ion of Leu or Ile, respectively referred to as iLeu or iIle.

With reference to FIG. 8 illustrates the MS and MS/MS spectra of the EIC peak 6 detected at 25.0 min during the analysis of the soluble fraction of E. coli cells expressing AlbC. The MS spectrum shows a main m/z peak at 263.0±0.1 (FIG. 8a). This peak was isolated as parent ion and subjected to MS/MS fragmentation giving rise to a daughter ions spectrum (FIG. 8b). Encircled m/z peak at 104.2±0.1 matches to immonium ion of Met referred to as iMet and encircled m/z peak at 86.5±0.1 matches to immonium ion of Leu or Ile, respectively referred to as iLeu or iIle.

With reference to FIG. 9 illustrates the MS and MS/MS spectra of the EIC peak 7 detected at 25.9 min during the analysis of the soluble fraction of E. coli cells expressing AlbC. The MS spectrum shows a m/z peak at 295.1±0.1 (FIG. 9a). This peak was isolated as parent ion and subjected to MS/MS fragmentation giving rise to a daughter ions spectrum (FIG. 9b). Encircled m/z peak at 136.1±0.1 matches to immonium ion of Tyr referred to as iTyr and encircled m/z peak at 86.5±0.1 matches to immonium ion of Leu or Ile, respectively referred to as iLeu or iIle.

With reference to FIG. 10 illustrates the MS and MS/MS spectra of the EIC peak 8 detected at 26.6 min during the analysis of the soluble fraction of E. coli cells expressing AlbC. The MS spectrum shows a minor m/z peak at 329.1±0.1 not detected in the control sample (FIG. 10a). This peak was isolated as parent ion and subjected to MS/MS fragmentation giving rise to a daughter ions spectrum (FIG. 10b). Encircled m/z peak at 120.2±0.1 matches to immonium ion of Phe referred to as iPhe and encircled m/z peak at 136.2±0.1 matches to immonium ion of Tyr referred to as iTyr.

With reference to FIG. 11 illustrates the MS and MS/MS spectra of the EIC peak 9 detected at 27.0 min during the analysis of the soluble fraction of E. coli cells expressing AlbC. The MS spectrum shows a m/z peak at 297.1±0.1 (FIG. 11a). This peak was isolated as parent ion and subjected to MS/MS fragmentation giving rise to a daughter ions spectrum (FIG. 11b). Encircled m/z peak at 104.3±0.1 matches to immonium ion of Met referred to as iMet and encircled m/z peak at 120.1±0.1 matches to immonium ion of Phe referred to as iPhe.

With reference to FIG. 12 illustrates the MS and MS/MS spectra of the EIC peak 10 detected at 27.3 min during the analysis of the soluble fraction of E. coli cells expressing AlbC. The MS spectrum shows a main m/z peak at 245.1±0.1 (FIG. 12a). This peak was isolated as parent ion and subjected to MS/MS fragmentation giving rise to a daughter ions spectrum (FIG. 12b). Encircled m/z peak at 86.5±0.1 matches to immonium ion of Leu or Ile, respectively referred to as iLeu or iIle.

With reference to FIG. 13 illustrates the MS and MS/MS spectra of the EIC peak 11 detected at 29.0 min during the analysis of the soluble fraction of E. coli cells expressing AlbC. The MS spectrum shows a m/z peak at 329.1±0.1 not detected in the control sample (FIG. 13a). This peak was isolated as parent ion and subjected to MS/MS fragmentation giving rise to a daughter ions spectrum (FIG. 13b). Encircled m/z peak at 136.1±0.1 matches to immonium ion of Tyr referred to as iTyr and encircled m/z peak at 120.1±0.1 matches to immonium ion of Phe referred to as iPhe.

With reference to FIG. 14 illustrates the MS and MS/MS spectra of the EIC peak 12 detected at 29.3 min during the analysis of the soluble fraction of E. coli cells expressing AlbC. The MS spectrum shows a m/z peak at 297.1±0.1 not detected in the control sample (FIG. 14a). This peak was isolated as parent ion and subjected to MS/MS fragmentation giving rise to a daughter ions spectrum (FIG. 14b). Encircled m/z peak at 120.1±0.1 matches to immonium ion of Phe referred to as iPhe and encircled m/z peak at 104.2±0.1 matches to immonium ion of Met referred to as iMet.

With reference to FIG. 15 illustrates the MS and MS/MS spectra of the EIC peak 13 detected at 30.8 min during the analysis of the soluble fraction of E. coli cells expressing AlbC. The MS spectrum shows a main m/z peak at 279.1±0.1 (FIG. 15a). This peak was isolated as parent ion and subjected to MS/MS fragmentation giving rise to a daughter ions spectrum (FIG. 15b). Encircled m/z peak at 120.1±0.1 matches to immonium ion of Phe referred to as iPhe and encircled m/z peak at 86.5±0.1 matches to immonium ion of Leu or Ile, respectively referred to as iLeu or iIle.

With reference to FIG. 16 illustrates the MS and MS/MS spectra of the EIC peak 14 detected at 31.5 min during the analysis of the soluble fraction of E. coli cells expressing AlbC. The MS spectrum shows a main m/z peak at 279.1±0.1 (FIG. 16a). This peak was isolated as parent ion and subjected to MS/MS fragmentation giving rise to a daughter ions spectrum (FIG. 16b). Encircled m/z peak at 86.5±0.1 matches to immonium ion of Leu or Ile, respectively referred to as iLeu or iIle and encircled m/z peak at 120.2±0.1 matches to immonium ion of Phe referred to as iPhe.

With reference to FIG. 17 illustrates the MS and MS/MS spectra of the EIC peak 15 detected at 33.4 min during the analysis of the soluble fraction of E. coli cells expressing AlbC. The MS spectrum shows a minor m/z peak at 313.1±0.1 not detected in the control sample (FIG. 17a). This peak was isolated as parent ion and subjected to MS/MS fragmentation giving rise to a daughter ions spectrum (FIG. 17b). Encircled m/z peak at 120.2±0.1 matches to immonium ion of Phe referred to as iPhe.

TABLE III
LC-MS/MS analysis reference of chemically-
synthesized dipeptides: summary of data extracted
from figures whose numbers are reported herein.

MS and MS/MS data

| Linear | | Immonium | See FIGS. | LC Data |
|---|---|---|---|---|
| dipeptides^{a} | m/z | ions detected | (n^{o}) | Tr (min)^{b} |
| Met-Met | 281.0 | iMet | 18 | 19.4 |
| Met-Tyr | 313.1 | iMet, iTyr | 19 | 21.6 |
| Ile-Met | 263.0 | iMet, iIle | 20 | 21.8 |
| Tyr-Met | 313.1 | iMet, iTyr | 21 | 22.8 |
| Leu-Met | 263.0 | iLeu, iMet | 22 | 22.9 |
| Ile-Tyr | 295.1 | iIle, iTyr | 23 | 23.3 |
| Tyr-Tyr | 345.1 | iTyr | 24 | 23.5 |
| Leu-Tyr | 295.1 | iLeu, iTyr | 25 | 23.7 |
| Met-Ile | 263.0 | iMet, iIle | 26 | 24.0 |
| Ile-Ile | 245.1 | iIle, iIle | 27 | 24.1 |
| Tyr-Ile | 295.1 | iTyr, iIle | 28 | 24.4 |
| Met-Leu | 263.1 | iMet, iLeu | 29 | 25.3 |
| Leu-Ile | 245.1 | iLeu, iIle | 30 | 25.4 |
| Tyr-Leu | 295.1 | iTyr, iLeu | 31 | 25.8 |
| Ile-Leu | 245.1 | iLeu, iIle | 32 | 26.1 |
| Phe-Tyr | 329.1 | iPhe, iTyr | 33 | 26.7 |
| Phe-Met | 297.1 | iPhe, iMet | 34 | 27.1 |
| Leu-Leu | 245.1 | iLeu | 35 | 27.4 |
| Phe-Ile | 279.1 | iPhe, iIle | 36 | 28.7 |
| Tyr-Phe | 329.1 | iTyr, iPhe | 37 | 29.0 |
| Met-Phe | 297.0 | iMet, iPhe | 38 | 29.5 |
| Ile-Phe | 279.1 | iIle, iPhe | 39 | 30.2 |
| Phe-Leu | 279.1 | iPhe, iLeu | 40 | 30.8 |
| Leu-Phe | 279.1 | iLeu, iPhe | 41 | 31.5 |
| Phe-Phe | 313.1 | iPhe | 42 | 33.4 |

^{a}Linear dipeptides are listed by increasing retention times.
^{b}Tr is the abbreviation for retention time.

With reference to FIG. 18 illustrates the EIC and the MS and MS/MS spectra of the chemically-synthesized Met-Met. An EIC peak is detected at 19.4 minutes (FIG. 18a). The MS spectrum shows a m/z peak at 281.0±0.1 (FIG. 18b). This peak was isolated as parent ion and subjected to MS/MS fragmentation giving rise to a daughter ions spectrum (FIG. 18c). Encircled m/z peak at 104.2±0.1 matches to immonium ion of Met referred to as iMet.

With reference to FIG. 19 illustrates the EIC and the MS and MS/MS spectra of the chemically-synthesized Met-Tyr. An EIC peak is detected at 21.6 minutes (FIG. 19a). The MS spectrum shows a m/z peak at 313.1±0.1 (FIG. 19b). This peak was isolated as parent ion and subjected to MS/MS fragmentation giving rise to a daughter ions spectrum (FIG. 19c). Encircled m/z peak at 136.0±0.1 matches to immonium ion of Tyr referred to as iTyr and encircled m/z peak at 104.2±0.1 matches to immonium ion of Met referred to as iMet.

With reference to FIG. 20 illustrates the EIC and the MS and MS/MS spectra of the chemically-synthesized Ile-Met. An EIC peak is detected at 21.8 minutes (FIG. 20a). The MS spectrum shows a m/z peak at 263.0±0.1 (FIG. 20b). This peak was isolated as parent ion and subjected to MS/MS fragmentation giving rise to a daughter ions spectrum (FIG. 20c). Encircled m/z peak at 86.5±0.1 matches to immonium ion of Ile referred to as iIle and encircled m/z peak at 104.3±0.1 matches to immonium ion of Met referred to as iMet.

With reference to FIG. 21 illustrates the EIC and the MS and MS/MS spectra of the chemically-synthesized Tyr-Met. An EIC peak is detected at 22.8 minutes (FIG. 21a). The MS spectrum shows a m/z peak at 313.1±0.1 (FIG. 21b). This peak was isolated as parent ion and subjected to MS/MS fragmentation giving rise to a daughter ions spectrum (FIG. 21c). Encircled m/z peak at 136.0±0.1 matches to immonium ion of Tyr referred to as iTyr and encircled m/z peak at 104.2±0.1 matches to immonium ion of Met referred to as iMet.

With reference to FIG. 22 illustrates the EIC and the MS and MS/MS spectra of the chemically-synthesized Leu-Met. An EIC peak is detected at 22.9 minutes (FIG. 22a). The MS spectrum shows a m/z peak at 263.0±0.1 (FIG. 22b). This peak was isolated as parent ion and subjected to MS/MS fragmentation giving rise to a daughter ions spectrum (FIG. 22c). Encircled m/z peak at 86.5±0.1 matches to immonium ion of Leu referred to as iLeu and encircled m/z peak at 104.3±0.1 matches to immonium ion of Met referred to as iMet.

With reference to FIG. 23 illustrates the EIC and the MS and MS/MS spectra of the chemically-synthesized Ile-Tyr. An EIC peak is detected at 23.3 minutes (FIG. 23a). The MS spectrum shows a m/z peak at 295.1±0.1 (FIG. 23b). This peak was isolated as parent ion and subjected to MS/MS fragmentation giving rise to a daughter ions spectrum (FIG. 23c). Encircled m/z peak at 86.5±0.1 matches to immonium ion of Ile, referred to as iIle and encircled m/z peak at 136.1±0.1 matches to immonium ion of Tyr referred to as iTyr.

With reference to FIG. 24 illustrates the EIC and the MS and MS/MS spectra of the chemically-synthesized Tyr-Tyr. An EIC peak is detected at 23.5 minutes (FIG. 24a). The MS spectrum shows a m/z peak at 345.1±0.1 (FIG. 24b). This peak was isolated as parent ion and subjected to MS/MS fragmentation giving rise to a daughter ions spectrum (FIG. 24c). Encircled m/z peak at 136.1±0.1 matches to immonium ion of Tyr referred to as iTyr.

With reference to FIG. 25 illustrates the EIC and the MS and MS/MS spectra of the chemically-synthesized Leu-Tyr. An EIC peak is detected at 23.7 minutes (FIG. 25a). The MS spectrum shows a m/z peak at 295.1±0.1 (FIG. 25b). This peak was isolated as parent ion and subjected to MS/MS fragmentation giving rise to a daughter ions spectrum (FIG. 25c). Encircled m/z peak at 86.5±0.1 matches to immonium ion of Leu, referred to as iLeu and encircled m/z peak at 136.1±0.1 matches to immonium ion of Tyr referred to as iTyr.

With reference to FIG. 26 illustrates the EIC and the MS and MS/MS spectra of the chemically-synthesized Met-Ile. An EIC peak is detected at 24.0 minutes (FIG. 26a). The MS spectrum shows a m/z peak at 263.0±0.1 (FIG. 26b). This peak was isolated as parent ion and subjected to MS/MS fragmentation giving rise to a daughter ions spectrum (FIG. 26c). Encircled m/z peak at 104.2±0.1 matches to immonium ion of Met, referred to as iMet and encircled m/z peak at 86.5±0.1 matches to immonium ion of Ile referred to as iIle.

With reference to FIG. 27 illustrates the EIC and the MS and MS/MS spectra of the chemically-synthesized Ile-Ile. An EIC peak is detected at 24.1 minutes (FIG. 27a). The MS spectrum shows a m/z peak at 245.1±0.1 (FIG. 27b). This peak was isolated as parent ion and subjected to MS/MS fragmentation giving rise to a daughter ions spectrum (FIG. 27c). Encircled m/z peak at 86.5±0.1 matches to immonium ion of Ile referred to as iIle.

With reference to FIG. 28 illustrates the EIC and the MS and MS/MS spectra of the chemically-synthesized Tyr-Ile. An EIC peak is detected at 24.4 minutes (FIG. 28a). The MS spectrum shows a m/z peak at 295.1±0.1 (FIG. 28b). This peak was isolated as parent ion and subjected to MS/MS fragmentation giving rise to a daughter ions spectrum (FIG. 28c). Encircled m/z peak at 86.5±0.1 matches to immonium ion of Ile, referred to as iIle and encircled m/z peak at 136.1±0.1 matches to immonium ion of Tyr referred to as iTyr.

With reference to FIG. 29 illustrates the EIC and the MS and MS/MS spectra of the chemically-synthesized Met-Leu. An EIC peak is detected at 25.3 minutes (FIG. 29a). The MS spectrum shows a m/z peak at 263.1±0.1 (FIG. 29b). This peak was isolated as parent ion and subjected to MS/MS fragmentation giving rise to a daughter ions spectrum (FIG. 29c). Encircled m/z peak at 104.2±0.1 matches to immonium ion of Met, referred to as iMet and encircled m/z peak at 86.5±0.1 matches to immonium ion of Leu referred to as iLeu.

With reference to FIG. 30 illustrates the EIC and the MS and MS/MS spectra of the chemically-synthesized Leu-Ile. An EIC peak is detected at 25.4 minutes (FIG. 30a). The MS spectrum shows a m/z peak at 245.1±0.1 (FIG. 30b). This peak was isolated as parent ion and subjected to MS/MS fragmentation giving rise to a daughter ions spectrum (FIG. 30c). Encircled m/z peak at 86.5±0.1 matches to immonium ion of Leu and Ile, respectively referred to as iLeu and iIle.

With reference to FIG. 31 illustrates the EIC and the MS and MS/MS spectra of the chemically-synthesized Tyr-Leu. An EIC peak is detected at 25.8 minutes (FIG. 31a). The MS spectrum shows a m/z peak at 295.1±0.1 (FIG. 31b). This peak was isolated as parent ion and subjected to MS/MS fragmentation giving rise to a daughter ions spectrum (FIG. 31c). Encircled m/z peak at 86.5±0.1 matches to immonium ion of Leu, referred to as iLeu and encircled m/z peak at 136.1±0.1 matches to immonium ion of Tyr referred to as iTyr.

With reference to FIG. 32 illustrates the EIC and the MS and MS/MS spectra of the chemically-synthesized Ile-Leu. An EIC peak is detected at 26.1 minutes (FIG. 32a). The MS spectrum shows a m/z peak at 245.1±0.1 (FIG. 32b). This peak was isolated as parent ion and subjected to MS/MS fragmentation giving rise to a daughter ions spectrum (FIG. 32c). Encircled m/z peak at 86.5±0.1 matches to immonium ions of Ile and Leu, respectively referred to as iIle and iLeu.

With reference to FIG. 33 illustrates the EIC and the MS and MS/MS spectra of the chemically-synthesized Phe-Tyr. An EIC peak is detected at 26.7 minutes (FIG. 33a). The MS spectrum shows a m/z peak at 329.1±0.1 (FIG. 33b). This peak was isolated as parent ion and subjected to MS/MS fragmentation giving rise to a daughter ions spectrum (FIG. 33c). Encircled m/z peak at 120.1±0.1 matches to immonium ion of Phe, referred to as iPhe and encircled m/z peak at 136.1±0.1 matches to immonium ion of Tyr referred to as iTyr.

With reference to FIG. 34 illustrates the EIC and the MS and MS/MS spectra of the chemically-synthesized Phe-Met. An EIC peak is detected at 27.1 minutes (FIG. 34a). The MS spectrum shows a m/z peak at 297.1±0.1 (FIG. 34b). This peak was isolated as parent ion and subjected to MS/MS fragmentation giving rise to a daughter ions spectrum (FIG. 34c). Encircled m/z peak at 120.2±0.1 matches to immonium ion of Phe, referred to as iPhe and encircled m/z peak at 104.3±0.1 matches to immonium ion of Met referred to as iMet.

With reference to FIG. 35 illustrates the EIC and the MS and MS/MS spectra of the chemically-synthesized Leu-Leu. An EIC peak is detected at 27.4 minutes (FIG. 35a). The MS spectrum shows a m/z peak at 245.1±0.1 (FIG. 35b). This peak was isolated as parent ion and subjected to MS/MS fragmentation giving rise to a daughter ions spectrum (FIG. 35c). Encircled m/z peak at 86.5±0.1 matches to immonium ion of Leu referred to as iLeu.

With reference to FIG. 36 illustrates the EIC and the MS and MS/MS spectra of the chemically-synthesized Phe-Ile. An EIC peak is detected at 28.7 minutes (FIG. 36a). The MS spectrum shows a m/z peak at 279.1±0.1 (FIG. 36b). This peak was isolated as parent ion and subjected to MS/MS fragmentation giving rise to a daughter ions spectrum (FIG. 36c). Encircled m/z peak at 120.1±0.1 matches to immonium ion of Phe, referred to as iPhe and encircled m/z peak at 86.5±0.1 matches to immonium ion of Ile referred to as iIle.

With reference to FIG. 37 illustrates the EIC and the MS and MS/MS spectra of the chemically-synthesized Tyr-Phe. An EIC peak is detected at 29.0 minutes (FIG. 37a). The MS spectrum shows a m/z peak at 329.1±0.1 (FIG. 37b). This peak was isolated as parent ion and subjected to MS/MS fragmentation giving rise to a daughter ions spectrum (FIG. 37c). Encircled m/z peak at 120.1±0.1 matches to immonium ion of Phe, referred to as iPhe and encircled m/z peak at 136.1±0.1 matches to immonium ion of Tyr referred to as iTyr.

With reference to FIG. 38 illustrates the EIC and the MS and MS/MS spectra of the chemically-synthesized Met-Phe. An EIC peak is detected at 29.5 minutes (FIG. 38a). The MS spectrum shows a m/z peak at 297.0±0.1 (FIG. 38b). This peak was isolated as parent ion and subjected to MS/MS fragmentation giving rise to a daughter ions spectrum (FIG. 38c). Encircled m/z peak at 120.2±0.1 matches to immonium ion of Phe, referred to as iPhe and encircled m/z peak at 104.3±0.1 matches to immonium ion of Met referred to as iMet.

With reference to FIG. 39 illustrates the EIC and the MS and MS/MS spectra of the chemically-synthesized Ile-Phe. An EIC peak is detected at 30.2 minutes (FIG. 39a). The MS spectrum shows a m/z peak at 279.1±0.1 (FIG. 39b). This peak was isolated as parent ion and subjected to MS/MS fragmentation giving rise to a daughter ions spectrum (FIG. 39c). Encircled m/z peak at 86.5±0.1 matches to immonium ion of Ile, referred to as iIle and encircled m/z peak at 120.2±0.1 matches to immonium ion of Phe referred to as iPhe.

With reference to FIG. 40 illustrates the EIC and the MS and MS/MS spectra of the chemically-synthesized Phe-Leu. An EIC peak is detected at 30.8 minutes (FIG. 40a). The MS spectrum shows a m/z peak at 279.1±0.1 (FIG. 40b). This peak was isolated as parent ion and subjected to MS/MS fragmentation giving rise to a daughter ions spectrum (FIG. 40c). Encircled m/z peak at 86.5±0.1 matches to immonium ion of Leu, referred to as iLeu and encircled m/z peak at 120.1±0.1 matches to immonium ion of Phe referred to as iPhe.

With reference to FIG. 41 illustrates the EIC and the MS and MS/MS spectra of the chemically-synthesized Leu-Phe. An EIC peak is detected at 31.5 minutes (FIG. 41a). The MS spectrum shows a m/z peak at 279.1±0.1 (FIG. 41b). This peak was isolated as parent ion and subjected to MS/MS fragmentation giving rise to a daughter ions spectrum (FIG. 41c). Encircled m/z peak at 86.5±0.1 matches to immonium ion of Leu, referred to as iLeu and encircled m/z peak at 120.2±0.1 matches to immonium ion of Phe referred to as iPhe.

With reference to FIG. 42 illustrates the EIC and the MS and MS/MS spectra of the chemically-synthesized Phe-Phe. An EIC peak is detected at 33.4 minutes (FIG. 42a). The MS spectrum shows a m/z peak at 313.1±0.1 (FIG. 42b). This peak was isolated as parent ion and subjected to MS/MS fragmentation giving rise to a daughter ions spectrum (FIG. 42c). Encircled m/z peak at 120.1±0.1 matches to immonium ion of Phe referred to as iPhe.

2) Linear Dipeptides Produced in the Presence of Rv2275.

The soluble fraction of E. coli cells expressing Rv2275-his (SEQ ID NO:36) was analyzed by LC-MS as previously described. This analysis which leads to one set of EICs was compared to that of the control experiment using cells transformed with a vector not coding for a CDS. This comparison showed one significant EIC peak matching with a linear dipeptide and being specific to Rv2275 activity (FIG. 43 and FIG. 44 specified in Table IV).

TABLE IV
LC-MS/MS analysis of the soluble fraction of E. coli cells
expressing Rv2275: summary of data extracted from figure
whose number is reported herein and identification of
linear dipeptide.

| | MS and MS/MS data | | | | |
|---|---|---|---|---|---|
| EIC | | immonium | See Figure | LC Data | Identified |
| Peak^{a} | m/z | ion detected | (n^{o}) | Tr (min)^{b} | dipeptides^{c} |
| 1 | 345.1 | iTyr | 44 | 23.3 | Tyr-Tyr |

^{a}EIC peak listed named according to FIG. 43.
^{b}Tr is the abbreviation for retention time.
^{c}linear dipeptide was definitely identified by comparing its retention time, its m/z value and its fragmentation pattern with those of reference dipeptides (see Table III).

With reference to FIG. 43 illustrates EICs of dipeptides m/z values specific to Rv2275 and detected from a LCMS analysis of the soluble fraction of E. coli cells expressing Rv2275 (upper black traces) compared to the same set of EICs from a LCMS analysis of the control sample (lower grey traces). The only significant specific EIC peak was labeled as specified in Table IV for identification by MS and MS/MS illustrated in the FIG. 44.

With reference to FIG. 44 illustrates the MS and MS/MS spectra of the EIC peak 1 detected at 23.3 min during the analysis of the soluble fraction of E. coli cells expressing Rv2275. The MS spectrum shows a m/z peak at 345.1±0.1 not detected in the control sample (FIG. 44a). This peak was isolated as parent ion and subjected to MS/MS fragmentation giving rise to a daughter ions spectrum (FIG. 44b). Encircled m/z peak at 136.1±0.1 matches to immonium ion of Tyr referred to as iTyr.

This EIC peak was further characterized by MS/MS fragmentation and the analysis of the daughter ions spectrum, this enabled the identification of one potential matching linear dipeptide, namely Tyr-Tyr (Table IV). The comparison of its retention time and its fragmentation pattern with those of reference chemically-synthesized Tyr-Tyr (see Table III and FIG. 24) allowed the Inventors to conclude that the expression of Rv2275 in E. coli cells is responsible for the in vivo formation of Tyr-Tyr (see Table IV).

3) Linear Dipeptides Produced in the Presence of YvmC-Bsub.

The soluble fraction of E. coli cells expressing YvmC-Bsub-his (SEQ ID NO:37) was analyzed by LC-MS as previously described. The analysis which leads to one set of EICs is compared to that of a control experiment using cells transformed with a vector not expressing CDS. This comparison enabled the Inventors to detect 12 EIC peaks matching with linear dipeptides and being specific to the YvmC-Bsub activity (FIG. 45 and Figures specified in Table V).

TABLE V
LC-MS/MS analysis of the soluble fraction of E. coli cells
expressing YvmC-Bsub: summary of data extracted from
figures whose numbers are reported herein and identification
of linear dipeptides.

| | MS and MS/MS data | | | | |
|---|---|---|---|---|---|
| | | | See | | |
| EIC | | immonium | Figures | LC Data | Identified |
| Peaks^{a} | M/z | ions detected | (n^{o}) | Tr (min)^{b} | dipeptides^{c} |
| 1 | 281.0 | iMet | 46 | 20.6 | Met-Met |
| 2 | 263.1 | iMet, iLeu or iIle | 47 | 21.8 | Ile-Met |
| 3 | 263.0 | iMet, iLeu or iIle | 48 | 22.8 | Leu-Met |
| 4 | 263.0 | iMet, iLeu or iIle | 49 | 24.9 | Met-Leu |
| 5 | 245.1 | iLeu or iIle | 50 | 25.4 | Leu-Ile |
| 6 | 245.1 | iLeu or iIle | 51 | 25.9 | Ile-Leu |
| 7 | 297.0 | iMet, iPhe | 52 | 26.8 | Phe-Met |
| 8 | 245.1 | iLeu or iIle | 53 | 27.3 | Leu-Leu |
| 9 | 297.0 | iMet, iPhe | 54 | 29.2 | Met-Phe |
| 10 | 279.1 | iPhe, iLeu ou iIle | 55 | 30.8 | Phe-Leu |
| 11 | 279.1 | iPhe, iLeu ou iIle | 56 | 31.4 | Leu-Phe |
| 12 | 313.1 | iPhe | 57 | 33.3 | Phe-Phe |

^{a}EIC peaks are listed by increasing retention times according to FIG. 45.
^{b}Tr is the abbreviation for retention time.
^{c}linear dipeptides were definitely identified by comparing their retention times, their m/z values and their fragmentation patterns with those of reference dipeptides (see Table III).

With reference to FIG. 45 illustrates EICs of dipeptides m/z values specific to YvmC and detected from a LCMS analysis of the soluble fraction of E. coli cells expressing YvmC (upper black traces) compared to the same set of EICs from a LCMS analysis of the control sample (lower grey traces). A close-up view is made to distinguish the minor products detected in the sample. The specific EIC peaks were labeled as specified in Table V for identification by MS and MS/MS illustrated in the FIGS. 46 to 57.

With reference to FIG. 46 illustrates the MS and MS/MS spectra of the EIC peak 1 detected at 20.6 min during the analysis of the soluble fraction of E. coli cells expressing YvmC. The MS spectrum shows a main m/z peak at 281.0±0.1 not detected in the control sample (FIG. 46a). This peak was isolated as parent ion and subjected to MS/MS fragmentation giving rise to a daughter ions spectrum (FIG. 46b). Encircled m/z peak at 104.3±0.1 matches to immonium ion of Met, respectively referred to as iMet.

With reference to FIG. 47 illustrates the MS and MS/MS spectra of the EIC peak 2 detected at 21.8 min during the analysis of the soluble fraction of E. coli cells expressing YvmC. The MS spectrum shows a m/z peak at 263.1±0.1 not detected in the control sample (FIG. 47a). This peak was isolated as parent ion and subjected to MS/MS fragmentation giving rise to a daughter ions spectrum (FIG. 47b). Encircled m/z peak at 86.5±0.1 matches to immonium ion of Leu or Ile, respectively referred to as iLeu or iIle and encircled m/z peak at 104.3±0.1 matches to immonium ion of Met referred to as iMet.

With reference to FIG. 48 illustrates the MS and MS/MS spectra of the EIC peak 3 detected at 22.8 min during the analysis of the soluble fraction of E. coli cells expressing YvmC. The MS spectrum shows a main m/z peak at 263.0±0.1 (FIG. 48a). This peak was isolated as parent ion and subjected to MS/MS fragmentation giving rise to a daughter ions spectrum (FIG. 48b). Encircled m/z peak at 86.5±0.1 matches to immonium ion of Leu or Ile, respectively referred to as iLeu or iIle and encircled m/z peak at 104.2±0.1 matches to immonium ion of Met referred to as iMet.

With reference to FIG. 49 illustrates the MS and MS/MS spectra of the EIC peak 4 detected at 24.9 min during the analysis of the soluble fraction of E. coli cells expressing YvmC. The MS spectrum shows a main m/z peak at 263.0±0.1 (FIG. 49a). This peak was isolated as parent ion and subjected to MS/MS fragmentation giving rise to a daughter ions spectrum (FIG. 49b). Encircled m/z peak at 104.2±0.1 matches to immonium ion of Met referred to as iMet and encircled m/z peak at 86.5±0.1 matches to immonium ion of Leu or Ile, respectively referred to as iLeu or iIle.

With reference to FIG. 50 illustrates the MS and MS/MS spectra of the EIC peak 5 detected at 25.4 min during the analysis of the soluble fraction of E. coli cells expressing YvmC. The MS spectrum shows a m/z peak at 245.1±0.1 not detected in the control sample (FIG. 50a). This peak was isolated as parent ion and subjected to MS/MS fragmentation giving rise to a daughter ions spectrum (FIG. 50b). Encircled m/z peak at 86.5±0.1 matches to immonium ion of Leu or Ile, respectively referred to as iLeu or iIle.

With reference to FIG. 51 illustrates the MS and MS/MS spectra of the EIC peak 6 detected at 25.9 min during the analysis of the soluble fraction of E. coli cells expressing YvmC. The MS spectrum shows a main m/z peak at 245.1±0.1 (FIG. 51a). This peak was isolated as parent ion and subjected to MS/MS fragmentation giving rise to a daughter ions spectrum (FIG. 51b). Encircled m/z peak at 86.5±0.1 matches to immonium ion of Leu or Ile, respectively referred to as iLeu or iIle.

With reference to FIG. 52 illustrates the MS and MS/MS spectra of the EIC peak 7 detected at 26.8 min during the analysis of the soluble fraction of E. coli cells expressing YvmC. The MS spectrum shows a main m/z peak at 297.0±0.1 (FIG. 52a). This peak was isolated as parent ion and subjected to MS/MS fragmentation giving rise to a daughter ions spectrum (FIG. 52b). Encircled m/z peak at 120.2±0.1 matches to immonium ion of Phe referred to as iPhe and encircled m/z peak at 104.3±0.1 matches to immonium ion of Met, respectively referred to as iMet.

With reference to FIG. 53 illustrates the MS and MS/MS spectra of the EIC peak 8 detected at 27.3 min during the analysis of the soluble fraction of E. coli cells expressing YvmC. The MS spectrum shows a main m/z peak at 245.1±0.1 (FIG. 53a). This peak was isolated as parent ion and subjected to MS/MS fragmentation giving rise to a daughter ions spectrum (FIG. 53b). Encircled m/z peak at 86.5±0.1 matches to immonium ion of Leu or Ile, respectively referred to as iLeu or iIle.

With reference to FIG. 54 illustrates the MS and MS/MS spectra of the EIC peak 9 detected at 29.2 min during the analysis of the soluble fraction of E. coli cells expressing YvmC. The MS spectrum shows a m/z peak at 297.0±0.1 (FIG. 54a). This peak was isolated as parent ion and subjected to MS/MS fragmentation giving rise to a daughter ions spectrum (FIG. 54b). Encircled m/z peak at 120.1±0.1 matches to immonium ion of Phe referred to as iPhe and encircled m/z peak at 104.2±0.1 matches to immonium ion of Met, respectively referred to as iMet.

With reference to FIG. 55 illustrates the MS and MS/MS spectra of the EIC peak 10 detected at 30.8 min during the analysis of the soluble fraction of E. coli cells expressing YvmC. The MS spectrum shows a m/z peak at 279.1±0.1 (FIG. 55a). This peak was isolated as parent ion and subjected to MS/MS fragmentation giving rise to a daughter ions spectrum (FIG. 55b). Encircled m/z peak at 120.1±0.1 matches to immonium ion of Phe referred to as iPhe and encircled m/z peak at 86.5±0.1 matches to immonium ion of Leu or Ile, respectively referred to as iLeu or iIle.

With reference to FIG. 56 illustrates the MS and MS/MS spectra of the EIC peak 11 detected at 31.4 min during the analysis of the soluble fraction of E. coli cells expressing YvmC. The MS spectrum shows a m/z peak at 279.1±0.1 (FIG. 56a). This peak was isolated as parent ion and subjected to MS/MS fragmentation giving rise to a daughter ions spectrum (FIG. 56b). Encircled m/z peak at 86.5±0.1 matches to immonium ion of Leu or Ile, respectively referred to as iLeu or iIle and encircled m/z peak at 120.1±0.1 matches to immonium ion of Phe referred to as iPhe.

With reference to FIG. 57 illustrates the MS and MS/MS spectra of the EIC peak 12 detected at 33.3 min during the analysis of the soluble fraction of E. coli cells expressing YvmC. The MS spectrum shows a minor m/z peak at 313.1±0.1 not detected in the control sample (FIG. 57a). This peak was isolated as parent ion and subjected to MS/MS fragmentation giving rise to a daughter ions spectrum (FIG. 57b). Encircled m/z peak at 120.1±0.1 matches to immonium ion of Phe referred to as iPhe.

All these EIC peaks, except peak 1, peak 7, peak 9 and peak 12, correspond to linear dipeptides containing the isomass leucyl or isoleucyl residues (Table V and figures numbered herein).

Finally, the comparison of the retention times and fragmentation patterns of the 12 linear dipeptides with those of reference chemically-synthesized dipeptides (see Table III and figures numbered herein) allowed the Inventors to conclude that the expression of YvmC-Bsub in E. coli cells is responsible for the in vivo formation of the following dipeptides: Ile-Met, Leu-Met, Met-Leu, Leu-Ile, Ile-Leu, Leu-Leu, Phe-Leu, Leu-Phe, Phe-Phe, Met-Met, Phe-Met and Met-Phe (see Table V). The two possible sequences of each detected linear dipeptides were always observed except for Ile-Met as its counterpart Met-Ile was not identified. It is reasonably supposed that Met-Ile was also produced by YvmC-Bsub but its quantity was too small to be detected.

In conclusion, the three tested CDSs (namely AlbC, Rv2275 and YvmC-Bsub) can be used to produce linear dipeptides when introduced in bacterial cells such as E. coli cells. However all CDSs which meet the criteria specified above are able to direct the in vivo synthesis of linear dipeptides.

### EXAMPLE 3

### Isolation of a New CDS Coding Sequence by a PCR-Based Approach

As indicated previously Streptomyces noursei and Streptomyces albulus synthesize albonoursin. Streptomyces sp IMI 351155 has been reported to synthesize 1-N-methylalbonoursin (Biosynthesis of 1-N-methylalbonoursin by an endophytic Streptomyces sp. Isolated from perennial ryegrass, Gurney and Mantle, J. Nat. Prod. 1993, 56:1194-1198). The Inventors have also found that this strain produces albonoursin, in addition to 1-N-methylalbonoursin.

The Inventors sought to identify the existence of one or more CDS homologous genes in this strain.

The Inventors first performed hybridization experiments under stringent or non stringent conditions, but these did not allow them to detect any fragment in the genomic DNA of Streptomyces sp IMI 351155 hybridizing with a probe corresponding to the gene albC, or with probes corresponding to other alb genes (e.g. albA and albB,) from Streptomyces noursei.

It should be noted that the same type of hybridization experiments performed with total genomic DNA of Streptomyces albulus revealed DNA fragments hybridizing under stringent conditions. Further isolation and characterization of these fragments from Streptomyces albulus genomic DNA confirmed that they contained the genes directing albonoursin and linear dipeptide biosynthesis.

A Polymerase Chain Reaction (PCR) based approach was therefore developed to find and isolate the albC homologue from Streptomyces sp IMI 351155, i.e. the gene responsible for linear dipeptide biosynthesis.

To design the primers for this PCR-based reaction, the Inventors used the two regions containing the conserved amino acid motifs in all the know CDSs, corresponding to SEQ ID NO:9 and SEQ ID NO:10. However to limit the degeneracy of the primers, the Inventors took into account the partial conservation at some positions, even if this was not taken in account in the definition of the signature H-X-[LVI]-[LVI]-G-[LVI]-S (SEQ ID NO:9) and Y-[LVI]-X-X-E-X-P (SEQ ID NO:10).

The primers were designed from the sequences H-[LVA]-[LVI]-[LVI]-G-[VI]-S (SEQ ID NO:24) and Y-[VI]-[LICF]-[AD]-E-[ALI]-P-[LFA]-[FY] (SEQ ID NO:25, see FIGS. 59 and 60).

A part of the alignment of all CDSs sequences in the first motif are shown in FIG. 59 and the region used for primer design is indicated by a line under the alignment. The numbering is that of AlbC from S. noursei. The degenerated amino acid sequence is shown with the corresponding nucleotide sequence. The first primer was finalised as:

| | |
|---|---|
| 5′ CAC BYS NTS NTS GGS RTS WSS SC | (SEQ ID NO: 22) |

In which for nucleotide: B=C or G or T, N=A or C or G or T, R=A or G, S=C or G, W=A or T, Y=C or T.

A part of the alignment of all CDSs sequences in the second motif are shown in FIG. 60 and the region used for primer design is indicated by a line under the alignment. The numbering is that of AlbC from S. noursei. The degenerated amino acid sequence is shown with the corresponding nucleotide sequence, and the complementary strand (at the bottom) used as primer. The second primer was finalized as:

(SEQ ID NO: 23)
5′ ATG YAS DMS CKS CTC NRS GGS MRS AWG

In which for nucleotide: D=A or G or T, K=G or T, M=A or C, N=A or C or G or T, R=A or G, S=C or G, W=A or T, Y=C or T.

To reduce the degeneracy of the primers, the codon usage of Streptomyces was taken into account. As the genomic DNA of Streptomyces is GC rich, the third position in all codons is preferentially a C or G. Therefore, in the primers, all nucleotides corresponding to the third position in a codon were modified to either C or G, for example residues in the primer Y became C, and residues N became S). The two degenerated primers used were Primer 1 5′-CACBYSNTSNTSGGSRTSWSSSC-3′ (SEQ ID NO:26) and Primer 2 5′-GWASRMSGGSRNCTCSKCSMDSAYGTA-3′ (SEQ ID NO:27).

PCR using these primers was performed on cDNA obtained by reverse transcription of the total RNA extracted from Streptomyces sp. IMI 351155 after 3 days of cultivation in HT medium. This time of cultivation correspond to the onset of dipeptide biosynthesis, a time where the dipeptide biosynthetic genes should be transcribed. Total RNA was extracted using well established protocols and cDNAs were obtained using the kit SuperScript® First-Strand Synthesis System for RT-PCR from Invitrogen.

To enhance the specificity of the PCR reaction, ramping PCR conditions were used as follows: after an initial denaturation step at 95° C. for 2 min, the annealing temperature was initially 37° C., and it was increased to 72° C. in steps of 1° C. every 15 s. This was followed by denaturation at 95° C. for 30 s. Two such cycles were performed. Then the PCR program consisted of 35 cycles of 95° C. for 30 s, 55° C. for 1 min 30 s and 72° C. for 1 min. Taq polymerase was used.

The PCR products obtained were separated by agarose gel electrophoresis. A faint band of about 470 by was visible. DNA in the range 450-500 by was extracted from the gel and a fraction was used as template for PCR amplification with primer 1 and 2. The PCR program consisted of an initial denaturation step at 95° C. for 2 min, followed by 35 cycles of 95° C. for 30 s, 55° C. for 1 min 30 s and 72° C. for 1 min. Taq polymerase was used. The PCR products were separated by agarose gel electrophoresis. A band of about 470 by was clearly visible. This band was extracted from the gel and ligated to the vector pGEMT-Easy (Promega). The ligation mix was used to transform competent E. coli cells. Plasmids were extracted from nine clones and the nucleotide sequence of their inserts was determined. All the inserts were very similar, the differences between them being in the region corresponding to the two degenerated primers. The deduced products were similar to AlbC from Streptomyces noursei (42% identity in amino acids).

To obtain the complete albC homolgue from Streptomyces sp. IMI351155 (called thereafter albC-IMI), a gene library of the genomic DNA from Streptomyces sp. IMI351155 was constructed in the cosmid pWED2 (Karray et al. 2007, Organization of the biosynthetic gene cluster for the macrolide antibiotic spiramycin in Streptomyces ambofaciens, Microbiology, in press). The cloned PCR fragment, corresponding to part of the albC-IMI gene, was used as a probe in a colony hybridization experiment. This led to the isolation of 4 clones which hybridized strongly with the probe. The cosmids that they contained were extracted and shown to have fragments in their inserts which hybridized with the albC-IMI probe.

These fragments were subcloned and their nucleotide sequences were determined. This led to the characterization of three genes albA-IMI, albB-IMI and albC-IMI encoding proteins which present respectively 51%; 50% and 40% amino acid identity with AlbA, AlbB and AlbC from Streptomyces noursei.

1-34. (canceled)

35. A method for the production of a linear dipeptide, characterized in that comprising the steps:
a) culturing upon a medium a host cell which has the ability to produce a protein or an active fragment thereof having the activity to form a linear dipeptide from one or more kinds of amino acids;
b) allowing said linear dipeptide to form and accumulate in said host cell and optionally in said medium;
c) recovering said linear dipeptide from an extract of said host cell and optionally said medium;
wherein said protein or an active fragment thereof is selected in the group consisting of proteins and fragments thereof, having at least 20% identity and no more than 90% identity with SEQ ID NO:1.

36. A method for the production of linear dipeptide, according to claim 35, wherein said protein or an active fragment thereof is encoded by an endogenous gene of said host cell.

37. A method for the production of linear dipeptide, according to claim 35, wherein said protein or an active fragment thereof is not encoded by an endogenous gene of said host cell.

38. A method for the production of linear dipeptide, according to claim 35, wherein said host cell comprises coding sequences for at least two proteins or active fragments thereof.

39. A method for the production of linear dipeptide, according to claim 35, wherein said at least two coding sequences come from different genes.

40. A method for the production of linear dipeptide, according to claim 35, wherein said at least two coding sequences come from a single gene.

41. A method for the production of linear dipeptide according to claim 35, wherein said protein or an active fragment thereof has at least 20% and no more than 35% identity with SEQ ID NO:1.

42. A method for the production of linear dipeptide, according to claim 35, wherein said protein or an active fragment thereof comprises a first conserved amino acid sequence of the general sequence SEQ ID NO:9:
H-X-[LVI]-[LVI]-G-[LVI]-S (SEQ ID NO:9)
wherein H=histidine, X=any amino acid, [LVI]=any one of leucine, valine or isoleucine, G=glycine and S=serine.

43. A method for the production of linear dipeptide, according to claim 35, wherein said protein or an active fragment thereof comprises a second conserved amino acid sequence of the general sequence SEQ ID NO:10:

| | |
|---|---|
| Y-[LVI]-X-X-E-X-P | (SEQ ID NO: 10) |

wherein Y=tyrosine, [LVI]=any one of leucine, valine or isoleucine, X=any amino acid, E=glutamic acid and P=proline.

44. A method for the production of linear dipeptide, according to claim 42, wherein said first conserved amino acid sequence and said second amino acid sequence are separated by at least 120 amino acid residues and no more than 160 amino acid residues.

45. A method for the production of linear dipeptide, according to claim 43, wherein said first conserved amino acid sequence and said second amino acid sequence are separated by at least 140 amino acid residues and no more than 150 amino acid residues.

46. A method for the production of linear dipeptide, according to claim 42, wherein said first conserved amino acid sequence corresponds to residues 31 to 37 of SEQ ID NO:1.

47. A method for the production of linear dipeptide, according to claim 43, wherein said second conserved amino acid sequence corresponds to residues 178 to 184 of SEQ ID NO:1.

48. A method for the production of linear dipeptide, according to claim 35, wherein said protein or an active fragment thereof was isolated from a microorganism belonging to the genus Bacillus, Corynebacterium, Mycobacterium, Streptomyces, Photorhabdus or Staphylococcus.

49. A method for the production of linear dipeptide, according to claim 35, wherein said protein or an active fragment thereof was isolated from a microorganism selected from the list Bacillus licheniformis, Bacillus subtilis subsp. subtilis, Bacillus thuringiensis serovar israelensis, Photorhabdus luminescens subsp. laumondii, Staphylococcus haemolyticus, Corynebacterium jeikeium, Mycobacterium tuberculosis, Mycobacterium bovis or Mycobacterium bovis BCG.

50. A method for the production of linear dipeptide, according to claim 35, wherein said protein or an active fragment thereof is selected from the group consisting of AlbC (SEQ ID NO:1), Rv2275 (SEQ ID NO:2), MT2335 (SEQ ID NO:2), MRA2294 (SEQ ID NO:2), TBFG12300 (SEQ ID NO:2), Mb2298 (SEQ ID NO:2), BCG2292 (SEQ ID NO:34), YvmC-Bsub (SEQ ID NO:3), YvmClic (SEQ ID NO:4), YvmC-Bthu (SEQ ID NO:5), pSHaeCO06 (SEQ ID NO:6), Plu0297 (SEQ ID NO:7), JK0923 (SEQ ID NO:8), AlbC-his (SEQ ID NO:35), Rv2275-his (SEQ ID NO:36), YvmC-Bsub-his (SEQ ID NO:37).

51. A method for the production of linear dipeptide, according to claim 35, wherein said linear dipeptide is selected from the group: Phe-Leu, Leu-Phe, Phe-Phe, Phe-Tyr, Tyr-Phe, Leu-Leu, Leu-Tyr, Tyr-Leu, Phe-Met, Met-Phe, Leu-Met, Met-Leu, Tyr-Met, Met-Tyr, Met-Met, Tyr-Tyr, Ile-Met, Met-Ile, Leu-Ile, Ile-Leu.

52. A method for the production of linear dipeptide, wherein said protein or an active fragment thereof is encoded by an isolated, natural or synthetic nucleic acid sequence coding selected from the group consisting of SEQ ID NO:11, SEQ ID NO:12, SEQ ID NO:13, SEQ ID NO:14, SEQ ID NO:15, SEQ ID NO:16, SEQ ID NO:20, SEQ ID NO:21, positions 114-861 of SEQ ID NO:17, positions 114-1008 of SEQ ID NO:18 and positions 114-885 of SEQ ID NO:19.

53. A recombinant vector comprising a nucleic acid coding sequence as claimed in claim 52, wherein said vector is configured to introduce said nucleic acid coding sequence into at least one host cell and said coding sequence is thereby expressed by the endogenous expression mechanisms of said host cell.

54. A recombinant vector comprising a nucleic acid coding sequence as claimed in claim 53, wherein said recombinant vector is selected from the group comprising SEQ ID NO:17, SEQ ID NO:18 and SEQ ID NO:19.

55. A recombinant vector, as claimed in claim 53, wherein said recombinant vector comprises coding sequences for at least two proteins or active fragments thereof.

56. A recombinant vector, as claimed in claim 53, wherein said at least two coding sequences come from different genes.

57. A recombinant vector, as claimed in claim 53, wherein said at least two coding sequences come from a single gene.

58. A recombinant vector, as claimed in claim 53, wherein said host cell is a prokaryote.

59. A recombinant vector, as claimed in claim 53, wherein said host cell is Escherichia coli.

60. A recombinant vector comprising said nucleic acid coding sequence as claimed in claim 52, wherein said vector is configured to express said nucleic acid coding sequence in a cell free expression system by the endogenous transcription mechanisms of said cell free expression system.

61. A method for the production of a linear dipeptide, characterized in that it comprises the steps:
a) inducing a cell free expression system to produce a protein or an active fragment thereof, having the activity to form a dipeptide from one or more kinds of amino acids;
b) introducing at least one amino acid substrate to said protein or an active fragment thereof;
c) allowing said dipeptide to form and accumulate;
d) recovering said dipeptide;
wherein said protein or an active fragment thereof is selected in the group consisting proteins and fragments thereof, having at least 20% identity and no more than 90% identity with SEQ ID NO:1.

62. A method of identifying polypeptides that catalyse the formation of a linear dipeptide of the general formula (i):
R¹-R² (i)
(wherein R¹ and R², which may be the same or different and each may represent any amino acid);
characterized in that it comprises the steps:
a) identifying a candidate polypeptide sequence as having at least one of the following motifs:

| | |
|---|---|
| H-X-[LVI]-[LVI]-G-[LVI]-S | (SEQ ID NO: 9) |

wherein H=histidine, X=any amino acid, [LVI]=any one of leucine, valine or isoleucine, G=glycine and S=serine; and
wherein at least one of said H, LVI, G or S can be another amino acid namely H can be replaced by any one of Lysine or Arginine; LVI can be replaced by any one of Glycine, Alanine, Leucine, Valine or Isoleucine; G can be replaced by any one of Glycine, Alanine, Leucine, Valine or Isoleucine; S can be replaced by Cysteine, Threonine or Methionine.

| | |
|---|---|
| Y-[LVI]-X-X-E-X-P | (SEQ ID NO: 10) |

wherein Y=tyrosine, [LVI]=any one of leucine, valine or isoleucine, X=any amino acid, E=glutamic acid and P=proline; and
wherein at least one of said Y, LVI, E, X or P can be another amino acid namely Y can be replaced by any one of Phenylalanine or Trytophan; LVI can be replaced by any one of Glycine, Alanine, Leucine, Valine or Isoleucine; E can be replaced by any one of Aspartic Acid, Asparagine, Glutamine; P can be replaced by any one of Glycine, Alanine, Leucine, Valine or Isoleucine;
b) creating a polypeptide expression construct by linking said candidate polypeptide coding sequence to promoter sequences configured to express said candidate peptide at an appreciable level;
c) introducing said polypeptide expression construct into at least one cell and inducing the take up of said polypeptide expression construct by said at least one cell or a cell free expression system;
d) monitoring the levels and types of linear dipeptides in the growth medium of said at least one cell or said cell free expression system;
e) comparing the levels of linear dipeptides in the presence of said polypeptide expression construct to the levels of linear dipeptides in the absence of said polypeptide expression construct to determine the relative level of production of linear dipeptides by said polypeptide expression construct; and
f) correlating the relative production of linear dipeptides to expression of said candidate polypeptide in said at least one cell or said cell free expression system.

63. A method of identifying polypeptides that catalyse the formation of a linear dipeptide of the general formula (i):
R¹-R² (i)
(wherein R¹ and R², which may be the same or different and each may represent any amino acid);
characterized in that it comprises the steps:
a) identifying a candidate polypeptide sequence as having both of the following motifs:

| | |
|---|---|
| H-X-[LVI]-[LVI]-G-[LVI]-S | (SEQ ID NO: 9) |

wherein H=histidine, X=any amino acid, [LVI]=any one of leucine, valine or isoleucine, G=glycine and S=serine; and
wherein at least one of said H, LVI, G or S can be another amino acid namely H can be replaced by any one of Lysine or Arginine; LVI can be replaced by any one of Glycine, Alanine, Leucine, Valine or Isoleucine; G can be replaced by any one of Glycine, Alanine, Leucine, Valine or Isoleucine; S can be replaced by Cysteine, Threonine or Methionine.

| | |
|---|---|
| Y-[LVI]-X-X-E-X-P | (SEQ ID NO: 10) |

wherein Y=tyrosine, [LVI]=any one of leucine, valine or isoleucine, X=any amino acid, E=glutamic acid and P=proline; and
wherein at least one of said Y, LVI, E, X or P can be another amino acid namely Y can be replaced by any one of Phenylalanine or Trytophan; LVI can be replaced by any one of Glycine, Alanine, Leucine, Valine or Isoleucine; E can be replaced by any one of Aspartic Acid, Asparagine, Glutamine; P can be replaced by any one of Glycine, Alanine, Leucine, Valine or Isoleucine;
b) creating a polypeptide expression construct by linking said candidate polypeptide coding sequence to promoter sequences configured to express said candidate peptide at an appreciable level;
c) introducing said polypeptide expression construct into at least one cell and inducing the take up of said polypeptide expression construct by said at least one cell or a cell free expression system;
d) monitoring the levels and types of linear dipeptides in the growth medium of said at least one cell or said cell free expression system;
e) comparing the levels of linear dipeptides in the presence of said polypeptide expression construct to the levels of linear dipeptides in the absence of said polypeptide expression construct to determine the relative level of production of linear dipeptides by said polypeptide expression construct; and
f) correlating the relative production of linear dipeptides to expression of said candidate polypeptide in said at least one cell or said cell free expression system.

64. A method for identifying polypeptides according to claim 63, wherein said first conserved motif (SEQ ID NO:9) and said second conserved motif (SEQ ID NO:10) are separated by at least 75 and no more than 250 amino acids.

65. A method for identifying polypeptides according to claim 63, wherein said first conserved motif (SEQ ID NO:9) and/or said second conserved motif (SEQ ID NO:10) comprise more than one residue change.

66. A method for identifying polypeptides according to claim 63, wherein step
a) of said method comprises the amplification of candidate peptide coding nucleic acid sequences using degenerated primers of SEQ ID NO:22 and SEQ ID NO:23 in a Polymerase Chain Reaction.

67. A method of identifying polypeptides that catalyse the formation of a linear dipeptide of the general formula (i):
R¹-R² (i)
wherein R¹ and R², which may be the same or different and each may represent any amino acid;
characterized in that it comprises the steps:
a) identifying a candidate polypeptide sequence as having at least 20% identity and no more than 90% identity with SEQ ID NO:1; or having at least 20% identity with any one of SEQ ID NO:2, SEQ ID NO:3, SEQ ID NO:4, SEQ ID NO:5, SEQ ID NO:6, SEQ ID NO:7, SEQ ID NO:8, SEQ ID NO:35, SEQ ID NO:36, SEQ ID NO:37;
b) creating a polypeptide expression construct by linking said candidate polypeptide sequence to promoter sequences configured to express said candidate peptide at an appreciable level;
c) introducing said polypeptide expression construct into at least one cell and inducing the take up of said polypeptide expression construct by said at least one cell or a cell free expression system;
d) monitoring the levels and types of linear dipeptides in the growth medium of said at least one cell or said cell free expression system;
e) comparing the levels of linear dipeptides in the presence of said polypeptide expression construct to the levels of linear dipeptides in the absence of said polypeptide expression construct to determine the relative level of production of linear dipeptides by said polypeptide expression construct; and
f) correlating the relative production of linear dipeptides to expression of said candidate polypeptide in said at least one cell or said cell free expression system.


SEQUENCE LISTING (41 sequences)

SEQ ID NO: 1 - protein, 239 aa, Streptomyces noursei
MLAGLVPAPDHGMREEILGDRSRLIRQRGEHALIGISAGNSYFSQKNTVMLLQWAGQRFERTDVVYVDTHIDEMLIADGRSAQEAERSVKRTLKDLRRRLRRSLESVGDHAERFRVRSLSELQETPEYRAVRERTDRAFEEDAEFATACEDMVRAVVMNRPGDGVGISAEHLRAGLNYVLAEAPLFADSPGVFSVPSSVLCYHIDTPITAFLSRRETGFRAAEGQAYVVVRPQELADAA

SEQ ID NO: 2 - protein, 289 aa, Mycobacterium tuberculosis
MSYVAAEPGVLISPTDDLQSPRSAPAAHDENADGITGGTRDDSAPNSRFQLGRRIPEATAQEGFLVRPFTQQCQIIHTEGDHAVIGVSPGNSYFSRQRLRDLGLWGLTNFDRVDFVYTDVHVAESYEALGDSAIEARRKAVKNIRGVRAKITTTVNELDPAGARLCVRPMSEFQSNEAYRELHADLLTRLKDDEDLRAVCQDLVRRFLSTKVGPRQGATATQEQVCMDYICAEAPLFLDTPAILGVPSSLNCYHQSLPLAEMLYARGSGLRASRNQGHAIVTPDGSPAE

SEQ ID NO: 3 - protein, 248 aa, Bacillus subtilis
MTGMVTERRSVHFIAEALTENCREIFERRRHVLVGISPFNSRFSEDYIYRLIGWAKAQFKSVSVLLAGHEAANLLEALGTPRGKAERKVRKEVSRNRRFAERALVAHGGDPKAIHTFSDFIDNKAYQLLRQEVEHAFFEQPHFRHACLDMSREAIIGRARGVSLMMEEVSEDMLNLAVEYVIAELPFFIGAPDILEVEETLLAYHRPWKLGEKISNHEFSICMRPNQGYLIVQEMAQMLSEKRITSEG

SEQ ID NO: 4 - protein, 249 aa, Bacillus licheniformis
MTELIMESKHQLFKTETLTQNCNEILKRRRHVLVGISPFNSRFSEDYIHRLIAWAVREFQSVSVLLAGKEAANLLEALGTPHGKAERKVRKEVSRNRRFAEKALEAHGGNPEDIHTFSDFANQTAYRNLRMEVEAAFFDQTHFRNACLEMSHAAILGRARGTRMDVVEVSADMLELAVEYVIAELPFFIAAPDILGVEETLLAYHRPWKLGEQISRNEFAVKMRPNQGYLMVSEADERVESKSMQEERV

SEQ ID NO: 5 - protein, 239 aa, Bacillus thuringiensis serovar isrealensis
MTNAIAVRNVRKFSSQPLSTNCAEILKRSKHAIIGISPFNSRFSDEYINRLIEWALHTFDDVSVLLAGKEAANLLEALGTPKGKAERKVRKEVSRNRRSAEKALKEHGGNVNAIHTFSDFNDNNAYSCMRAEAEHIFLSETVFRNACLEMSHAAILGRARGTNIDIDQISNDMLNIAVEYVIAELPFFIGGAEILGTQEAVLIYHKPWELGEQIVRNDFSIRMKPNQGYLMVQDMENLS

SEQ ID NO: 6 - protein, 234 aa, Staphylococcus haemolyticus
MQNFKVDFLTKNCKQIYQRKKHVILGISPFTSKYNESYIRKIIQWANSNFDDFSILLAGEESKNLLECLGYSSSKANQKVRKEIKRQIRFCEDEIIKCNKTITNRIHRFSDFKNNIYYIDIYKTIVDQFNTDSNFKNSCLKMSLQALQSKGKNVNTSIEITDETLEYAAQYVLAELPFFLNANPIINTQETLMAYHAPWELGTNIINDQFNLKMNEKQGYIILTEKGDNYVKSV

SEQ ID NO: 7 - protein, 234 aa, Photorhabdus luminescens subsp. laumondii
MLHENSPSFTVQGETSRCDQIIQKGDHALIGISPFNSRFSKDYVVDLIQWSSHYFRQVDILLPCEREASRLLVASGIDNVKAIKKTHREIRRHLRNLDYVISTATLKSKQIRVIQFSDFSLNHDYQSLKTQVENAFNESESFKKSCLDMSFQAIKGRLKGTGQYFGQIDLQLVYKALPYIFAEIPFYLNTPRLLGVKYSTLLYHRPWSIGKGLFNGSYPIQVADKQSYGIVTQL

SEQ ID NO: 8 - protein, 216 aa, Corynebacterium jeikeium
MGESKQEHLIVGVSPFNPRFTPEWLSSAFQWGAERFNTVDVLHPGEISMSLLTSTGTPLGRAKRKVRQQCNRDMRNVEHALEISGIKLGRGKPVLISDYLQTQSYQCRRRSVIAEFQNNQIFQDACRAMSRAACQSRLRVTNVNIEPDIETAVKYIFDELPAYTHCSDLFEYETAALGYPTEWPIGKLIESGLTSLERDPNSSFIVIDFEKELIDD

SEQ ID NO: 9 - protein, 7 aa, artificial sequence
  conserved motif 1
HXXXGXS

SEQ ID NO: 10 - protein, 7 aa, artificial sequence
  conserved motif 2
YXXXEXP

SEQ ID NO: 11 - DNA, 720 bp, Streptomyces noursei
atgcttgcaggcttagttcccgcgccggaccacggaatgcgggaagaaatacttggcgaccgcagccgattgatccggcaacgcggtgagcacgccctcatcggaatcagtgcgggcaacagttatttcagccagaagaacaccgtcatgctgctgcaatgggccgggcagcgtttcgagcgcaccgatgtcgtctatgtcgacacccacatcgacgagatgctgatcgccgacggccgcagcgcgcaggaggccgagcggtcggtcaaacgcacgctcaaggatctgcggcgcagactccggcgctcgctggagagcgtgggcgaccacgccgagcggttccgtgtccggtccctgtccgagctccaggagacccctgagtaccgggccgtacgcgagcgcaccgaccgggccttcgaggaggacgccgaattcgccaccgcctgcgaggacatggtgcgggccgtggtgatgaaccggcccggtgacggcgtcggcatctccgcggaacacctgcgggccggtctgaactacgtgctggccgaggccccgctcttcgcggactcgcccggagtcttctccgtcccctcctcggtgctctgctaccacatcgacaccccgatcacggcgttcctgtcccggcgcgagaccggtttccgggcggccgagggacaggcgtacgtcgtcgtcaggccccaggagctggccgacgcggcctag

SEQ ID NO: 12 - DNA, 870 bp, Mycobacterium tuberculosis
atgtcatacgtggctgccgaaccaggcgtgctgatctcgccgacggacgacttgcagagcccccggtcagccccggcagcgcatgacgaaaatgcggacggcataacaggcgggaccagagacgactctgctcccaactcacggtttcagctaggcaggcgcattccggaagccaccgcccaggaagggtttctggttcggccattcacccaacaatgtcagatcatccacaccgaaggagatcatgctgttatcggggtatccccggggaacagttacttctcccgccagcgcctacgggatctcgggctttggggtctcacgaattttgatcgtgtggacttcgtctacaccgatgtccatgtcgccgagagttacgaagcgctaggcgattccgcaatcgaagcccggcgcaaggcggtcaaaaacatccgcggcgtccgcgccaagatcaccaccacggtgaacgaactcgatccggccggggcccggctgtgcgttcgtccgatgtcggagttccagtccaacgaggcataccgggagctgcatgcggacctgctcacgcgcctgaaagacgacgaggacttgcgcgccgtctgccaggacctagtgcggcgcttcctgtccacgaaagtgggtccgcggcagggggcgacggctactcaagagcaggtgtgcatggactacatttgcgccgaggccccgctattcctcgacacacctgcgattctcggagtgccgtcgtcgttgaattgctaccaccaatcactgcccctcgccgaaatgctctacgcccgaggatcgggactacgggcatcgcgcaatcaaggccacgccattgttacccctgatgggagccccgccgaatga

SEQ ID NO: 13 - DNA, 745 bp, Bacillus subtilis
atgaccggaatggtaacggaaagaaggtctgtgcattttattgctgaggcattaacagaaaactgcagagaaatatttgaacggcgcaggcatgttttggtggggatcagcccatttaacagcaggttttcagaggattatatttacagattaattggatgggcgaaagctcaatttaaaagcgtttcagttttacttgcagggcatgaggcggctaatcttctagaagcgcttggaactccgagaggaaaggctgaacgaaaagtaaggaaagaggtatcacgaaacaggagatttgcagaaagagcccttgtggctcatggcggggatccgaaggcgattcatacattttctgattttatagataacaaagcctaccagctgttgagacaagaagttgaacatgcattttttgagcagcctcattttcgacatgcttgtttggacatgtctcgtgaagcgataatcgggcgtgcgcggggcgtcagtttgatgatggaagaagtcagtgaggatatgctgaatttggctgtggaatatgtcatagctgagctgccgttttttatcggagctccggatattttagaggtggaagagacactccttgcttatcatcgtccgtggaagctgggtgagaagatcagtaaccatgaattttctatttgtatgcggccgaatcaagggtatctcattgtacaggaaatggcgcagatgctttctgagaaacggatcacatctgaaggat

SEQ ID NO: 14 - DNA, 748 bp, Bacillus licheniformis
atgacagagcttataatggagagcaaacaccagctattcaaaaccgaaactcttacccaaaactgcaatgaaatattaaaacgcagacgccatgttctcgtcggcatcagcccgtttaacagccgattttccgaagattatattcatcggcttatcgcctgggccgtccgtgagtttcagagtgtatccgtgcttttggcgggaaaggaagctgccaaccttctcgaagcgctcggcaccccacatgggaaggccgaacggaaagtcaggaaagaagtctcgcggaaccggagattcgctgaaaaggcgttggaagcgcatggcggaaatcccgaggacatccatacattttccgatttcgcgaaccagaccgcataccggaatttgcggatggaagtcgaagctgcctttttcgaccagacgcattttcgcaatgcctgcctggagatgtcgcatgcggctatcctcggacgggcccggggcactcggatggatgtcgtggaagtcagcgcagacatgctggagctggctgttgaatacgtcatcgctgaacttccgtttttcatcgccgcccctgatattttaggcgtcgaagagacgcttcttgcttatcaccggccatggaagctcggcgaacagatctcccgtaatgaatttgccgtcaaaatgcggccgaatcaaggatatctcatggtttccgaagcggacgaaagggtggaatctaaaagcatgcaggaggaacgagtat

SEQ ID NO: 15 - DNA, 720 bp, Bacillus thuringiensis serovar israelensis
atgacgaatgctatagcggtaagaaatgtacgaaagtttagttctcaacccttatctactaattgtgctgaaatattaaaacgtagtaagcatgcaataataggtattagtccgtttaatagtagattttctgatgaatatattaatagactcattgaatgggcattacatacttttgatgatgttagtgttttattagctggaaaagaagctgcaaatttacttgaggctctaggaacaccaaaaggtaaagcggaaagaaaagttaggaaagaagtatctcgaaatagaagatcagctgaaaaggcacttaaagagcatggtggtaatgtaaatgctatccatactttttctgattttaatgacaacaatgcatatagctgcatgagggcagaagcagaacatatttttttaagcgaaactgtttttcgaaatgcttgcttagaaatgtcacatgcagccattttaggtagggcaaggggtactaatatagatattgatcaaatatcaaatgacatgctaaatatcgcagtagaatatgtaattgcagaactcccatttttcattggtggagctgaaattttaggaactcaagaagctgtacttatttatcataaaccatgggagcttggtgaacagatagttagaaatgatttttctatcaggatgaaaccaaatcaaggatatttaatggtacaagacatggaaaatttatcttaa

SEQ ID NO: 16 - DNA, 705 bp, Photorhabdus luminescens subsp. laumondii
atgctgcacgagaattcaccatcatttactgtccaaggtgaaacctctcgttgtgaccaaattattcaaaaaggtgatcacgcgctaatagggataagcccctttaactcgcgtttttcaaaagactatgtagtggaccttattcagtggtcaagtcattatttccgacaagtcgacatattattaccttgtgaacgtgaagcttcacgccttttagtcgctagtggaattgataatgttaaagctatcaaaaaaacacatcgcgaaattagacgtcatttacgtaaccttgattatgttatttccacagcaacattgaaaagtaagcaaatcagagtcatccaatttagtgacttttcactaaaccatgactaccaatctcttaaaacacaagttgaaaacgcgtttaatgaatcagaatcttttaaaaaaagctgtcttgatatgtcctttcaagccataaaagggcgactaaaaggtactgggcaatactttggtcaaattgacctacaattagtatataaagcgttgccatatattttcgctgaaattcctttttacctcaatacccctcgattacttggggtaaagtattctacgttactttatcaccgcccttggtcaatcggaaaagggttatttaacggtagttatcctatacaagtagcagataaacaaagttacggaatcgtcactcaattataa

SEQ ID NO: 17 - DNA, 4139 bp, Artificial
  pQE60-AlbC
ctcgagaaatcataaaaaatttatttgctttgtgagcggataacaattataatagattcaattgtgagcggataacaatttcacacagaattcattaaagaggagaaattaaccatgggacttgcaggcttagttcccgcgccggaccacggaatgcgggaagaaatacttggcgaccgcagccgattgatccggcaacgcggtgagcacgccctcatcggaatcagtgcgggcaacagttatttcagccagaagaacaccgtcatgctgctgcaatgggccgggcagcgtttcgagcgcaccgatgtcgtctatgtcgacacccacatcgacgagatgctgatcgccgacggccgcagcgcgcaggaggccgagcggtcggtcaaacgcacgctcaaggatctgcggcgcagactccggcgctcgctggagagcgtgggcgaccacgccgagcggttccgtgtccggtccctgtccgagctccaggagacccctgagtaccgggccgtacgcgagcgcaccgaccgggccttcgaggaggacgccgaattcgccaccgcctgcgaggacatggtgcgggccgtggtgatgaaccggcccggtgacggcgtcggcatctccgcggaacacctgcgggccggtctgaactacgtgctggccgaggccccgctcttcgcggactcgcccggagtcttctccgtcccctcctcggtgctctgctaccacatcgacaccccgatcacggcgttcctgtcccggcgcgagaccggtttccgggcggccgagggacaggcgtacgtcgtcgtcaggccccaggagctggccgacgcggccagatctcatcaccatcaccatcactaagcttaattagctgagcttggactcctgttgatagatccagtaatgacctcagaactccatctggatttgttcagaacgctcggttgccgccgggcgttttttattggtgagaatccaagctagcttggcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaatttcgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgcatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatttttttaaggcagttattggtgcccttaaacgcctggggtaatgactctctagcttgaggcatcaaataaaacgaaaggctcagtcgaaagactgggcctttcgttttatctgttgtttgtcggtgaacgctctcctgagtaggacaaatccgccgctctagagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcac

SEQ ID NO: 18 - DNA, 4286 bp, Artificial
  pQE60-Rv2275
ctcgagaaatcataaaaaatttatttgctttgtgagcggataacaattataatagattcaattgtgagcggataacaatttcacacagaattcattaaagaggagaaattaaccatggcatacgtggctgccgaaccaggcgtgctgatctcgccgacggacgacttgcagagcccccggtcagccccggcagcgcatgacgaaaatgcggacggcataacaggcgggaccagagacgactctgctcccaactcacggtttcagctaggcaggcgcattccggaagccaccgcccaggaagggtttctggttcggccattcacccaacaatgtcagatcatccacaccgaaggagatcatgctgttatcggggtatccccggggaacagttacttctcccgccagcgcctacgggatctcgggctttggggtctcacgaattttgatcgtgtggacttcgtctacaccgatgtccatgtcgccgagagttacgaagcgctaggcgattccgcaatcgaagcccggcgcaaggcggtcaaaaacatccgcggcgtccgcgccaagatcaccaccacggtgaacgaactcgatccggccggggcccggctgtgcgttcgtccgatgtcggagttccagtccaacgaggcataccgggagctgcatgcggacctgctcacgcgcctgaaagacgacgaggacttgcgcgccgtctgccaggacctagtgcggcgcttcctgtccacgaaagtgggtccgcggcagggggcgacggctactcaagagcaggtgtgcatggactacatttgcgccgaggccccgctattcctcgacacacctgcgattctcggagtgccgtcgtcgttgaattgctaccaccaatcactgcccctcgccgaaatgctctacgcccgaggatcgggactacgggcatcgcgcaatcaaggccacgccattgttacccctgatgggagccccgccgaaagatctcatcaccatcaccatcactaagcttaattagctgagcttggactcctgttgatagatccagtaatgacctcagaactccatctggatttgttcagaacgctcggttgccgccgggcgttttttattggtgagaatccaagctagcttggcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaatttcgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgcatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatttttttaaggcagttattggtgcccttaaacgcctggggtaatgactctctagcttgaggcatcaaataaaacgaaaggctcagtcgaaagactgggcctttcgttttatctgttgtttgtcggtgaacgctctcctgagtaggacaaatccgccgctctagagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcac

SEQ ID NO: 19 - DNA, 4163 bp, Artificial
  pQE60-YvmCsub
ctcgagaaatcataaaaaatttatttgctttgtgagcggataacaattataatagattcaattgtgagcggataacaatttcacacagaattcattaaagaggagaaattaaccatggccggaatggtaacggaaagaaggtctgtgcattttattgctgaggcattaacagaaaactgcagagaaatatttgaacggcgcaggcatgttttggtggggatcagcccatttaacagcaggttttcagaggattatatttacagattaattggatgggcgaaagctcaatttaaaagcgtttcagttttacttgcagggcatgaggcggctaatcttctagaagcgcttggaactccgagaggaaaggctgaacgaaaagtaaggaaagaggtatcacgaaacaggagatttgcagaaagagcccttgtggctcatggcggggatccgaaggcgattcatacattttctgattttatagataacaaagcctaccagctgttgagacaagaagttgaacatgcattttttgagcagcctcattttcgacatgcttgtttggacatgtctcgtgaagcgataatcgggcgtgcgcggggcgtcagtttgatgatggaagaagtcagtgaggatatgctgaatttggctgtggaatatgtcatagctgagctgccgttttttatcggagctccggatattttagaggtggaagagacactccttgcttatcatcgtccgtggaagctgggtgagaagatcagtaaccatgaattttctatttgtatgcggccgaatcaagggtatctcattgtacaggaaatggcgcagatgctttctgagaaacggatcacatctgaaggaagatctcatcaccatcaccatcactaagcttaattagctgagcttggactcctgttgatagatccagtaatgacctcagaactccatctggatttgttcagaacgctcggttgccgccgggcgttttttattggtgagaatccaagctagcttggcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaatttcgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgcatgggcaaatattatacgcaaggcgacaaggtgctgatgccgctggcgattcaggttcatcatgccgtctgtgatggcttccatgtcggcagaatgcttaatgaattacaacagtactgcgatgagtggcagggcggggcgtaatttttttaaggcagttattggtgcccttaaacgcctggggtaatgactctctagcttgaggcatcaaataaaacgaaaggctcagtcgaaagactgggcctttcgttttatctgttgtttgtcggtgaacgctctcctgagtaggacaaatccgccgctctagagctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcac

SEQ ID NO: 20 - DNA, 705 bp, Staphylococcus haemolyticus
gtgcaaaactttaaagtagactttttaacaaaaaactgtaaacaaatctatcaaagaaaaaaacatgtcattttaggaattagcccttttacaagtaagtataacgaatcctacataagaaagattattcaatgggctaattcaaattttgatgatttctctattttattggcaggagaagaatctaaaaatcttttagaatgcttaggatattcatcttctaaagctaatcaaaaagtacgaaaagaaattaaacggcaaatcagattttgtgaagatgaaattataaagtgtaataaaaccataactaatagaattcataggttttctgattttaaaaataatatttattatattgatatatataagactattgtagatcagttcaatacagattctaattttaaaaacagttgtttaaaaatgtcacttcaagcattacaaagcaaaggaaaaaatgttaatacatccatagaaatcactgatgaaactttagagtatgcagcacaatatgttttagcagaattaccattctttttaaatgctaatcccataattaatacacaagaaactttaatggcttatcatgctccatgggaattaggtactaatattataaatgatcagttcaatttaaaaatgaatgaaaaacagggctatattatattaacggaaaaaggggataattatgttaaaagtgtctaa

SEQ ID NO: 21 - DNA, 651 bp, Corynebacterium jeikeium
atgggagaatctaaacaagagcatttgatcgttggcgttagtccgtttaacccacgtttcacaccagagtggctatcttcggcttttcaatggggtgcagaaagatttaacaccgtagatgtacttcatcctggggaaatttccatgtcactgcttacgtcgacaggtacgccactagggagggcaaagagaaaggtgcgtcagcaatgtaatcgtgacatgcgcaacgttgagcatgcccttgaaatatcaggaataaagttaggacgtggcaagccggtactgatttctgactaccttcaaacgcaaagttatcaatgcagacggcgcagtgtgatagctgaatttcagaacaaccagatttttcaggatgcttgtcgtgctatgagtagagctgcatgtcagtcaagactgagggtaacaaacgtgaatatcgagccagatatagaaactgcagtcaaatacatatttgacgagctacccgcctacactcactgcagtgatctctttgaatatgaaacagctgcattgggatatccaaccgaatggccaatagggaagttaatagaatcaggtctgacgtcactggaacgggatccaaatagttcgttcattgttatcgatttcgaaaaggagctaatcgatgattaa

SEQ ID NO: 22 - DNA, 23 bp, Artificial
  Degenerate primer for first conserved motif
cacbysntsntsggsrtswsssc

SEQ ID NO: 23 - DNA, 27 bp, Artificial
  Degenerate primer sequence for second conserved region
atgyasdmscksctcnrsggsmrsawg

SEQ ID NO: 24 - protein, 7 aa, Artificial
  H-[LVA]-[LVI]-[LVI]-G-[VI]-S
HXXXGXS

SEQ ID NO: 25 - protein, 7 aa, Artificial
  Degenerate Protien Motif 2
YXXXEXP

SEQ ID NO: 26 - DNA, 23 bp, Artificial
  Final Degenerate Primer 1
cacbysntsntsggsrtswsssc

SEQ ID NO: 27 - DNA, 27 bp, Artificial
  Final Degenerate Primer 2
gwasrmsggsrnctcskcsmdsaygta

SEQ ID NO: 28 - DNA, 32 bp, Artificial
  AlbC cloning primer 1
agagccatgggacttgcaggcttagttcccgc

SEQ ID NO: 29 - DNA, 29 bp, Artificial
  AlbC cloning primer 2
agagagatctggccgcgtcggccagctcc

SEQ ID NO: 30 - DNA, 32 bp, Artificial
  Rv2275 cloning primer 1
cggccatggcatacgtggctgccgaaccaggc

SEQ ID NO: 31 - DNA, 30 bp, Artificial
  Rv2275 cloning primer 2
ggcagatctttcggcggggctcccatcagg

SEQ ID NO: 32 - DNA, 36 bp, Artificial
  YvmC cloning primer 1
ggcccatggccggaatggtaacggaaagaaggtctg

SEQ ID NO: 33 - DNA, 40 bp, Artificial
  YvmC cloning primer 2
ggcagatcttccttcagatgtgatccgtttctcagaaagc

SEQ ID NO: 34 - protein, 289 aa, Mycobacterium bovis
MSYVAAEPGVLISPTDDLQSPRSAPAAHDENADGITGGTRDDSAPNSRFQLGRRIPEATAQEGFLVRPFTQQCQIIHTEGDHAVIGVSPGNSYFSRQRLRDLGLWGLTNFDRVDFVYTDVHVAESYEALGDSAIEARRKAVKNIRGVRAKITTTVNELDPAGARLCVRPMSEFQSNEAYRELHADLLTRLKDDEDLRAVCQDLVRRFLSTKVGPRQGATATQEQVCMDYICAEAPLFLDTPAILGVPSSLNCYHQSLPLAAMLYARGSGLRASRNQGHAIVTPDGSPAE

SEQ ID NO: 35 - protein, 248 aa, Artificial
  AlbC with c-terminal 6xhis tag and Gly substitution at position 2
MGLAGLVPAPDHGMREEILGDRSRLIRQRGEHALIGISAGNSYFSQKNTVMLLQWAGQRFERTDVVYVDTHIDEMLIADGRSAQEAERSVKRTLKDLRRRLRRSLESVGDHAERFRVRSLSELQETPEYRAVRERTDRAFEEDAEFATACEDMVRAVVMNRPGDGVGISAEHLRAGLNYVLAEAPLFADSPGVFSVPSSVLCYHIDTPITAFLSRRETGFRAAEGQAYVVVRPQELADAARSHHHHHH

SEQ ID NO: 36 - protein, 297 aa, Artificial
  Rv2275 with c-terminal 6xhis tag and substitution of Ala at position 2
MAYVAAEPGVLISPTDDLQSPRSAPAAHDENADGITGGTRDDSAPNSRFQLGRRIPEATAQEGFLVRPFTQQCQIIHTEGDHAVIGVSPGNSYFSRQRLRDLGLWGLTNFDRVDFVYTDVHVAESYEALGDSAIEARRKAVKNIRGVRAKITTTVNELDPAGARLCVRPMSEFQSNEAYRELHADLLTRLKDDEDLRAVCQDLVRRFLSTKVGPRQGATATQEQVCMDYICAEAPLFLDTPAILGVPSSLNCYHQSLPLAEMLYARGSGLRASRNQGHAIVTPDGSPAERSHHHHHH

SEQ ID NO: 37 - protein, 256 aa, Artificial
  YvmC-sub with c-terminal 6his tag and substitution of Ala at position 2
MAGMVTERRSVHFIAEALTENCREIFERRRHVLVGISPFNSRFSEDYIYRLIGWAKAQFKSVSVLLAGHEAANLLEALGTPRGKAERKVRKEVSRNRRFAERALVAHGGDPKAIHTFSDFIDNKAYQLLRQEVEHAFFEQPHFRHACLDMSREAIIGRARGVSLMMEEVSEDMLNLAVEYVIAELPFFIGAPDILEVEETLLAYHRPWKLGEKISNHEFSICMRPNQGYLIVQEMAQMLSEKRITSEGRSHHHHHH

SEQ ID NO: 38 - DNA, 24 bp, Artificial
  AlbC codon for 1st conserved domain
caybynntnntnggnrtnwsnscn

SEQ ID NO: 39 - DNA, 24 bp, Artificial
  AlbC codon for first conserved domain adapted to streptomyces codon usage
cacbysntsntsggsrtswssscs

SEQ ID NO: 40 - DNA, 27 bp, Artificial
  AlbC codons for second conserved domain
tayrtnhkngmngarnysccnkyntwy

SEQ ID NO: 41 - DNA, 27 bp, Artificial
  AlbC codons for second conserved domain using Streptomyces codon usage
tacrtshksgmsgagnysccskystwc